(19) United States
(12) Patent Application Publication
(10) Pub. No.: US 2014/0235548 A1
(43) Pub. Date: Aug. 21, 2014

(54) COMPOSITIONS AND METHODS FOR JAMM PROTEIN INHIBITION

(71) Applicant: Cleave Biosciences, Inc., Burlingame, CA (US)

(72) Inventors: Han-Jie Zhou, Foster City, CA (US); Francesco Parlati, San Francisco, CA (US); Matthieu Roufflet, La Jolla, CA (US); Ethan D. Emberley, Pasadena, CA (US); Raymond J. Deshaies, Pasadena, CA (US); Seth Cohen, San Marcos, CA (US)

(73) Assignee: Cleave Biosciences, Inc., Burlingame, CA (US)

(21) Appl. No.: 14/117,816

(22) PCT Filed: May 10, 2012
(86) PCT No.: PCT/US12/37189

§ 371 Date: May 5, 2014

Related U.S. Application Data
(60) Provisional application No. 61/486,961, filed on May 17, 2011.
(60) Provisional application No. 61/527,487, filed on Aug. 25, 2011.

(30) Foreign Application Priority Data
Apr. 27, 2012 (US) PCT/US2012/035552

(51) Int. Cl. C07K 5/06; C07D 215/36; C07D 215/48; C07D 215/54; C07D 215/50; C07D 215/18; A61K 38/05; C07D 215/42

(52) U.S. Cl. CPC C07K 5/06; A61K 38/05; C07D 215/36; C07D 215/42; C07D 215/54; C07D 215/50; C07D 215/18; C07D 215/48
USPC 514/19.3; 514/311; 514/312; 514/313; 514/314; 546/153; 546/159; 546/169; 546/170; 546/179; 546/180

(57) ABSTRACT

Compounds, pharmaceutical compositions, and methods of using such compounds to treat or prevent diseases or disorders associated with or mediated by JAMM proteins are disclosed. The compounds and compositions inhibit the enzymatic activity of a JAMM domain, including the JAMM domain of the CSN5 subunit of the COP9-signalsome (CSN), the JAMM domain of the Rpn11/Poh1/Psmd14 subunit of the 26S proteasome, the JAMM domain of AMSH, the JAMM domain of AMSH-LP, the JAMM domain of BRCC36, among other JAMM domains.

### CROSS REFERENCE TO RELATED APPLICATIONS

This application claims priority to and benefit of PCT Application Serial Number PCT/US2012/035552 filed Apr. 27, 2012, which claims priority to and benefit of U.S. Provisional Parent Application Ser. Nos. 61/527,487 filed Aug. 23, 2011 and 61/486,961, filed May 17, 2011 the disclosures of which are incorporated herein by reference in their entirety.

### STATEMENT REGARDING FEDERALLY SPONSORED RESEARCH OR DEVELOPMENT

This invention was made with U.S. government support under R01 GM098435 awarded by the National Institutes of Health and an AARA supplement grant number R01 GM065997-0751. The government has certain rights in the invention.

### FIELD OF THE INVENTION

Compounds, pharmaceutical compositions, and methods of using such compounds to treat or prevent diseases or disorders associated with or mediated by JAMM proteins are disclosed. The compounds and compositions inhibit the enzymatic activity of a JAMM domain, including the JAMM domain of the CSN5 subunit of the COP9-signalsome (CSN), the JAMM domain of the Rpn11/Poh1/Psmd14 subunit of the 26S proteasome, the JAMM domain of AMSH, the JAMM domain of AMSH-LP, the JAMM domain of BRCC36, among other JAMM domains. This invention further relates to methods for measuring the activity of a putative inhibitor on CSN, Rpn11, AMSH, AMSH-LP, BRCC36 and other JAMM-containing proteins.

### BACKGROUND

The JAMM domain of the Csn5 subunit of the COP9-signalsome (CSN) comprises a zinc metalloprotease with isopeptidase activity. In addition, to Csn5, JAMM domains are found in Rpn11/Poh1, Brcc36, AMSH, AMSH-LP and several other human proteins. It has been reported that the JAMM domain within the Rpn11 subunit of the 26S proteasome cleaves ubiquitin chains from proteasome substrates as the substrate is being inserted into the 20S proteasome where it is degraded. Mutations in the JAMM domain of Rpn11 may not affect recruitment of substrate to the proteasome, but may block the removal of the ubiquitin chain from the substrate and consequently block degradation of the substrate since it can no longer be inserted into the 20S proteasome due to the bulky ubiquitin chain that remains appended to it.

Nedd8 is an ubiquitin-like (UBL) protein covalently joined to a specific lysine residue of the cullin homology domain subunit of an ubiquitin ligase. Nine different ubiquitin ligases contain a cullin homology domain subunit. These subunits are known as Cu11, Cu12, Cu13, Cu14a, Cu14b, Cu15, Cu17, PARC, and Apc2. With the exception of Apc2, all of these have been reported in the literature to bind and/or be modified by Nedd8. Nedd8 is covalently conjugated to target proteins via as ATPdependent enzyme cascade similar to that used for ubiquitin conjugation. Modification of proteins with Nedd8 is generally thought to be E3-independent, requiring only the Nedd8-specific E1 (Nedd8-activating enzyme; NAE) and E2 (Ubc12/UBE2M or UBE2F). The major cellular “neddylation” substrates are the Cullins, which are scaffolding components of a family of multi-subunit RING finger ligases. Nedd8 modification of Cullin subunits significantly increases the E3 ligase activity of the holoenzyme, both by blocking association of the Cullin with its negative regulator CAND156 and also by directly helping to recruit ubiquitin-charged E2.

Nedd8 that is covalently joined to cullin homology domain proteins can be liberated by the isopeptidase activity specified by the JAMM domain of the Csn5 subunit of the COP9-signalsome (CSN). Compounds targeting components of the ubiquitin-proteasome system may prove useful for the treatment of human malignancies. Specifically, inhibition of the JAMM protein may block the removal of the ubiquitin chain from the substrate and consequently block degradation of the substrate since it can no longer be inserted into the 20S proteasome due to the bulky ubiquitin chain that remains appended to it.

AMSH is responsible for removal of mono-ubiquitin from cell surface tyrosine kinase receptors, such as EGFR. Inhibition of AMSH leads to increased endocytosis of EGFR and downregulation of EGFR activity.

AMSH-LP is responsible for the depolymerization of K63-linked ubiquitin chains. K63-linked ubiquitin chains are added to other cellular proteins thereby altering cellular protein function. For example, proteins that have been modified with K63-linked ubiquin chains are involved in DNA repair, intracellular signaling that includes NFκB signaling and sorting to multivessicular bodies. Inhibition of AMSH-LP activity may alter the balance between K63-ubiqutinated and non-K63-ubiquitated protein.

Inhibition of one of more of these pathways may lead to apoptosis of the cell. Because neoplastic and nondifferentiated cells produce and degrade proteins at a much faster rate than do normal cells, such inhibition may selectively cause apoptosis of neoplastic cells and undifferentiated cells which are also known as cancer cells.

### GOAL OF THE INVENTION

Thus, there is a continuing need for discovering and improving the properties of JAMM protein inhibitors degradation thereby leading to apoptosis of the subject cell. Therefore, it is a goal of the present invention to develop inhibitory compounds that would inhibit on or more of the foregoing pathways of protein

### SUMMARY OF THE INVENTION

These and other objects and needs are achieved by the aspects, embodiments and features of the present invention including compounds, pharmaceutical compositions, methods of treatment, methods of cellular analysis and methods of diagnosis according to the present invention.

Provided herein are aspects and embodiments of the invention including compounds that will inhibit the enzymatic activity of a JAMM domain (hereinafter compounds of the invention or compounds), including the JAMM domain of the Csn5 subunit of the COP9-signalsome (CSN), the JAMM domain of the Rpn11/Poh1/Psmd14 subunit of the 26S proteasome, the JAMM domain of AMSH, the JAMM domain of AMSH-LP, the JAMM domain BRCC36, or any other JAMM domain.

In a first embodiment of the invention, these compounds are organic molecules having a pharmacophore moiety comprised of the fragment X—C—C—Y wherein the X group is nitrogen and the Y group is oxygen or sulfur. The carbon atoms of the pharmacophore moiety are saturated or unsaturated or a mixture thereof. The organic molecule has an aromatic, aliphatic or aliaromatic ring framework with the X group being in the ring and the Y group of the C—Y moiety being a substituent appended to the ring, or the organic molecule has an aromatic, aliphatic or aliaromatic ring framework with both of X and Y being substituents appended to the framework. The aromatic, aliphatic or aliaromatic framework is a single 5 or 6 membered ring, or a 5:5, 5:6, 6:5 or 6:6 member aromatic, aliphatic or aliaromatic bicyclic ring. The rings can include heteroatoms other than X and Y. The count of ring members includes the carbons and heteroatoms in the ring.

The rings for the organic molecule framework may be aliphatic including saturated, or unsaturated, or saturated in part and unsaturated in part, or may be aromatic, or may be saturated and/or unsaturated in part (i.e., aliphatic) and aromatic in part. A ring framework that is aliphatic in part and aromatic in part is termed herein “aliaromatic.” An example is 2,3-dihydrobenzofuran. In these situations, the X and Y substituents are appropriately bonded.

Each individual ring framework described above and each individual ring structure drawn below may be selected as an individual, independent, separate aspect of this first embodiment of the invention without inclusion of any other ring framework or drawn structure of this first embodiment. Any combination of the ring frameworks and drawn ring structures may also be selected without inclusion of any non-selected framework or drawn structure. The choice of the character of the individual independent ring frameworks extends to the independent choice of aromatic, aliphatic or aliaromatic character alone or to any combination of the aliphatic, aromatic or aliaromatic characterization as well. The aromatic and aliaromatic characterizations are preferred. For example, the first embodiment may be a single 5 membered ring alone as described above. Alternatively, the first embodiment may be a single six membered ring alone as described above. Or, the first embodiment can be any one of the 5:5, 5:6, 6:5 or 6:6 member bicyclic rings alone as described above. Any combination of these ring frameworks may also be selected. Preferred ring frameworks include the aliphatic, aromatic and aliaromatic bicyclic ring frameworks, especially the aliphatic, aromatic and aliaromatic 5:6 and 6:6 member frameworks, more especially the aliphatic, aromatic and aliaromatic 6:6 member frameworks, and most especially an aromatic 6:6 member framework.

The selection of X and Y may be independently made as single choices, or as any combination in any order from the list of atoms identified for X and Y.

In particular, the frameworks of the first embodiment of the invention can be exemplified as any one or more of the individual ring structures drawn below. These individually drawn ring structures more particularly illustrate but do not limit the ring frameworks. Any individual, drawn ring structure may be selected independently and alone as a framework of the first embodiment of the invention, or any one or more of the drawn structures in any order and in any combination may be selected. For the ring structures drawn below, the reversal of the positions of X and Y as substituents on a ring is also included but not shown. Insertion of X at the fused ring junction is also included but not shown.

When the rings are all carbon, some of those rings may be within the group of rings set forth in the Definitions section for the terms “Cycloalkyl” and “Aryl” or “Aromatic.” In addition, the “cycloalkyl” ring may be unsaturated with one or two olefinic groups when it is an organic molecule ring framework.

When the rings are all carbon and include X as nitrogen within the rings, the rings in part may be within the group of rings set forth in the Definitions section for the terms “Heterocycle” and “Heteroaryl.” Additional heteroatoms may be present in the rings according to ordinary and appropriate chemical principles. The additional heteroatoms may include nitrogen, oxygen and/or sulfur. One, two, three or four heteroatoms may be present and may all be the same kind of atom or may be a combination of the foregoing atoms. The resulting rings include but are not limited to the “Heteroaryl” and “Heterocycle” groups set forth in the Definitions Section.

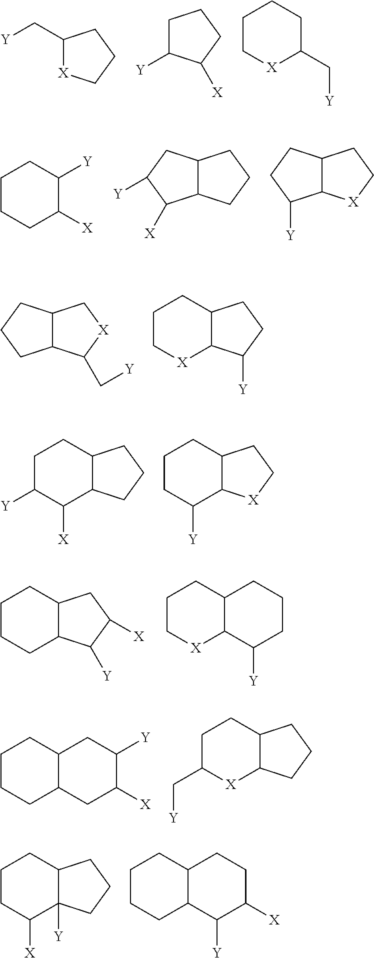

In the first embodiment, the compounds are also characterized by the following provisions. When X as nitrogen is in the aromatic, aliphatic or aliaromatic framework, it is either a secondary or tertiary nitrogen. When X as nitrogen is a substituent appended to the aromatic, aliphatic or aliaromatic framework, it is a primary, secondary or tertiary nitrogen. When Y is oxygen or sulfur and is a substituent appended to the aromatic, aliphatic or aliaromatic framework, it is either an hydroxyl, thiol, ether or thioether group or the ether oxygen or ether sulfur of an ester or thioester respectively, or it is the carbonyl oxygen or thiocarbonyl sulfur or an amide or thioamide group respectively. Additionally, when Y is mercaptan, the compound for administration is the dimer formed through coupling of two mecaptan groups together to form the disulfide.

In this first embodiment, the aromatic, aliphatic or aliaromatic framework and the drawn rings may be optionally substituted by one or more chemical substituents chosen from the list of aliphatic, aromatic and functional substituents given in the following DEFINITIONS section. Preferably the number of chemical substituents is one to four, more preferably one to two and most preferably one. Each individual, separate ring framework and each individual, separate drawn ring structure can be substituted by any individual independent selection of any substituent or substituents. In other words, while these substituents are provided herein as lists, selection of one or more individual substituents from such lists may be made independently for each individual, separate ring framework or drawn ring structure. For example, a 5:6 aromatic ring with nitrogen in the ring may be substituted by halogen alone, may be substituted by carboxyl alone, may be substituted by aryl carboxyl alone, or may be substituted by amine and carboxyl.

Selections from the DEFINITIONS section are to be made according to known chemical principles and functions. These selections include but are not limited to the following substituents to be chosen individually, independently and alone, or in any order of two or more of the individual substituents or in any combination thereof. The substituents include but are not limited to halogen, CN, optionally substituted carboxyl, optionally substituted ester, optionally substituted amine, optionally substituted amide, optionally substituted thioamide, optionally substituted aliphatic or aryl carboxyl, optionally substituted aliphatic or aryl carboxyamide, optionally substituted aminoalkylamine, carboxyl, ester, alkyl aliphatic ester, amine, optionally substituted aliphatic diamine, optionally substituted aminoalkyl carboxyl, optionally substituted amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs, optionally substituted alkyl, optionally substituted alkenyl, optionally substituted alkynyl, optionally substituted alkoxy, optionally substituted alkthioxy, optionally substituted alkoxyalkyl; optionally substituted alkoxaryl; optionally substituted amino, optionally substituted carbonyl, optionally substituted carboxylic acid, optionally substituted alkoxyheteroaryl, optionally substituted alkoxheterocycyl, optionally substituted aryl, optionally substituted heteroaryl, optionally substituted heterocyclyl, optionally substituted alkaryl, optionally substituted alkyheteroaryl, optionally substituted alkheterocyclyl, optionally substituted carbocyclyl, optionally substituted alkylcarbocyclyl, and any combination thereof. Included as example of such substituents are carboxyl, ester, alkylcarboxyl, alkyl alkenyl ester, amine, alkylenyl diamine, aminoalkyl carboxyl, amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs and these examples optionally substituted by functional groups as given in the DEFINITIONS section. These examples are not meant to exclude other substituents that are set forth in the DEFINITIONS section as available for substitution on the ring framework of the organic compounds of this first embodiment.

The compounds of this first embodiment include as well, the form of pharmaceutically acceptable salts, the N-oxide derivatives, protected derivatives, individual isomers, mixture of isomers thereof and mixtures of any of the foregoing with pharmaceutically acceptable solvents as applicable according to the chemistry of the organic molecule involved. The salts, derivatives, isomers and mixtures may be independently, individually selected alone or in any combination or order thereof.

However, in this first embodiment, the organic molecule is not aminothiophene, hydroxylthiophene, thiolthiophene, thiol or hydroxyl pyridine, thiolpyrrole, hydroxylpyrrole, 2-aminomethylthiophene, 2-hydroxymethyllthiophene, 2-thio or hydroxymethyl-pyridine, thio or hydroxyl or aminomethyl-pyrimidine, thio or hydroxyl or aminomethyl-triazine, 2-thio or hydroxymethyl-pyrrole, thio or hydroxyl or aminomethyl-imidazole, thio or hydroxyl or aminomethyl-oxazole, thio or hydroxyl or aminomethyl-thiazole, thio or hydroxyl or aminomethyl-isoxazole, thio or hydroxyl or aminomethyl-isothiazole, a C₁ to C₆ alkyl or alkoxy substituted aforementioned derivatives, halo substituted substituted aforementioned derivatives, a phenyl or phenoxy substituted substituted aforementioned derivatives, an hydroxyl or thiol substituted substituted aforementioned derivatives, a C₁ to C₃ carboxylic acid or carboxyl ester substituted aforementioned derivatives, 8-quinolinethiol, a C₁ to C₆ alkyl or alkoxy substituted 8-quinolinethiol, a halo substituted 8-quinolinethiol, a phenyl or phenoxy substituted 8-quinolinethiol, an amino substituted 8-quinolinethiol, an hydroxyl or thiol substituted 8-quinolinethiol, a C₁ to C₃ carboxylic acid or carboxyl ester substituted 8-quinolinethiol, or the disulfide dimers thereof.

In a second embodiment, the compounds are organic molecules having a pharmacophore moiety, S—C—C—N wherein the organic molecule is 8-quinolinethiol, or a derivative thereof, or a dimer of 8-quinolinethiol or of a derivative of 8-quinolinethiol dimer, each with one or more peptide components appended to the quinolinethiol framework. These organic molecules may be each individually and independently selected as the second embodiment such that the second embodiment may independently be any one of these organic molecules. The optional peptide components include one or more peptide substituents at the 2, 3, 4, 5, 6 and/or 7 positions of the 8-quinolinethiol framework. The peptide substituent may be an optionally substituted peptidyl group containing from 1 to 6 natural and/or non-natural amino acid moieties. The peptidyl group may be directly bonded to the framework, or may be bonded to the 8-quinolinethiol framework through a linker. The linker may be an alkyl amide group or an alkyl ester group wherein the amide or ester moiety forms the linking bond to the peptide substituent and the alkyl moiety is directly bonded to the 8-quinolinethiol framework or is bonded to the 8-quinolinethiol framework through an amide group, an ester group, an ether group or an amine group. The peptide substituent or substituents are capable of interacting with the JAMM metalloprotease domain or a biological complex containing the JAMM metalloprotease domain. This peptide substituent or optionally substituted peptide substituent differentiates this embodiment from the compounds of the first embodiment and negates the exclusory proviso given in connection with the first embodiment.

In this second embodiment, the derivative is constructed as the 8-quinolinethiol framework or the dimer thereof with one or more appended chemical substituents chosen from the substituents given in the DEFINITIONS section according to standard chemical principles. Preferably, the 8-quinolinethiol framework may be optionally substituted by one or more chemical substituents chosen from the following list of selections.

Selections from the DEFINITIONS section are to be made according to known chemical principles and functions. These selections include but are not limited to the following substituents to be chosen individually, independently and alone, or in any order of two or more of the substituents or in any combination thereof. The substituents include but are not limited to halogen, CN, optionally substituted carboxyl, optionally substituted amine, optionally substituted amide, optionally substituted thioamide, optionally substituted aliphatic or aryl carboxyl, optionally substituted aliphatic or aryl carboxyamide, optionally substituted aliphatic or aryl ester, optionally substituted aminoalkylamine, optionally substituted ester, alkyl aliphatic ester, optionally substituted aliphatic diamine, optionally substituted aminoalkyl carboxyl, optionally substituted amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs, optionally substituted alkyl, optionally substituted alkenyl, optionally substituted alkynyl, optionally substituted alkoxy, optionally substituted alkthioxy, optionally substituted alkoxalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkylheteroaryl, optionally substituted alkheterocyclyl, optionally substituted, carbocyclyl, optionally substituted alkylcarbocyclyl, and any combination thereof. Included as examples of such, substituents are carboxyl, ester, alkylcarboxyl, alkyl alkenyl ester, amine, alkylenyl diamine, aminoalkyl carboxyl, amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs and these examples optionally substituted by functional groups as given in the DEFINITIONS section. These examples are not meant to exclude other substituents that are set forth in the DEFINITIONS section as available for substitution on the ring framework of the organic compounds of this second embodiment.

The compounds of this second embodiment include as well, the compounds in the form of their pharmaceutically acceptable salts, the N-oxide derivatives, protected derivatives, individual isomers, mixture of isomers thereof and mixtures of any of the foregoing with pharmaceutically acceptable solvents as applicable according so the chemistry of the organic molecule involved. The salts, derivatives, isomers and mixtures may be independently, individually selected alone or in any combination or order thereof.

In a third embodiment, the compounds are organic molecules which are derivatives of 3-hydroxyl 4H-pyran-4-thione or 3-hydroxypyridine-4(1H)-thione. The derivative comprises the 3-hydroxyl-4H-pyran-4-thione or 3-hydroxylpyridine-4(1H)-thione framework with one or more appended chemical substituents. The compounds of this third embodiment may be selected individually, independently and alone without inclusion of other compounds of the third embodiment or may be chosen in any combination or in any order thereof.

The chemical substituents appended to the framework of the third embodiment include one or more appended chemical substituents chosen from the substituents given in the Definitions section according to standard chemical principles. Preferably, the framework of this third embodiment may be optionally substituted by one or more chemical substituents chosen from the following list of selections. Preferably, the number of substituents is 1 through 4, more preferably 1 or 2, most preferably 1.

Selections from the DEFINITIONS section are to be made according to known chemical principles and functions. These selections include but are not limited the following substituents to be chosen individually, independently and alone, or in any order of two or more of the substituents or in any combination thereof. The substituents include but ate not limited to halogen, CN, optionally substituted carboxyl, optionally substituted amine, optionally substituted amide, optionally substituted thioamide, optionally substituted aliphatic or aryl carboxyl, optionally substituted aliphatic or aryl carboxyamide, optionally substituted aliphatic or aryl ester, optionally substituted aminoalkylamine, optionally substituted ester, alkyl aliphatic ester, optionally substituted aliphatic diamine, optionally substituted aminoalkyl carboxyl, optionally substituted amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs, optionally substituted alkyl, optionally substituted alkenyl, optionally substituted alkynyl, optionally substituted alkoxy, optionally substituted alkthioxy, optionally substituted alkoxyalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkylheteroaryl, optionally substituted alkheterocyclyl, optionally substituted carbocyclyl, optionally substituted alkylcarbocyclyl, and any combination thereof. Included as examples of such substituents are carboxyl, ester, alkylcarboxyl, alkyl alkenyl ester, amine, alkylenyl diamine, aminoalkyl carboxyl, amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs and these examples optionally substituted by functional groups as given in the DEFINITIONS section. These examples are not meant to exclude other substituents that are set forth in the DEFINITIONS section as available for substitution on the ring framework of the organic compounds of this third embodiment.

However, for this third embodiment, the organic molecule is not 3-hydroxyl-4-pyrothione, 3-hydroxyl-2-carboxyl-4-pyrothione, 3-hydroxyl-2-carboxamindo-4-pyrothione or 3-hydroxypyridine-4-(1H)-thione.

The compounds of this third embodiment include as well, the inhibitory compounds in the form of their pharmaceutically acceptable salts, the N-oxide derivatives, protected derivatives, individual isomers, mixture of isomers thereof and mixtures of any of the foregoing with pharmaceutically acceptable solvents as applicable according to the chemistry of the organic molecule involved. The salts, derivatives, isomers and mixtures may be independently, individually selected, alone or in any combination or order thereof.

In a fourth embodiment, the compounds are organic molecules having the formulas 3-hydroxyl-4H-pyran-4-thione or 3-hydroxypyridine-4(1H)-thione or a derivative thereof and the derivative comprises the 3-hydroxyl-4H-pyran-4-thione or 3-hydroxypyridine-4(1H)-thione framework with one or more appended chemical substituents. The compounds of this fourth embodiment may be selected individually, independently and alone without inclusion of other compounds of the fourth embodiment or may be chosen in any combination or in any order thereof.

The chemical substituents for this fourth framework include one or more appended chemical substituents chosen from the substituents given in the Definitions section according to standard chemical principles. Preferably, the framework may be optionally substituted by one or more chemical substitutents chosen from the following list of selections. Preferably the number of substituents is 1 through 4, more preferably 1 or 2, most preferably 1.

Selections from the DEFINITIONS section are to be made according to known chemical principles and functions. These selections include but are not limited to the following substituents to be chosen individually, independently and alone, or in any order of one or more of the substituents or in any combination thereof. The substituents include but are not limited to halogen, CN, optionally substituted carboxyl, optionally substituted amine, optionally substituted amide, optionally substituted thioamide, optionally substituted aliphatic or aryl carboxyl, optionally substituted aliphatic or aryl carboxyamide, optionally substituted aliphatic or aryl ester, optionally substituted aminoalkylamine, optionally substituted ester, alkyl aliphatic ester, optionally substituted aliphatic diamine, optionally substituted aminoalkyl carboxyl, optionally substituted amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs, optionally substituted alkyl, optionally substituted alkenyl, optionally substituted alkynyl, optionally substituted alkoxy, optionally substituted alkthioxy, optionally substituted alkoxalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkyheteroaryl, optionally substituted alkheterocyclyl, optionally substituted carbocyclyl, optionally substituted alkylcarbocyclyl, and any combination thereof. Included as examples of such substituents are carboxyl, ester, alkylcarboxyl, alkyl alkenyl ester, amine, alkylenyl diamine, aminoalkyl carboxyl, amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs and these examples optionally substituted by functional groups as given in the DEFINITIONS section. These examples are not meant to exclude other substituents that are set forth in the DEFINITIONS section as available for substitution on the aromatic or aliphatic framework of the organic compounds of this fourth embodiment.

In this fourth embodiment, the 3-hydroxyl-4H-pyran-4-thione or 3-hydroxypyridine-4(1H)-thione or derivative thereof further comprises one or more peptide substituents at the 2, 4, 5 and/or 6 positions of the 3-hydroxyl-4H-pyran-4-thione or 3-hydroxypyridine-4(1H)-thione framework. The peptide substituent is an optionally substituted peptidyl group containing from 1 to 6 natural and/or non-natural amino acid moieties. The peptidyl group may be directly bonded to the 3-hydroxyl-4H-pyran-4-thione or 3-hydroxypyridine-4(1H)-thione framework or bonded through a linker. The linker may be an alkyl amide group or an alkyl ester group wherein the amide or ester moiety forms the linking bond to the peptide substituent and the alkyl moiety is directly bonded to the 3-hydroxyl-4-pyrothione framework or is bonded to the 3-hydroxyl-4-pyrothione framework through an amide group, an ester-group, an ether group or an amine group. The peptide substituent or substituents may be capable of interacting with the JAMM metalloprotease domain or a biological complex containing the JAMM metalloprotease domain. This peptide substituent or optionally substituted peptide substituent differentiates this embodiment from the compounds of the third embodiment.

The compounds of this fourth embodiment include as well, the compounds in the form of their pharmaceutically acceptable salts, the N-oxide derivatives, protected derivatives, individual isomers, mixture of isomers thereof and mixtures of any of the foregoing with pharmaceutically acceptable solvents as applicable according to the chemistry of the organic molecule involved. The salts, derivatives, isomers and mixtures may be independently, individually selected alone or in any combination or order thereof.

In a fifth embodiment, the compounds includes catechol ketone of the formula:

(RO)₂C₆H₃—CO—CZ═CHY or (RO)₂C₆H₃—CO—CHZC(═O)Y

wherein R is H or C₁ to C₆ alkyl, Z is cyano, nitro, halo or trifluoromethyl and Y is phenyl, substituted phenyl, aminophenyl, 1-indolyl catecholyl, pyridyl, naphthyl or quinolinyl. The compounds of this fifth embodiment may be selected individually, independently and alone without inclusion of other compounds of the fifth embodiment or may be chosen in any combination or in any order thereof.

The compounds of this fifth embodiment include as well, the compounds in the form of their pharmaceutically acceptable salts, the N-oxide derivatives, protected derivatives, individual isomers, mixture of isomers thereof and mixtures of any of the foregoing with pharmaceutically acceptable solvents, as applicable according to the chemistry of the organic molecule involved. The salts, derivatives, isomers and mixtures may be independently, individually selected alone or in any combination or order thereof.

In the foregoing embodiments 1-5 of the invention, the compounds can be combined with a zinc cation to form a metal chelate with a K_{eq} less than 1 millimolar, as shown by a UV-visible absorption analysis of a solution of the organic molecule alone and the organic molecule completed with zinc cation in buffered aqueous medium. The UV-visible absorption analysis is conducted to determine absorption maxima for the organic molecule alone as λᵢ and the organic molecule in a saturated chelate with zinc cation as and the equilibrium constant K_{eq} being determine by monitoring the absorption at λᵢ and λ_{f} while titrating zinc cation into an aqueous solution of the organic molecule and calculating the equilibrium constant according to the equation K_{∞} equals the concentration of the organic molecule-Zn chelate divided by the multiple of the concentrations of the organic molecule alone and the free Zn cation.

In all or the foregoing embodiments of the invention, the compounds display an inhibition activity against a JAMM metalloprotease domain. In particular, the above described organic molecules exhibit at least about a 50% inhibition of metalloprotease activity of a JAMM metalloprotease domain containing protein alone or as part of a signalosome complex or part of a 26S proteasome complex, which inhibition is determined by conducting a biochemical assay of the ability of the compound to inhibit the ability of the JAMM metalloprotease domain to cleave a monoubiquitin, a multiubiquitin chain or a ubiquitin-like modifier from a protein substrate or ubiquitin from a K63-linked ubiquitin chain, the concentration of the organic molecule being no more than about 500 micromolar.

Another embodiment of the invention includes a method for screening for a compound that inhibits the metalloprotease activity of a protein containing a JAMM metalloprotease domain. The method includes the steps of selecting a candidate from the organic molecules described above including all variations and individual embodiments, aspects, examples and characterizations thereof to provide a selected candidate, and testing the selected candidate in a JAMM domain inhibition assay. The inhibition assay includes the following steps.

The first step calls for combining (a) a JAMM enzymatic material selected from the group consisting of a JAMM domain containing protein, a signalosome complex and a 26S proteasome complex containing the JAMM protein, and (b) a protein substrate selected from the group consisting of a protein modified by a ubiquitin, a protein modified by a ubiquitin-like modifier and a protein modified by a ubiquitin chain to produce an enzymatic medium, wherein the protein substrate is modified with a tag that is detectable by measurement of molecular weight, spectroscopic interaction or chromatographic R_{f} determination.

The second step calls for conducting a first measurement of the enzymatic medium relative to the protein substrate alone wherein the first measurement is made by a detection of the tag.

The third step calls for combining the selected candidate with the protein substrate and adding the JAMM enzymatic material to produce a candidate medium.

The fourth step calls for conducting a second measurement of the candidate medium relative to the protein substrate alone wherein the second measurement is made by detection of the tag.

The fifth step calls for comparing the first and second measurements to identify a candidate that demonstrates at least about a 50% inhibition at a concentration of no more than 500 micromolar in the candidate medium, the difference between the first and second measurements being at least about 50% with the second measurement being greater than the first measurement.

A further embodiment of the invention includes an assay for determining the inhibition of JAMM metalloprotease domain activity by a potential inhibitory candidate. The assay includes the following steps.

The first step calls for combining a JAMM enzymatic material selected from the group consisting of a JAMM domain containing protein, a signalosome complex and a 26S proteasome complex containing the JAMM protein, and a protein substrate selected from the group consisting of a protein modified by a ubiquitin, a protein modified by a ubiquitin-like modifier and a protein modified by a ubiquitin chain to produce an enzymatic medium wherein the protein substrate is modified with a tag that is detectable by measurement of molecular weight, spectroscopic interaction or chromatographic R_{f} determination.

The second step calls for conducting a first measurement of the enzymatic medium relative to the protein substrate alone wherein the first measurement is made by a detection of the tag.

The third step calls for combining a potential inhibitory candidate with the protein substrate and adding the JAMM enzymatic material to produce a candidate medium.

The fourth step calls for conducting a second measurement of the candidate medium relative to the protein substrate alone wherein the second measurement is made by detection of the tag.

The fifth step calls for comparing the first and second measurements to identify a candidate that demonstrates at least about a 50% inhibition at a concentration of no more than 500 micromolar in the candidate medium, the difference between the first and second measurements being at least about 50% with the second measurement being greater than the first measurement.

Another embodiment of the invention includes a method of diagnosing responsiveness to administration of an inhibitor of enzymatic activity of a JAMM domain. The method includes the steps of (i) obtaining cells from a patient with a disease that is potentially responsive to inhibition of the JAMM domain, (ii) exposing said cells to an inhibitor of enzymatic activity of a JAMM domain, and (iii) comparing the response of the cells to the response by cells from a patient with a positive response to administration of the inhibitor of enzymatic activity of a JAMM domain.

A method of using the aforementioned compounds for treating a disorder characterized by an inappropriate level of CRL or proteasome activity or AMSH activity or AMSH-LP activity or BRCC-36 activity or any other JAMM-containing enzymatic activity or in which a reduction of the normal level of CRL or proteasome or AMSH or AMSH-LP or BRCC-36 or any other JAMM-containing enzyme activity yields a clinical benefit. This disorder can include cancer or immune disorders characterized by excessive cell proliferation or cellular signaling. This method includes treatment of human cancers that overexpress c-Myc, express an oncogenic form of the K-Ras protein. This method also includes treatment of cancers that overexpress Csn5, or in cancers sustained by mutations in genes that encode proteins that function upstream of K-Ras, including but not limited to those driven by mutations in BRAF or EGFR.

### BRIEF DESCRIPTION OF THE DRAWINGS

FIG. 1 depicts inhibition of Rpn11 activity in cells by compound 1 (‘test compound’).

FIG. 2 depicts inhibition of CSN5 activity in vitro by Compound 1 (“test compound”) and corresponding IC50 curve.

FIG. 3 depicts accumulation of neddylated forms of Cullins 1, 2, and 4A in HEK-293T cells treated with compound 1 (‘test compound 1’) and compound 101 ‘(test compound 2’).

FIG. 4 depicts the Rpn11-reaction schematic, progress curve and dose response curves with select compounds, including compound 1.

FIG. 5 depicts structure-activity relationships for compound 1: evaluation of the chelation activity of compound 1 on Rpn11's biochemical activity.

FIGS. 6A and B depict a list of compound IDs, structures and corresponding IC₅₀ values in biochemical Rpn11 and Csn5 assays and a cell-based tumor cell growth assay.

FIG. 7 depicts IC₅₀ values as determined for compound 1 in the Rpn11 Assay.

FIG. 8 depicts an orthogonal method for monitoring Rpn11 reactions. Rpn11 reaction substrate and products were separated by SDS-PAGE electrophoresis and detected by immunoblot of ubiquitin (upper panel) or fluorescence detection of Oregon Green (lower panel).

FIG. 9 depicts the Csn5 Assay.

FIG. 10 depicts progress curves for reactions containing enzyme (Csn5), substrate (SCF^{Skp2}-Nedd8OG) and either DMSO (green) or inhibitor (red). In this example, inhibitor refers to compound 001 (100 μM).

FIG. 11 depicts the IC₅₀ determination for compound 001 on Csn5.

FIG. 12 depicts an orthogonal method for monitoring Csn5 reaction.

FIG. 13 depicts a cell-based Csn5 Assay. Treatment of cells with compound 1 causes an accumulation of Cu11-Nedd8 and depletion of Cu11.

FIG. 14 depicts an AMSH-LP assay. Compound 1 causes inhibition of conversion of di-ubiqutin K63 to mono-ubiquitin by AMSH-LP.

### DETAILED DESCRIPTION OF THE INVENTION

The novel features of the invention are set forth with particularity in the appended claims. A better understanding of the features and advantages of the present invention will be obtained by reference to the following detailed description that sets forth illustrative embodiments, in which the principles of the invention are utilized. The accompanying drawings further illustrate the invention.

Definitions

Unless defined otherwise, all technical and scientific terms used herein have the same meaning as commonly understood by a person of ordinary skill in the art.

As used in the specification and the appended claims, the singular terms “a,” “an” and “the” include plural referents unless the context clearly dictates otherwise.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

As used herein, “individual” (as in the subject of the treatment) or “patient” means both mammals and non-mammals. Mammals include, for example, humans; non-human primates, e.g. apes and monkeys; and non-primates, e.g. dogs, cats, cattle, horses, sheep, and goats. Non-mammals include, for example, fish and birds.

The term “may” in the context of this application means “is permitted to” or “is able to” and is a synonym for the term “can.” The term “may” as used herein does not mean possibility or chance.

The term “disease” or “disorder” or “malcondition” are used interchangeably, and are used to refer to diseases or conditions wherein X plays a role in the biochemical mechanisms involved in the disease or malcondition or symptom(s) thereof such that a therapeutically beneficial effect can be achieved by acting on X. “Acting on” X, or “modulating” X, can include binding to X and/or inhibiting the bioactivity of X and/or allosterically regulating the bioactivity of X in vivo.

The expression “effective amount”, when used to describe therapy to an individual suffering from a disorder, refers to the amount of a drug, pharmaceutical agent or compound of the invention that will elicit the biological or medical response of a cell, tissue, system, animal or human that is being sought, for instance, by a researcher or clinician. Such responses include but are not limited to amelioration, inhibition or other action on a disorder, malcondition, disease, infection or other issue with or in the individual's tissues wherein the disorder, malcondition, disease and the like is active, wherein such inhibition or other action occurs to an extent sufficient to produce a beneficial therapeutic effect. Furthermore, the term “therapeutically effective amount” means any amount which, as compared to a corresponding subject who has not received such amount, results in improved treatment healing, prevention, or amelioration of a disease, disorder, or side effect, or a decrease in the rate of advancement of a disease or disorder. The term also includes within its scope amounts effective to enhance normal physiological function.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

Phrases such as “under conditions suitable to provide” or “under conditions sufficient to yield” or the like, in the context of methods of synthesis, as used herein refers to reaction conditions, such as time, temperature, solvent, reactant concentrations, and the like, that are within ordinary skill for an experimenter to vary, that provide a useful quantity or yield of a reaction product. It is not necessary that the desired reaction product be the only reaction product or that the starting materials be entirely consumed, provided the desired reaction product can be isolated or otherwise further used.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

An “analog” of a chemical structure, as the term is used herein, refers to a chemical structure that preserves substantial similarity with the parent structure, although it may not be readily derived synthetically from the parent structure. A related chemical structure that is readily derived synthetically from a parent chemical structure is referred to as a “derivative.”

When a substituent is specified to be an atom or atoms of specified identity, “or a bond”, a configuration is referred to when the substituent is “a bond” that the groups that are immediately adjacent to the specified substituent are directly connected to each other in a chemically feasible bonding configuration.

All chiral, diastereomeric, racemic forms of a structure are intended, unless a particular stereochemistry or isomeric form is specifically indicated. In several instances though an individual stereoisomer is described among specifically claimed compounds, the stereochemical designation does not imply that alternate isomeric forms are less preferred, undesired, or not claimed. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

As used herein, the terms “stable compound” and “stable structure” are meant to indicate a compound that is sufficiently robust to survive isolation to a useful degree of purity from a reaction mixture, and formulation into an efficacious therapeutic agent. Only stable compounds are contemplated herein.

Selected substituents within the compounds described herein are present to a recursive degree. In this context “recursive substituent” means that a substituent may recite another instance of itself. Because of the recursive nature of such substituents, theoretically, a large number may be present in any given claim. One of ordinary skill in the art of medicinal chemistry and organic chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis. Recursive substituents are an intended aspect of the disclosed subject matter. One of ordinary skill in the art of medicinal and organic chemistry understands the versatility of such substituents. To the degree that recursive substituents are present in a claim of the disclosed subject matter, the total number should be determined as set forth above.

When a group is recited, wherein the group can be present in more than a single orientation within a structure resulting in more than single molecular structure, e.g., a carboxamide group C(═O)NR, it is understood that the group can be present in any possible orientation, e.g., X—C(═O)N(R)—Y or X—N(R)C(═O)—Y, unless the context clearly limits the orientation of the group within the molecular structure.

When a group, e.g., an “alkyl” group, is referred to without any limitation on the number of atoms in the group, it is understood that the claim is definite and limited with respect the size of the alkyl group, both by definition; i.e., the size (the number of carbon atoms) possessed by a group such as an alkyl group is a finite number, less than the total number of carbon atoms in the universe and bounded by the understanding of the person of ordinary skill as to the size of the group as being reasonable for a molecular entity; and by functionality, i.e., the size of the group such as the alkyl group is bounded by the functional properties the group bestows on a molecule containing the group such as solubility in aqueous or organic liquid media. Therefore, a claim reciting an “alkyl” or other chemical group or moiety is definite and bounded, as the number of atoms in the group cannot be infinite.

In general, “substituted” refers to an organic group as defined herein in which one or more bonds to a hydrogen atom contained therein are replaced by one or more bonds to a non-hydrogen atom. More particularly, the term “chemical substituent” refers to any and all aliphatic, aromatic and functional groups listed in this section that can be appended to an organic molecule. A functional group is an inorganic moiety such as halogen, sulfate, nitro, amino and the like as well as monocarbon functional groups such as carboxyl, carbonyl, carboxamide that are ordinary and typical optional substituents of organic molecules. In the context of this invention, recitation of this term without indication of specific groups constitutes the definition given above. Recitation of this term in combination with a Markush recitation of specific groups constitutes a subgenus of the understanding conveyed by the foregoing definition. The term “substituent” generally means any appropriate group named below that has a “yl”, “y” or “o” ending to designate that it is appended, attached or covalently bonded to another moiety such as but not limited to an aromatic framework. Examples include but are not limited to, a halogen (i.e., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents J that can be bonded to a substituted carbon (or other) atom include F, Cl, Br, I, OR′, OC(O)N(R′)₂, CN, NO, NO₂, ONO₂, azido, CF₃, OCF₃, R′, O (oxo), S (thiono), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen, or a carbon-based moiety, and wherein the carbon-based-moiety can itself be further substituted; for example, wherein R′ can be hydrogen, alkyl acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or R′ can be independently mono- or multi-substituted with J; or wherein two R′ groups bonded to a nitrogen atom or to adjacent nitrogen atoms can together with the nitrogen atom or atoms form a heterocyclyl, which can be mono- or independently multi-substituted with J.

In various embodiments, J can be halo, nitro, cyano, OR, NR₂, or R, or is C(O)OR, C(O)NR₂, OC(O)OR, OC(O)NR₂, N(R)C(O)OR, N(R)C(O)NR₂ or thio/thiono analogs thereof. By “thio/thiono analogs thereof”, with respect to a group containing an O, is meant that any or all O atoms in the group can be replaced by an S atom; e.g., for group C(O)OR, a “thio/thiono analog thereof” includes C(S)OR, C(O)SR, and C(S)SR; e.g., for group OC(O)NR₂, a “thio/thiono analog thereof” includes SC(O)NR₂, OC(S)NR₂, and SC(S)NR₂; and so forth.

When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, which can also be written as “CO”, “C(O)”, or “C(═O)”, wherein the C and the O aw double bonded. When a carbon atom is substituted with a double-bonded oxygen (═O) group, the oxygen substituent is termed an “oxo” group. When a divalent substituent such as NR is double-bonded to a carbon atom, the resulting C(═NR) group is termed an “imino” group. When a divalent substituent such as S is double-bonded to a carbon atom, the results C(═S) group is termed a “thiocarbonyl” or “thiono” group.

Alternatively, a divalent substituent such as O or S can be connected by two single bonds to two different carbon atoms. For example, O, a divalent substituent, can be bonded to each of two adjacent carbon atoms to provide an epoxide group, or the O can form a bridging ether group, termed an “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR′₂)ₙ wherein n is 1, 2, 3, or more, and each R′ is independently selected.

The term “Aliphatic” refers to any organic group that is non-aromatic. Included are acyclic and cyclic organic compounds composed of carbon, hydrogen and optionally of oxygen, nitrogen, sulfur and other heteroatoms. This term encompasses all of tire following organic groups except the following defined aromatic and heteroaromatic groups. Examples of such groups include but are not limited to alkyl, alkenyl, alkynyl, corresponding groups with heteroatoms, cyclic analogs, heterocyclic analogs, branched and linear versions and such groups optionally substituted with functional groups, as these groups and others meeting this definition of “aliphatic” are defined below.

The term “Aromatic” refers to any and all aromatic groups including but not limited to aryl, aralkyl, heteroalkylaryl, heteroalkylheteroaryl and heteroaryl groups. The term “aromatic” is general in that it encompasses all compounds containing aryl groups (all carbon aromatic groups) and all compounds containing heteroaryl groups (carbon-heteroatom aromatic groups), as these groups and others meeting this definition of “aromatic” are defined below.

As used herein, the term “optionally” means that the corresponding substituent or thing may or may not be present. It includes both possibilities.

“Alkyl” refers to a straight or branched hydrocarbon chain radical consisting solely of carbon and hydrogen atoms, containing no unsaturation, having from one to ten carbon atoms (e.g., C₁-C₁₀ alkyl). Whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated. In some embodiments, it is a C₁-C₄ alkyl group. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl, decyl, and the like. The alkyl is attached to the rest of the molecule by a single bond, for example, methyl (Me), ethyl (Et), n-propyl, 1-methylethyl(iso-propyl), n-butyl, n-pentyl, 1,1-dimethylethyl(t-butyl), 3-methylhexyl, 2-methylhexyl, and the like.

Unless stated otherwise specifically in the specification, an alkyl group is optionally substituted by one or more of substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, OR^{a}, —SR^{a}, —OC(O)—R^{a}, —N(R^{a})₂, —C(O)R^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} ) where t is 1 or 2), —S(O)ₜN(R^{a})₂ (where t is 1 or 2), or PO₃(R^{a})₂ where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl)heterocycyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e. C₂-C₁₀ alkenyl). Whenever it appears herein, a numerical range such as “2 to 10” refers to each integer in the given range; e.g., “2 to 10 carbon atoms” means that the alkenyl group may consist of 2 carbon atoms, 3 carbon atoms, etc., up to and including 10 carbon atoms. In certain embodiments, an alkenyl comprises two to eight carbon atoms. In other embodiments, an alkenyl comprises two to five carbon atoms (e.g., C₂-C₅ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

Unless stated otherwise specifically in the specification, an alkenyl group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, OR^{a}, —SR^{a}, —OC(O)—R^{a}, —N(R^{a})₂, —C(O)R^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Alkenyl-cycloalkyl” refers to an -(alkenyl)cycloalkyl radical where alkenyl and cyclo alkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e. C₂-C₁₀ alkynyl). Whenever it appears herein, a numerical, range such as “2 to 10” refers to each integer in the given range; e.g., “2 to 10 carbon atoms” means that the alkynyl group may consist of 2 carbon atoms, 3 carbon atoms, etc., up to and including 10 carbon atoms. In certain embodiments, an alkynyl comprises two to eight carbon atoms. In other embodiments, an alkynyl has two to five carbon atoms (e.g., C₂-C₅ alkynyl). The alkynyl is attached to the rest of the molecule by a single bond, for example, ethynyl, propynyl, butynyl, pentynyl, hexynyl, and the like.

Unless stated otherwise specifically in the specification, an alkynyl group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, OR^{a}, —SR^{a}, —OC(O)—R^{a}, —N(R^{a})₂, —C(O)R^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl,

“Alkynyl-cycloalkyl” refers to refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. C₂-C₁₀ cycloalkyl). Whenever it appears herein, a numerical range such as “3 to 10” refers to each integer in the given range; e.g., “3 to 10 carbon atoms” means that the cycloalkyl group may consist of 3 carbon atoms, etc., up to and including 10 carbon atoms. In some embodiments, it is a C₃-C₈ cycloalkyl radical. In some embodiments, it is a C₃-C₅ cycloalkyl radical. Illustrative examples of cycloalkyl groups include, but are not limited to the following moieties: cyclopropyl, cyclobutyl, cyclopentyl, cyclopentenyl, cyclohexyl, cyclohexenyl, cycloseptyl, cyclooctyl, cyclononyl, cyclodecyl, norbornyl, and the like.

Unless stated otherwise specifically in the specification, a cycloalkyl group is optionally substituted by one or more substituents as defined above. Such substituents further independently include: alkyl, heteroalkyl, alkenyl, alkynyl, cycloalkyl, heterocycloalkyl, aryl, arylalkyl heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Cycloalkyl-alkenyl” refers to a -(cycloalkyl)alkenyl radical where cycloalkyl and heterocycloalkyl are as disclosed herein and which are optionally substituted by one or more of the substituents described as suitable substituents for heterocycloalkyl and cycloalkyl respectively.

“Cycloalkyl-heterocycloalkyl” refers to a -(cycloalkyl)heterocycyl radical where cycloalkyl and heterocycloalkyl are as disclosed herein and which are optionally substituted by one or more of the substituents described as suitable substituents for heterocycloalkyl and cycloalkyl respectively.

“Cycloalkyl-heteroaryl” refers to a -(cycloalkyl) heteroaryl radical where cycloalkyl and heterocycloalkyl are as disclosed herein and which are optionally substituted by one or more of the substituents described as suitable substituents for heterocycloalkyl and cycloalkyl respectively.

The term “alkoxy” refers to the group —O-alkyl, including from 1 to 8 carbon atoms of a straight, branched, cyclic configuration, and combinations thereof attached to the parent structure through an oxygen. Examples include methoxy, ethoxy, propoxy, isopropoxy, cyclopropyloxy, cyclohexyloxy and the like. “Lower alkoxy” refers to alkoxy groups containing one to six carbons. In some embodiments, C₁-C₄ alkyl is an alkyl group which encompasses both straight and branched chain alkyls of from 1 to 4 carbon atoms.

The term “substituted alkoxy” refers to alkoxy wherein the alkyl constituent is substituted (i.e., —O-(substituted alkyl)).

Unless stated otherwise specifically in the specification, the alkyl moiety of an alkoxy group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), or PO₃(R^{a})₂ where each R^{a} is independently hydrogen, alkyl, fluoroalkyl, carbocyclyl, carbocyclalkyl, aryl, aralkyl, heterocycloalkyl, heterocyclylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a C₁-C₆ alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group. In some embodiments, C₁-C₄ alkoxy, is an alkoxy group which encompasses both straight and branched chain alkoxy groups of from 1 to 4 carbon atoms.

The term “substituted alkoxycarbonyl” refers to the group (substituted alkyl)-O—C(O)— wherein the group is attached to the parent structure through the carbonyl functionality.

Unless stated otherwise specifically in the specification, the alkyl moiety of an alkoxycarbonyl group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)—, and (heterocycloalkyl)-C(O)—, wherein the group is attached to the parent structure through the carbonyl functionality. In some embodiments, it is a C₁-C₁₀ acyl radical which refers to the total number of chain or ring atoms of the alkyl, aryl, heteroaryl or heterocycloalkyl portion of the acyloxy group plus the carbonyl carbon of acyl, i.e., three other ring or chain atoms plus carbonyl. If the R radical is heteroaryl or heterocycloalkyl, the hetero ring or chain atoms contribute to the total number of chain or ring atoms.

Unless stated otherwise specifically in the specification, the “R” of an acyloxy group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Acyloxy” refers to a R(C═O)O— radical wherein “R” is alkyl, aryl, heteroaryl, heteroalkyl, or heterocycloalkyl, which are as described herein. In some embodiments, it is a C₁-C₄ acyloxy radical which refers to the total number of chain or ring atoms of the alkyl, aryl, heteroaryl or heterocycloalkyl portion of the acyloxy group plus the carbonyl carbon of acyl, i.e. three other ring or chain atoms plus carbonyl. If the R radical is heteroaryl or heterocycloalkyl, the hetero ring or chain atoms contribute to the total number of chain or ring atoms.

Unless stated otherwise specifically in the specification, the “R” of an acyloxy group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2 —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl, unless stated otherwise specifically in the specification. When a —N(R^{a})₂ group has two Ra other than hydrogen they can be combined with the nitrogen atom to form a 4-, 5-, 6-, or 7-membered ring. For example, —N(R^{a})₂ is meant to include, but not be limited to, 1-pyrrolidinyl and 4-morpholinyl.

Unless stated otherwise specifically in the specification, an amino group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl and each of these moieties may be optionally substituted as defined herein.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid. The person skilled in the art is familiar with reaction conditions for carrying out the N-oxidation.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions, and amines, within the meaning herein.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. In some embodiments it is a C₁-C₄ amido or amide radical, which includes the amide carbonyl in the total number of carbons in the radical. The R₂ of —N(R)₂ of the amide may optionally be taken together with the nitrogen to which it is attached to form a 4-, 5-, 6-, or 7-membered ring. Unless stated, otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound of Formula (1), thereby forming a prodrug. Any amine, hydroxy, or carboxyl side chain on the compounds described herein can be amidified. The procedures and specific groups to make such amides are known to those of skill in the art and can readily be found in reference sources such as Greene and Wuts, Protective Groups in Organic Synthesis, 3rd Ed., John Wiley & Sons, New York, N.Y., 1999, which is incorporated herein by reference in its entirety.

“Aryl” refers to a conjugated pi radical with six or ten ring atoms which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. The term includes monocyclic or fused-ring polycyclic (i.e., rings which share adjacent pairs of ring atoms) groups.

Unless stated otherwise specifically in the specification, an aryl moiety is optionally substituted by one or snore substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl cycloalkyl, aryl, heteroaryl (bonded through a ring carbon) and heteroalicyclic (bonded through a ring carbon). Any amine, hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3rd Ed., John Wiley & Sons, New York, N.Y., 1999, which is incorporated herein by reference in its entirety.

Unless state otherwise specifically in the specification, an ester group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given, e.g. C₁-C₄ heteroalkyl which refers to the chain length in total, which in this example is 4 atoms long. For example, a —CH₂OCH₂CH₃ radical is referred to as a “C₄” heteroalkyl, which includes the heteroatom center in the atom chain length description. Connection to the rest of the molecule may be through either a heteroatom or a carbon in the heteroalkyl chain.

A heteroalkyl group may be substituted with one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

“Heteroalkylaryl” refers to as -(heteroalkyl)aryl radical where heteroalkyl and aryl are as disclosed herein and which are optionally substituted by one or more of the substituents described as suitable substituents for heteroalkyl and aryl respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl respectively.

“Heteroalkylheterocycloalkyl” refers to an -(heteroalkyl)heterocycloalkyl radical where heteroalkyl and heteroaryl are as disclosed herein and which are optionally substituted by one or more of the substituents described as suitable substituents for heteroalkyl and heterocycloalkyl respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl respectively.

“Heteroaryl” refers to a 5, 6 or 10-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range refers to each integer in the given range. An N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to adeninyl, azabenzimidazolyl, azaindolyl, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl),benzothieno[3,2-d]pyrimidinyl, benzotriazolyl, benzo[4,6]imidazo[1,2-a]pyridinyl, carbazolyl, cinnolinyl, cyclpenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, imidazopyridinyl, isoxazolopyridinyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thianaphthalenyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., theinyl), xanthinyl, guaninyl, quinoxalinyl, and quinazolinyl groups.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiopenyl (2-thienyl, 3-thienyl), furyl (2-furyl, 3-furyl), indolyl, oxadiazolyl, isoxazolyl, quinazolinyl, fluorenyl, xanthenyl, isoindanyl, benzyhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl) 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl(2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl(2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl), 3-benzo[b]thiophenyl), 4-benzo[b]thiophenyl), 5-benzo[b]thiophenyl), 6-benzo[b]thiophenyl), 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl, indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepine-1-yl, 5H-dibenz[b,f]azepine-2-yl, 5H-dibenz[b,f]azepine-3-yl, 5H-dibenz[b,f]azepine-4yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Unless stated otherwise specifically in the specification a heteraryl moiety is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl.

Substituted heteroaryl also includes ring systems substituted with one or more oxide (—O—)substituents, such as pyridinyl N-oxides.

“Heterocyclyl” refers to any monocyclic or polycyclic moiety comprising at least one heteroatom selected from nitrogen, oxygen and sulfur. As used herein, heterocyclyl moieties can be aromatic or nonaromatic.

Unless stated otherwise, heterocyclyl moieties are optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heteroaryl or heteroarylalkyl.

“Heteroarylalkyl” refers to a moiety having an aryl moiety, as described herein, connected to an alkylene moiety, as described herein, wherein the connection to the remainder of the molecule is through the alkylene group.

“Heterocyclylalkyl” refers to a stable 5, 6 or 10-membered non-aromatic ring radical having from one to six heteroatoms selected from nitrogen, oxygen and sulfur. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiaxolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

Unless stated otherwise specifically in the specification, a heterocycloalkyl moiety is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heteroaryl or heteroarylalkyl.

“Heterocyclylalkyl” also includes bicyclic ring systems wherein one non-aromatic ring, usually with 3 to 7 ring atoms, contains at least 2 carbon atoms in addition 1-3 heteroatoms independently selected from oxygen, sulfur, and nitrogen, as well as combinations comprising at least one of the foregoing heteroatoms; and the other ring, usually with 3 so 7 ring atoms, optionally contains 1-3 heteroatoms independently selected from oxygen, sulfur, and nitrogen and is not aromatic.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is —CF₃.

The term “(Cₓ-C_{y})perfluoroalkylene,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkylene, more preferred is —(C₁-C₃)perfluoroalkylene, most preferred is —CF₂—.

“Sulfanyl” refers to the groups: —S-(optionally substituted alkyl), —S-(optionally substituted aryl), —S-(optionally substituted heteroaryl), and —S-(optionally substituted heterocycloalkyl).

“Sulfinyl” refers to the groups: —S(O)H, —S(O)-(optionally substituted alkyl), —S(O)-(optionally substituted amino), —S(O)-(optionally substituted aryl), —S(O)-(optionally substituted heteroaryl), and —S(O)-(optionally substituted heterocycloalkyl).

“Sulfonyl” refers to the groups: —SO(O₂)—H, —S(O₂)-(optionally substituted alkyl), —S(O₂)-optionally substituted amino), —S(O₂)-optionally substituted aryl), —S(O₂)-optionally substituted heteroaryl), and —S(O₂)-optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6-, or 7-membered ring. In some embodiments, it is a C₁-C₁₀ sulfonamido, wherein each R is sulfonamido contains 1 carbon, 2 carbons, 3 carbons, or 4 carbons total. A sulfonamido group is optionally substituted by one or more of the substituents described for alkyl, cycloalkyl, aryl, heteroaryl respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl respectively.

The term “azido” refers to an N₃ group. An “azide” can be an organic azide or can be a salt of the azide (N₃⁻) anion. The term “nitro” refers to an NO₂ group bonded to an organic moiety. The term. “nitroso” refers to an NO group bonded to an organic moiety. The term nitrate refers to an ONO₂ group bonded to an organic moiety or to a salt of the nitrate (NO₃⁻) anion.

The term “urethane” (“carbamoyl” or “carbamyl”) includes N— and O— urethane groups, i.e. —NRC(O)OR and —OC(O)NR₂ groups, respectively.

The term “sulfonamide” (or “sulfonamido”) includes S— and N— sulfonamide groups, i.e., —SO₂NR₂ and —NRSO₂R groups, respectively. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

The term “amidine” or “amidino” includes groups of the formula —C(NR)NR₂. Typically, an amidino group is —C(NH)NH₂.

The term “guanidine” or “guanidino” includes groups of the formula —NRC(NR)NR₂. Typically, a guanidino group is —NHC(NH)NH₂.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt termed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic,stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula (I) compounds, for example in their purification by recrystallization. All of these salts may be prepared by conventional means from the corresponding compound according to Formula (I) by reacting, for example, the appropriate acid or base with the compound according to Formula (I). The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

A “hydrate” is a compound that exists in a composition with water molecules. The composition can include water in stoichiometric quantities, such as a monohydrate or a dihydrate, or can include water in random amounts. As the term is used herein a “hydrate” refers to a solid form, i.e., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometric or non-stoichiometric. As the term is used herein a “solvate” refers to a solid form, i.e., a compound in solution in a solvent, while it may be solvated, is not a solvate as the term is used herein.

A “prodrug” as is well known in the art is a substance that can be administered to a patient where the substance is converted in vivo by the action of biochemicals within the patients body, such as enzymes, to the active pharmaceutical ingredient. Examples of prodrugs include esters of carboxylic acid groups, which can be hydrolyzed by endogenous esterases as are found in the bloodstream of humans and other mammals. Conventional procedures for the selection and preparation of suitable prodrug derivatives are described, for example, in “Design of Prodrugs”, ed. H. Bundgaard, Elsevier, 1985.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected, from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

The term “amino protecting group” or “N-protected” as used herein refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

The term “hydroxyl protecting group” or “O-protected” as used hereto refers to those groups intended to protect an OH group against undesirable reactions during synthetic procedures and which can later be removed to reveal the amine. Commonly used hydroxyl protecting groups are disclosed in Protective Groups in Organic Synthesis, Greene, T. W.; Wuts, P. G. M. John Wiley & Sons, New York, N.Y., (3rd Edition, 1999). Hydroxyl protecting groups include acyl groups such as formyl, acetyl, propionyl, pivaloyl, t-butylacetyl, 2-chloroaacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. It is well within the skill of the ordinary artisan to select and use the appropriate hydroxyl protecting group for the synthetic task at hand.

Standard abbreviations for chemical groups such as well known in the art are used: e.g., Me=methyl, Et=ethyl, i-Pr=isopropyl, Bu=butyl, t-Bu=tert-butyl, Ph=phenyl, Bn=benzyl, Ac=acetyl, Bz=benzoyl, and the like.

### COMPOUNDS

The present invention is directed in part to compounds to inhibit the enzymatic activity of a JAMM domain (hereinafter inhibitory compounds), including the JAMM domain of the CSN5 subunit of the COP9-signalsome (CSN), the JAMM domain of the Rpn11/Poh1/Psmd14 subunit of the 26S proteasome, the JAMM domain of AMSH, the JAMM domain of AMSH-LP, the JAMM domain of BRCC36, or any other JAMM domain.

These compounds are suitable for inhibiting the activity of a JAMM metalloprotease domain (hereinafter inhibitory compounds). They include any of the following organic molecules disclosed as aspects of the invention. The embodiments of the invention disclosed In the Summary of the Invention are subgroups of these aspects of the invention.

As a first aspect of the invention, there are disclosed inhibitory compounds suitable for inhibiting the activity of a JAMM metalloprotease domain which are composed of organic molecules having a zinc chelating pharmacophore moiety of the fragment X—C—C—Y or X—C—Y. A preferred subgroup of this first aspect is the first embodiment described in the Summary of the Invention.

For this first aspect of the invention, the following designations apply.
The X group is nitrogen, oxygen or sulfur.
The Y group is nitrogen, oxygen or sulfur.

The carbon atom or atoms of the pharmacophore moiety are saturated or unsaturated or a mixture thereof.

The organic molecule has an aromatic, aliphatic or aliaromatic ring framework with one of the X and Y groups being part of the ring framework and the other being a substituent appended to the ring framework, or both of X and Y groups being substituents appended to the ring framework, or both of the X and Y groups being part of the ring framework.

The aromatic, aliphatic or aliaromatic framework is a single 5 or 6 member aromatic or aliphatic ring or a 5:5, 5:6, 6:5 or a 6:6 member aromatic, aliphatic or aliaromatic bicyclic ring, the count of members including the carbons and heteroatoms in the ring. The aromatic, aliphatic or aliaromatic framework may be independently chosen from any two or more of these rings in any order, in any combination, or may be chosen, alone as each single independent framework without inclusion of any other ring framework. The selection of X and Y may be independently made as single choices, or as any combination in any order from the list of atoms identified for X and Y. The selection of aliphatic, aromatic or aliaromatic may be chosen as individual, independent characterizations alone without inclusion of the other characterizations or may be chosen as two or more characterizations in any order and in any combination.

The aromatic, aliphatic or aliaromatic ring framework may also be optionally substituted by any one or more of the chemically appropriate substituents as defined above in the “DEFINITIONS” section and as set forth for the first embodiment of the Summary of the Invention. Preferably with this option, 1 to 4 chemical substituents may be present, more preferably 1 or 2, most preferably 1. The substituents may be each independently chosen as single substituents or may be chosen in any order or any combination from the foregoing respective sections defining such substituents.

The compounds of this first embodiment are characterized by the following provisions.
1. When X is sulfur or oxygen and is a substituent appended to the ring framework, it is either a thiol, hydroxyl, thioether or ether group or a double bonded sulfur or oxygen of a thiocarbonyl or carbonyl group respectively.
2. When X is sulfur or oxygen and is in the ring framework, it may be bonded to two carbons as in thiophene or furan or to carbon and nitrogen, or to carbon and oxygen (sulfur only) or to carbon and sulfur.
3. When X and/or Y is nitrogen and is in the ring framework, it is either a secondary or tertiary nitrogen.
4. When X and/or Y is nitrogen and is a substituent appended to the ring framework, it is a primary, secondary or tertiary nitrogen.
5. When Y as oxygen or sulfur is in the ring framework, it is covalently bonded to two carbons, or to carbon and nitrogen, or to carbon and sulfur.
6. When Y is oxygen or sulfur and is a substituent appended to the ring framework, it is either an hydroxyl, thiol, ether or thioether group or the oxygen or sulfur of a carbonyl or thiocarbonyl group respectively.

The group of ring structures encompassing this pharmacophore according to the foregoing description includes the following structures shown below. Each individual ring structure drawn below may be chosen as individual, separate independent ring for a compound of this first embodiment of the invention or multiple drawn ring structures may be chosen in any order and in any combination. The same selection, choice and designation guidelines for the ring frameworks apply equally to the drawn ring structures of these frameworks. Reversal of the positions of X and Y as substituents on a ring is also included but not shown. Insertion of X or Y at the fused ring junction is also included but not shown. The rings for the organic molecule framework may be aliphatic including saturated, unsaturated, or saturated and unsaturated in part or may be aromatic or may be saturated and/or unsaturated in part and aromatic in part (aliaromatic). In these situations, the X and Y substituents are appropriately bonded.

When the rings are all carbon, some of these rings may be within the group of rings set forth in the Definitions section for the term “Cycloalkyl.” In addition, the “cycloalkyl” ring may be unsaturated with one or two olefinic groups when it is an organic molecule ring framework.

When the rings are all carbon and include X as nitrogen within the rings, the rings in part may be within the group of rings set forth in the Definitions section for the term “Heterocycle” Additional heteroatoms may be present in the rings according to ordinary and appropriate chemical principles. The additional heteroatoms may include nitrogen, oxygen and/or sulfur. One, two, three or four heteroatoms may be present and may all be the same kind of atom or may be a combination of the foregoing atoms. The resulting rings include but are not limited to the “Heteroaryl” and “Heterocycle” groups set forth in the Definitions Section.

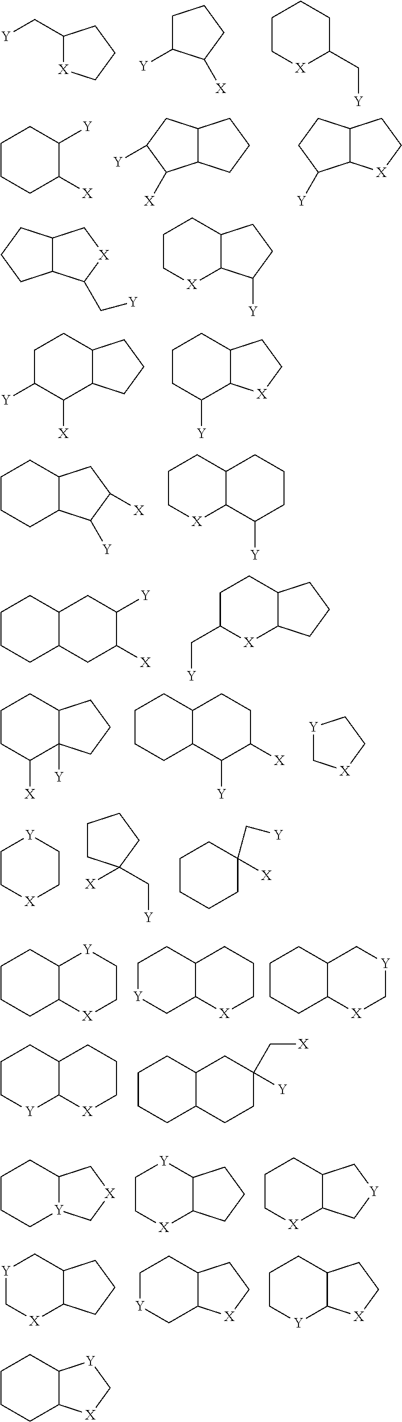

However, in this first aspect the organic molecule is not aminothiophene, hydroxylthiophene, thiolthiophene, thiol or hydroxyl pyridine, thiolpyrrole, hydroxylpyrrole, 2-aminomethylthiophene, 2-hydroxymethyllthiophene, 2-thio or hydroxymethyl-pyridine, thio or hydroxyl or aminomethyl-pyrimidine, thio or hydroxyl or aminomethyl-triazine, 2-thio or hydroxymethyl-pyrrole, thio or hydroxyl or aminomethyl-imidazole, thio or hydroxyl or aminomethyl-oxazole, thio or hydroxyl or aminomethyl-thiazole, thio or hydroxyl or aminomethyl-isoxazole, thio or hydroxyl or aminomethyl-isothiazole, a C₁ to C₆ alkyl or alkoxy substituted aforementioned derivatives, halo substituted substituted aforementioned derivatives, a phenyl or phenoxy substituted substituted aforementioned derivatives, an hydroxyl or thiol substituted substituted aforementioned derivatives, a C₁ to C₃ carboxylic acid or carboxyl ester substituted aforementioned derivatives, 8-quinolinethiol, a C₁ to C₆ alkyl or alkoxy substituted 8-quinolinethiol, a halo substituted 8-quinolinethiol, a phenyl or phenoxy substituted 8-quinolinethiol, an amino substituted 8-quinolinethiol, an hydroxyl or thiol substituted 8-quinolinethiol, a C₁ to C₃ carboxylic acid or carboxyl ester substituted 8-quinolinethiol, or the disulfide dimers thereof.

In this first aspect, these compounds having the foregoing chemical substituent or substituents do not cause a significant decrease in the inhibitory activity relative to the inhibitory activity of a standard compound, 8-quinolinethiol, in the JAMM domain inhibition assay.

In a second aspect of the invention, there are disclosed inhibitory compounds suitable for inhibiting the activity of a JAMM metalloprotease domain. These compounds include an organic molecule having a zinc chelating pharmacophore moiety formed of the fragment X—C—C—Y as defined above in the first aspect of the invention. The organic molecule for this second aspect is a 6:6 bicyclic aromatic, aliphatic or aliaromatic ring framework with one or more peptide substituents appended to the framework. The aromatic, aliphatic or aliaromatic characterization of the framework may be individually and independently chosen alone without inclusion of the other characterizations or may be any two or more characterizations in any order and any combination. The ring framework may also optionally be substituted by chemical substituents as described above for the first embodiment of the invention including the selection of one or more substituents independently and individually chosen from the substituents for the first embodiment. Preferably with this option, 1 to 4 chemical substituents are present, more preferably 1 or 2, most preferably 1.

A preferred subgroup of this second aspect is second embodiment disclosed to the Summary of the Invention, namely the 8-quinoline thiol or dimer thereof or derivatives thereof, each with one or more peptide substituents.

In this second aspect, the 6:6 bicyclic ring framework with one or more peptide substituents has the one or more peptide substituents bonded to the 2, 3, 4, 5, 6 and/or 7 positions of the 6:6 bicyclic framework. The peptide substituent is an optionally substituted peptidyl group containing from 1 to 6 natural and/or non-natural amino acid moieties. The peptidyl group is bonded to the 8-quinolinethiol framework through a linker. The linker is an alkyl amide group or an alkyl ester group wherein the amide or ester moiety forms the linking bond to the peptide substituent and the alkyl moiety is directly bonded to the 8-quinolinethiol framework or is bonded to the 8-quinolinethiol framework through an amide group, an ester group, an ether group or an amine group. The peptide substituent or substituents is capable of interacting with the JAMM metalloprotease domain or a biological complex containing the JAMM metalloprotease domain. Preferably one, two or three peptide substituents are present in this second aspect, more preferably one or two.

In this second aspect, these compounds having the peptide substituent and the optional chemical substituent or substituents do not cause a significant decrease in the inhibitory activity relative to the inhibitory activity of a standard compound, 8-quinolinethiol, in the JAMM domain inhibition assay.

The third aspect of the invention includes the derivatives of 4H-pyran-4-thione or 3-hydroxypyridine-4(1H)-thione as disclosed for the third embodiment of the invention set forth in the Summary of the Invention individually, independently and singly selected as well as selected in any combination.

The fourth aspect of the invention includes the peptide substituted pyran or pyiridine compounds of the fourth embodiment of the invention disclosed in the Summary of the Invention individually, independently and singly selected as well as selected in any combination. These compounds are the 3-hydroxyl-pyran or pyridine or derivative thereof of with one or more peptide substituents at the 2, 4, 5 and/or 6 positions of the 3-hydroxyl-4H-pyran-4-thione or 3-hydroxypyridine-4(1H)-thione framework as disclosed in the Summary of the Invention.

The fifth aspect of the invention includes the catechol ketones of the fifth embodiment of the invention disclosed in the Summary of the Invention, individually, independently and singly selected as well as selected in any combination. Included are the catechol ketones themselves as well as those catechol ketones further bearing one of more peptide substituents as disclosed in the Summary of the Invention.

In the foregoing aspects 1-5, the disclosed organic molecules in combination with a zinc cation form a metal chelate with a K_{eq} less than 1 millimolar, as shown by a UV-visible absorption analysis of a solution of the organic molecule alone and the organic molecule completed with zinc cation in buffered aqueous medium, the UV-visible absorption analysts being conducted to determine absorption maxima for the organic molecule alone as λᵢ and the organic molecule in a saturated chelate with zinc cation as and the equilibrium constant K_{eq} being determine by monitoring the absorption at λᵢ and λ_{f} while titrating zinc cation into an aqueous solution of tire organic molecule and calculating the equilibrium constant according to the equation K_{eq} equals the concentration of the organic molecule-Zn chelate divided by the multiple of the concentrations of the organic molecule alone and the free Zn cation.

In all of the foregoing aspects, the disclosed organic molecules also exhibit at feast about a 50% inhibition of metalloprotease activity of a JAMM metalloprotease domain containing protein alone or as part of a signalosome complex or part of a 26S proteasome complex, which inhibition is determined by conducting a biochemical assay of the ability of the compound to inhibit the ability of the JAMM metalloprotease domain to cleave a monoubiquitin, a multiubiquitin chain or a ubiquitin-like modifier from a protein substrate or ubiquitin from a K63-linked ubiquitin chain, the concentration of the organic molecule being no more than about 500 micromolar.

For all of the foregoing aspects wherein the disclosed compound includes a peptide substituent, the peptide substituent may be an epitopal substrate for the JAMM metalloprotease domain or may be a single chain hypervariable region chain from a humanized or chimeric monoclonal antibody to the protein containing the JAMM metalloprotease domain or the signalsome complex containing protein.

### Further Preferred Chemical Substituents

A) In all of the foregoing aspects wherein the organic molecule is appended with one or more chemical substituents, the chemical substituent(s) may be any as described with each aspect. Preferably, the chemical substituent(s) are individually and independently selected from the group consisting of halogen, optionally substituted alkyl, optionally substituted alkoxy, optionally substituted alkthioxy, optionally substituted amino, optionally substituted alkyl diamine, optionally substituted carboxyl optionally substituted aryl, optionally substituted heteroaryl, optionally substituted carbocyclyl, optionally substituted heterocyclyl as well as selected as any combination thereof.

B) In all of the foregoing aspects wherein the organic molecule is appended with one or more chemical substituents, the chemical substituent(s) preferably are individually and independently selected from the group consisting of halogen, optionally substituted alkyl, optionally substituted amino, optionally substituted carboxyl, optionally substituted aryl, optionally substituted heteroaryl, optionally substituted carbocyclyl, optionally substituted heterocyclyl as well as selected as any combination thereof.

C) In all of the foregoing aspects wherein the organic molecule is appended with one or mare chemical substituents, the one or more appended chemical substituents preferably are individually and independently selected from the group consisting of optionally substituted alkyl, optionally substituted aryl, optionally substituted heteroaryl, optionally substituted carbocyclyl, optionally substituted heterocyclyl as well as selected as any combination thereof.

D) In all of the foregoing aspects wherein the organic molecule is appended with one or more chemical substituents, the one or more appended chemical substituents preferably are individually and independently selected from the group consisting of optionally substituted alkyl, optionally substituted aryl, optionally substituted heteroaryl as well as selected as any combination thereof.

E) In all of the foregoing aspects wherein the organic molecule is appended with one or more chemical substituents, the one or more appended chemical substituents preferably are individually and independently selected from the group consisting of halogen, optionally substituted alkoxy, optionally substituted aliphatic amino, optionally substituted alkyl diamine, optionally substituted aliphatic carboxyl as well as selected as any combination thereof.

F) In all of the foregoing aspects wherein the organic molecule is appended with one or more chemical substituents, the one or more appended chemical substituents preferably are individually and independently selected form the group consisting of optionally substituted aryl, optionally substituted heteroaryl as well as selected as any combination thereof.

G) In all of the foregoing aspects wherein the organic molecule is appended with one or more chemical substituents, the one or more appended chemical substituents number from one to two, more preferably one.

H) In all of the foregoing aspects wherein the organic molecule is appended with one or more chemical substituents, the one or more appended chemical substituents number from one to two, more preferably one.

I) In a further aspect of the invention, the compounds of the above described first and second aspects include the pharmaceutically acceptable salts, the N-oxide derivatives, protected derivatives, individual isomers and mixture of isomers thereof as well as mixtures of these compounds, salts and the like with pharmaceutically acceptable solvents.

Preferably, a subclass of the compounds of the first and second aspects has a bicyclic aromatic structure according to Formula (V):

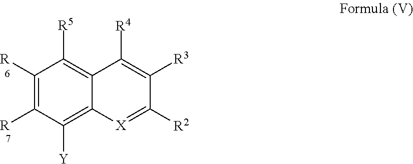

For Formula V, X is nitrogen and Y is selected from the group consisting of O, N, and S. When Y is O or S, Y may be hydroxyl, alkoxy, mercapto or thioalkyl group. When Y is N, it may be a primary, secondary or tertiary amine group. Preferably Y is O or S, more preferably S.

For Formula (V), R², R³, R⁴, R⁵, R⁶, and R⁷ are each independently selected from hydrogen, halogen, CN, optionally substituted carboxyl, optionally substituted ester, optionally substituted amine, optionally substituted amide, optionally substituted thioamide, optionally substituted aliphatic or aryl carboxyl, optionally substituted aliphatic or aryl carboxyamide, optionally substituted aminoalkylamine, carboxyl, ester, alkyl aliphatic ester, amine, optionally substituted aliphatic diamine, optionally substituted aminoalkyl carboxyl, optionally substituted amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs, optionally substituted alkyl, optionally substituted alkenyl, optionally substituted alkynyl, optionally substituted alkoxy, optionally substituted alkthioxy, optionally substituted alkoxalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkyheteroaryl, optionally substituted alkheterocyclyl, optionally substituted carbocyclyl, optionally substituted alkylcarbocyclyl, and any combination thereof. Included as examples of such substituents are carboxyl, ester, alkylcarboxyl, alkyl alkenyl ester, amine, alkylenyl diamine, aminoalkyl carboxyl, amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxyalkylheteroaryl, carboxylalkylcycloalkyl, arylalkoxy, heteroarylakoxy, cycloalkylalkoxy, the corresponding thio analogs. These examples may be optionally substituted by functional groups as given in the DEFINITIONS section. Preferably, the substituents R², R³, R⁴, R⁵, R⁶, and R⁷, may be hydrogen or any of the chemical substituent groups listed above as A) through I) for the organic molecules of the aspects of the invention.

Additionally, the substituents for formula V, i.e., R², R³, R⁴, R⁵, R⁶, and R⁷, may include optionally substituted natural or non-natural peptidyl containing 2 to 6 amino acid moieties, optionally substituted natural or non-natural peptidyl through a linker. The linker is selected from optionally substituted alkyl, optionally substituted alkoxy, optionally substituted amine, optionally substituted carbonyl and optionally substituted carboxylic acid. One, two or three peptide substituents are preferred, one or two are more preferred, two are most preferred. As mentioned above in connection with the first and second embodiments, certain bicyclic aromatic compounds are excluded except when the peptide substituent or substituents are present.

In another feature of the first and second aspects, the disclosed compounds include dimers of Formula V as well as the pharmaceutically acceptable salts, the N-oxide derivatives, protected derivatives, individual isomers and mixture of isomers thereof in addition to mixtures of all of these compounds and forms with pharmaceutically acceptable solvents.

Preferably, a subclass of these dimers has a structure according to Formula (VI):

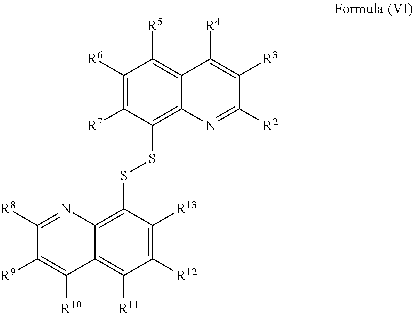

For Formula V1, R2, R3, R4, R5, R6, R7, R8, R9, R10, R11, R12 and R13 are each independently selected from hydrogen, halogen, CN, optionally substituted carboxyl, optionally substituted ester, optionally substituted amine, optionally substituted amide, optionally substituted thioamide, optionally substituted aliphatic or aryl carboxyl, optionally substituted aliphatic or aryl carboxyamide, optionally substituted aminoalkylamine, carboxyl, ester, alkyl aliphatic ester, amine, optionally substituted aliphatic diamine, optionally substituted aminoalkyl carboxyl, optionally substituted amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs, optionally substituted alkyl, optionally substituted alkenyl optionally substituted alkynyl, optionally substituted alkoxy, optionally substituted alkthoxy, optionally substituted alkoxalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkyheteroaryl, optionally substituted alkheterocyclyl, optionally substituted carbocyclyl, optionally substituted alkylcarbocyclyl, and any combination thereof. Included as examples of such substituents are carboxyl, ester, alkylcarboxyl, alkyl alkenyl ester, amine, alkylenyl diamine, aminoalkyl carboxyl, amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs. These examples may be optionally substituted by functional groups as given in the DEFINITIONS section. Preferably, the substituents R2, R3, R4, R5, R6, R7, R8, R9, R10, R11, R12 and R13 may be hydrogen or any of the chemical substituent groups listed above as A) through I) for the organic molecules of the aspects of the invention.

Additionally, the substituents for formula V, i.e., R2, R3, R4, R5, R6, R7, R8, R9, R10, R11, R12 and R13 may include optionally substituted natural or non-natural peptidyl containing 2˜6 amino acids moiety, optionally substituted natural or non-natural peptidyl through a linker and the linker is selected from optionally substituted alkyl optionally substituted alkoxy, optionally substituted amine, optionally substituted carbonyl and optionally substituted, carboxylic acid. One or two peptide substituents are preferred. As mentioned above for the first and second aspects, the dimers of certain bicyclic aromatic compounds are excluded except when the peptide substituent or substituents are present.

In another feature, the third and fourth aspects include pyran-like compounds as well as their pharmaceutically acceptable salts, the N-oxide derivatives, protected derivatives, individual isomers and mixture of isomers thereof and mixtures with pharmaceutically acceptable solvents in addition to mixtures of the pyran-like compounds and their forms with pharmaceutically acceptable solvents.

Preferably, a subclass of these pyran-like compounds has a structure according to Formula (VII):

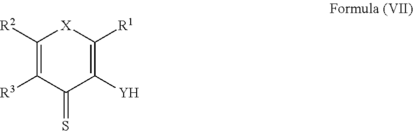

For Formula VII, X, Y are independently selected from the group consisting of O, N, S. Preferably, X is O or S and Y is N, more preferably, X is S and Y is N.

R1, R2 and R3 are each independently selected from hydrogen, halogen, CN, optionally substituted carboxyl, optionally substituted ester, optionally substituted amine, optionally substituted amide, optionally substituted thioamide, optionally substituted aliphatic or aryl carboxyl, optionally substituted aliphatic or aryl carboxyamide, optionally substituted aminoalkylamine, carboxyl, ester, alkyl aliphatic ester, amine, optionally substituted aliphatic diamine, optionally substituted aminoalkyl carboxyl, optionally substituted amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs, optionally substituted alkyl, optionally substituted alkenyl, optionally substituted alkynyl, optionally substituted alkoxy, optionally substituted alkthioxy, optionally substituted alkoxalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkyheteroaryl, optionally substituted alkheterocyclyl, optionally substituted carbocyclyl, optionally substituted alkylcarbocyclyl, and any combination thereof. Included as examples of such substituents are carboxyl, ester, alkylcarboxyl, alkyl alkenyl ester, amine, alkylenyl diamine, aminoalkyl carboxyl, amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs. These examples may be optionally substituted by functional groups as given in the DEFINITIONS section. Preferably, the substituents R1, R2 and R3 may be hydrogen or any of the chemical substituent groups listed above as A) through I) for the organic molecules of the aspects of the invention.

Additionally, the substituents for formula VII may optionally include optionally substituted natural or non-natural, peptidyl containing 2 to 6 amino acids moiety, optionally substituted natural or non-natural peptidyl through, a linker and the linker is selected from optionally substituted alkyl, optionally substituted alkoxy, optionally substituted amine, optionally substituted carbonyl and optionally substituted carboxylic acid. One, two or three peptide substituents are preferred. As mentioned above for the third and fourth embodiments, certain pyran-like compounds are excluded except when the peptide substituent or substituents are present.

In a further feature, the fifth aspect includes ketones as well as their pharmaceutically acceptable salts, the N-oxide derivatives, where applicable, protected derivatives, individual isomers and mixture of isomers thereof, and mixtures with pharmaceutically acceptable solvents in addition to mixtures of these ketones and the like with pharmaceutically acceptable solvents.

Preferably, a subclass of these ketones has a structure according to Formula (VIII):

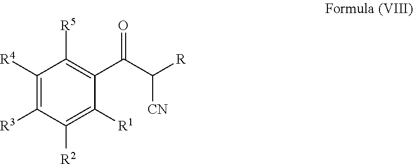

For Formula VIII, R, R1, R2, R3, R4 and R5 are each independently selected from hydrogen, halogen, CN, optionally substituted carboxyl, optionally substituted ester, optionally substituted amine, optionally substituted amide, optionally substituted thioamide, optionally substituted aliphatic or aryl carboxyl, optionally substituted aliphatic or aryl carboxyamide, optionally substituted aminoalkylamine, carboxyl, ester, alkyl aliphatic ester, amine, optionally substituted aliphatic diamine, optionally substituted aminoalkyl carboxyl, optionally substituted amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs, optionally substituted alkyl, optionally substituted alkenyl, optionally substituted alkynyl, optionally substituted alkoxy, optionally substituted alkthioxy, optionally substituted alkoxalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkyheteroaryl, optionally substituted alkheterocyclyl, optionally substituted carbocyclyl, optionally substituted alkylcarbocyclyl, and any combination thereof. Included as examples of such substituents are carboxyl, ester, alkylcarboxyl, alkyl alkenyl ester, amine, alkylenyl diamine, aminoalkyl carboxyl, amino alkyl ester, amino alkyl amide, halogen, alkyl halogen, alkylheterocycle, alkylaryl, alkylheteroaryl, aminoalkylaryl, aminoalkylheteroaryl, carboxyalkylaryl, carboxylalkylheteroaryl, carboxylalkylcycloalkyl, arylalkoxy, heteroarylalkoxy, cycloalkylalkoxy, the corresponding thio analogs. These examples may be optionally substituted by functional groups as given is the DEFINITIONS section. Preferably, the substituents R, R1, R2, R3, R4 and R5 may be hydrogen or any of the chemical substituent groups listed above as A) through I) for the organic molecules of the aspects of the invention.

Additionally the substituents for formula VIII optionally may include optionally substituted natural or non-natural peptidyl containing 2˜6 amino acids moiety, optionally substituted natural or non-natural peptidyl through a linker and the linker is selected from optionally substituted alkyl, optionally substituted alkoxy, optionally substituted amine, optionally substituted carbonyl and optionally substituted, carboxylic acid. One or two peptide substituents are preferred. As mentioned above for the fifth embodiment, certain ketone compounds are excluded except when the peptide substituent or substituents are present.

### EXAMPLES OF PEPTIDE SUBSTITUTED COMPOUNDS

All aspects, embodiments and features of the invention include optional peptide substituents appended to the framework of the organic molecule. The presence of the peptide substituents enables selectivity directed activity of the inhibitory compounds of the invention. The peptides may be selected as substrates for the JAMM domain targeted or may be the selective peptide moiety of an antibody, such as a monoclonal antibody, that is prepared using the JAMM domain or its corresponding protein as an antigenic material.

Examples of such peptide substituted compounds include the following.

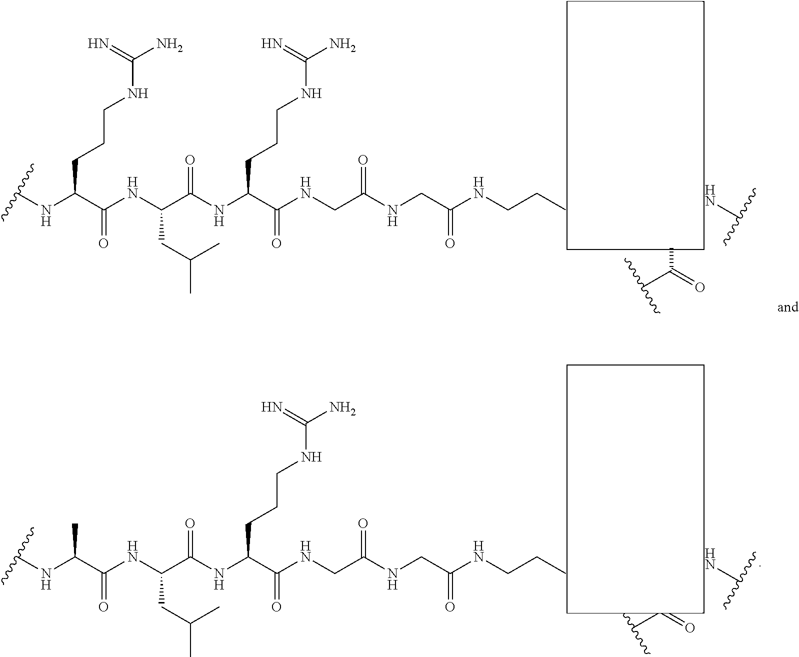

In various embodiments, a backbone will have homology to the above sequences, such as greater than 50%, 60%, 70%, 80%, or 90% identity with the above sequences.

### Pharmaceutical Compositions

A therapeutically effective amount of a compound of the present invention will depend upon a number of factors including, for example, the age and weight of the subject being treated, the precise condition requiring treatment and its severity, the nature of the formulation, and the route of administration, and will ultimately be at the discretion of the attendant physician or veterinarian. In this aspect of the invention, a pharmaceutical formulation, comprises a pharmaceutically acceptable carrier and an amount of a compound of any foregoing embodiments under the COMPOUNDS section effective to inhibit the metalloprotease activity of a JAMM metalloprotease containing protein.

However, an effective amount of a compound of the invention for the treatment of diseases or conditions associated with inappropriate JAMM activity will generally be in the range of 0.1 mg to 100 mg/kg body weight of recipient (mammal) per day and more usually in the range of 1 mg to 10 mg/kg body weight per day. This amount may be given in a single dose per day or in a number (such as two, three, four, five or six) of sub-doses per day such that the total daily dose is the same. An effective amount of a salt or solvate, thereof, may be determined as a proportion of the effective amount of the compound of the invention per se.

The compounds of the present invention may be in the form of and/or may be administered as pharmaceutically acceptable salts, N-oxide derivatives, protected derivatives (i.e. prodrugs), and individual isomers and mixture of isomers thereof. Typically, the salts of the present invention are pharmaceutically acceptable salts. Salts encompassed within the term “pharmaceutically acceptable salts” refer to non-toxic salts of the compounds of this invention. Suitable pharmaceutically acceptable salts can include acid or base additions salts.

A pharmaceutically acceptable acid addition salt can be formed by reaction of a compound of with a suitable inorganic or organic acid (such as hydrobromic, hydrochloric, sulfuric, nitric, phosphoric, succinic, maleic, formic, acetic, propionic, fumaric, citric, tartaric, lactic, benzoic, salicylic, glutamaic, aspartic, p-toluenesulfonic, benzenesulfonic, methanesulfonic, ethanesulfonic, naphthalenesulfonic, or hexanoic acid), optionally in a suitable solvent such as water or an organic solvent, to give the salt which is usually isolated for example by crystallization and filtration. A pharmaceutically acceptable acid addition salt of a compound can comprise, for example, a hydrobromide, hydrochloride, sulfate, nitrate, phosphate, succinate, maleate, formate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate, or hexanoate salt.

A pharmaceutically acceptable base addition salt may, where there is a suitable acidic group, be formed by reaction of a compound with a suitable inorganic or organic base (e.g. triethylamine, ethanolamine, triethanolamine, choline, arginine, lysine or histidine), optionally in a suitable solvent such as water or an organic solvent, to give the base addition salt which is usually isolated for example by crystallization and filtration.

Other suitable pharmaceutically acceptable salts include pharmaceutically acceptable metal salts, for example pharmaceutically acceptable alkali-metal or alkaline-earth-metal salts such as sodium, potassium, calcium or magnesium salts. Other salts, e.g. oxalates or trifluoroacetates, may be used, for example in the isolation of compounds of the invention, and are included within the scope of this invention.

The invention includes within its scope all possible stoichiometric and non-stoichiometric forms of the compounds of the invention.

While it is possible that, for use in therapy, a compound the invention, as well as salts or solvates thereof, may be administered as the raw chemical, it is possible to present the active ingredient as a pharmaceutical composition. Accordingly, the invention further provides a pharmaceutical composition, which comprises a compound of the invention and salts or solvates thereof, and one or more pharmaceutically acceptable carriers, diluents, or excipients. The compounds and salts or solvates thereof, are as described above. The carriers, diluents, or excipients must be acceptable in the sense of being compatible with the other ingredients of the formulation and not deleterious to the recipient thereof. In accordance with another aspect of the invention there is also provided a process for the preparation of a pharmaceutical composition including admixing a compound disclosed above, or salts, solvates and physiological functional derivatives thereof (i.e., prodrugs), with one or more pharmaceutically acceptable carriers, diluents or excipients.

Pharmaceutical compositions comprising compounds of the invention may be presented in unit dose forms containing a predetermined amount of active ingredient per unit dose. Such a unit may contain, for example, 5 mg to 1 g, 1 mg to 700 mg, or 5 mg to 100 mg, of a compound of the invention depending on the condition being treated, the route of administration and the age, weight and condition of the patient. Such unit doses may therefore be administered more than once a day. In one embodiment unit dosage compositions are those containing a daily dose or sub-dose (for administration more than once a day), as herein above recited, or an appropriate fraction thereof of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, inhaled, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association the active ingredient with one or more carriers or excipients.

Pharmaceutical compositions adapted for oral administration may be presented as discrete units such as pills, capsules or tablets; powders or granules; solutions or suspensions in aqueous or non-aqueous liquids; edible foams or whips; or oil-in-water liquid emulsions or water-in-oil liquid emulsions. For instance, for oral administration in the form of a pill, tablet or capsule, the active drug component can be combined with an oral, non-toxic pharmaceutically acceptable inert carrier such as ethanol glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium, stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, or solutions of cellulosic or polymeric materials and forcing through a screen. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added so these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The formulation can also be prepared to prolong or sustain the release as for example by coating or embedding particulate material in polymers, wax or the like.

The compounds of the invention and salts and thereof may also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The disclosed compounds may be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Remington: The Science and Practice of Pharmacy, 21^{st} Edition, hereby incorporated by reference in its entirety.

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or as enemas. Pharmaceutical compositions adapted for vaginal administration may be presented as pessaries, tampons, creams, gels, pastes, foams or spray formulations.

Pharmaceutical compositions for nasal or inhaled administration wherein the carrier is a solid include a coarse powder having a particle size for example in the range 20 to 500 microns which is administered by rapid inhalation through the nasal passage from a container of the power held close up to the nose. Pharmaceutical compositions adapted for administration by inhalation include line particle dusts or mists, which may be generated by means of various types of metered, dose pressurised aerosols, nebulizers or insufflators. Suitable compositions wherein the carrier is a liquid for administration as a nasal spray or as nasal drops, include aqueous or oil solutions of the active ingredient.

Pharmaceutical compositions adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents, The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the compositions may include other agents conventional in the art having regard to the type of formulation in question, for example those suitable for oral administration may include flavouring agents.

### Pharmaceutical Combinations

The compounds of the present invention and their salts and solvates, thereof, may be employed alone or in combination with other therapeutic agents for the treatment of the diseases or conditions associated with inappropriate JAMM activity, for instance cancer.

In particular, combination with at least one other anti-cancer therapy is envisaged. In particular, in anti-cancer therapy, combination with other chemotherapeutic, hormonal or antibody agent is envisaged as well as combination with surgical therapy and radiotherapy. Combination-therapies according to the present invention thus comprise the administration of at least one compound of the invention or a pharmaceutically acceptable salt or solvate thereof, and the use of at least one other cancer treatment method. In one embodiment combination therapies according to the present invention comprise the administration of at least one compound of the invention or a pharmaceutically acceptable salt or solvate thereof, or a physiologically functional derivative thereof, and at least one other pharmaceutically active agent, for example an anti-neoplastic agent. A compound of the invention and the other pharmaceutically active agents may be administered together or separately and, when administered separately this may occur simultaneously or sequentially in any order and by any convenient route. The amounts of the compound of the invention and the other pharmaceutically active agents and the relative timings of administration will be selected in order to achieve the desired combined therapeutic effect.

In one embodiment, another anti-cancer therapy is at least one additional chemotherapeutic therapy. Such chemotherapeutic therapy may include one or more of the following categories of anti-cancer agents:
antiproliferative/antineoplastie drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptochecin);
cytostatic agents such as antioestrogens (for example tamoxifen, toremifine, raloxifine, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate) aromatase inhibitors (for example as anastrozole, letrozole, vorazole and exemestane) and inhibitors of 5a-reductase such as finasteride;
agents which inhibit cancer cell invasion (for example metalloproteinase inhibitors and inhibitors of urokinase plasminogen activator receptor function);
inhibitors of growth factor function, for example such inhibitors include growth factor antibodies, growth factor receptor antibodies (for example the anti-erbb2 antibody trastuzumab (Herceptin™) and the anti-erbb1 antibody cetuximab (C225), farnesyl transferase inhibitors, tyrosine kinase inhibitors and serine-threonine kinase inhibitors, for example inhibitors of the epidermal growth factor family (for example EGFR family tyrosine kinase inhibitors such as N-(3-chloro-4-fluorophenyl-7-methoxy-6-(3-morpholinoproproxy)quinazolin-4-amine (gefitinib, AZD1839), N-3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazol-in-4-amine (CI-1033), for example inhibitors of the platelet-derived growth factor family and for example inhibitors of the hepatocyte growth factor family;
antiangiogenic agents such as those which inhibit the effects of vascular endothelial growth factor, (for example the anti-vascular endothelial cell growth factor antibody bevacizumab (Avastin™), and compounds that work by other mechanisms (for example linomide, inhibitors of integrin avb3 function and angiostatin);
gene therapy approaches, including for example approaches to replace aberrant genes such as aberrant p53 or aberrant BRCA1 or BRCA2, GDEPT (gene-derived enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
immunotherapy approaches, including for example ex-vivo and in-vivo approaches to increase the immunogenecity of patient tumour cells, such as transfection with cytokines such as interleukin-2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies.

In one particular embodiment compositions of the invention are used in combination with the proteasome inhibitor MG 132 (see Banerjee and Liefshitz (2001), Potential of the proteasome inhibitor MG-132 as an anticancer agent, alone and in combination. Anticancer Res. 21 3941). In another embodiment compositions of the invention are used in combination with TRAIL or a TRAIL receptor agonist, for instance with MD5-1. In another embodiment the compositions of the invention are used in combination with bortezomib, or other proteasome inhibitors.

When a compound of the invention is used in combination with a second therapeutic agent active against the same disease, the dose of each compound may differ from that when the compound is used alone. In various embodiments, synergistic combinations are envisioned.

### Screening Methods for Identification of Anti-JAMM Compounds

The invention is also directed to screening methods for identification of compounds that will inhibit, ameliorate or otherwise diminish the enzymatic activity of the JAMM domain. These compounds constitute leads for development of Anti-JAMM candidates as well as such candidates themselves. The method includes the use of a standardized ubiquinated or neddylated protein or peptide substrate for a JAMM enzyme. The standardized substrate is tagged with a detectable moiety that will enable differentiation between (a) the substrate having the ubiquitin or ubiquitin chain or Nedd8 moiety and (b) the cleaved substrate missing all or part of the ubiquitin or ubiquitin chain or Nedd8 moiety. In particular, the method for screening for a compound that inhibits the metalloprotease activity of a protein containing a JAMM metalloprotease domain includes the following steps.

The first step involves selecting a candidate from a group of organic molecules that could possibly be inhibitory of the JAMM domain.

The second step involves testing the selected candidate in a JAMM domain inhibition assay.

The testing steps include a first substep of combining a JAMM enzymatic material selected from the group consisting of a JAMM domain containing protein, a signalosome complex and a 26S proteasome complex containing the JAMM protein, and a protein substrate selected from the group consisting of a protein modified by a ubiquitin, a protein modified by a ubiquitin-like modifier and a protein modified by a ubiquitin chain to produce an enzymatic medium wherein the protein substrate is modified with a tag that is detectable by measurement of molecular weight, spectroscopic interaction or cinematographic R_{f} determination.

The second substep includes conducting a first measurement of the enzymatic medium relative to the protein substrate alone wherein the first measurement is made by a detection of the tag.

The third substep includes combining the selected candidate with the protein substrate and adding the JAMM enzymatic material to produce a candidate medium.

The fourth substep includes conducting a second measurement of the candidate medium relative to the protein substrate alone wherein the second measurement is made by detection of the tag.

The fifth step includes comparing the first and second measurements to identify a candidate that demonstrates at least about a 50% inhibition at a concentration of no more than 500 micromolar in the candidate medium, the difference between the first and second measurements being at least about 50% with the second measurement being greater than the first measurement.

Screening methods are also provided for measuring the activity of any test agent on CSN that involves monitoring the effect of the test agent on the ability of CSN to deconjugate Nedd8 from a cullin subunit (chosen from the set of Cu11, Cu12, Cu13, Cu14a, Cu14b, Cu15, Cu17, and PARC) of a cullin-RING ubiquitin ligase (CRL). In various embodiments, the method involves contacting the Nedd8-conjugated cullin with CSN in the presence or absence of the test agent.

In one aspect, such a method comprises modifying Nedd8 with a fluorescent molecule and cleaving the fluorescent Nedd8-cullin conjugate. The course of the cleavage reaction is monitored by a decrease in fluorescence polarization of the fluorescent molecule.

In one aspect, such a method involves modification of Nedd8 and the cullin or RING subunit with fluorescent dyes that undergo fluorescence resonance energy transfer (FRET). Cleavage of the conjugate is monitored by loss of FRET signal (i.e., reduced fluorescence of the acceptor dye or dequenching of the donor dye).

In one aspect, the chosen assay method is used to screen a library of small molecules to identify those that inhibit the deconjugating activity of CSN.

In one aspect, cleavage of labeled Nedd8 from cullin is monitored by sodium dodecylsulfate-polyacrylamide gel electrophoresis followed by detection, of either the Cull or Nedd8 polypeptide.

Compounds that may be selected as candidates for this screening method include those described above under the COMPOUNDS section.

In particular, the method for screening will characterize compounds identified above in the COMPOUNDS section wherein the one or more appended chemical substituents are selected from the group consisting of halogen, optionally substituted alkyl, optionally substituted alkoxy, optionally substituted alkthioxy, optionally substituted amino, optionally substituted carboxyl, optionally substituted aryl, optionally substituted heteroaryl, optionally substituted carbocyclyl, optionally substituted heterocyclyl and any combination thereof.

Preferred screening characterizes such compounds wherein the one or more appended chemical substituents are selected from the group consisting of halogen, optionally substituted alkyl, optionally substituted amino, optionally substituted carboxyl, optionally substituted aryl, optionally substituted heteroaryl, optionally substituted carbocyclyl, optionally substituted heterocyclyl and any combination thereof.

Especially preferred screening characterizes such compounds wherein the one or more appended chemical substituents are selected from the group consisting of optionally substituted alkyl, optionally substituted aryl, optionally substituted heteroaryl, optionally substituted carbocyclyl, optionally substituted heterocyclyl and any combination thereof.

Also especially preferred screening characterizes such compounds wherein the one or more appended chemical substituents are selected from the group consisting of optionally substituted alkyl optionally substituted aryl, optionally substituted heteroaryl and any combination thereof.

Especially preferred also includes screening to identify compounds wherein the one or more appended chemical substituents are selected from the group consisting of halogen, optionally substituted alkoxy, optionally substituted amino, optionally substituted carboxyl and any combination thereof.

Also especially preferred screening includes compounds wherein the one or more appended chemical substituents are selected form the group consisting of optionally substituted aryl, optionally substituted heteroaryl and any combination thereof; the one or more appended chemical substituents numbers from one to four; and wherein the one or more appended chemical substituents numbers from one to two.

### Methods of Use

Treatment of Disease

The compounds of the present invention and their salts and solvates, thereof, may be employed alone or in combination with other therapeutic agents for the treatment of the diseases or conditions associated with inappropriate JAMM activity.

In various embodiments, compounds of the invention may be used to treat neoplastic growth, angiogenesis, infection, inflammation, immune-related diseases, ischemia and reperfusion injury, multiple sclerosis, rheumatoid arthritis, neurodegenerative conditions, or psoriasis.

Neoplastic growth may include cancer. Suitably, the present invention relates to a method for treating or lessening the severity of a cancer selected from: brain (gliomas), glioblastomas, breast, Wilm's tumor, Ewing's sarcoma, rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T cell leukemia, chronic myelogenous leukemia, chronic lymphocytic leukemia, Hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor) and testicular cancer. In various embodiments, the cancer is selected from brain cancer (gliomas), glioblastomas, breast cancer, colon cancer, head and/neck cancer, kidney cancer, lung cancer, liver cancer, melanoma, ovarian cancer, pancreatic cancer, prostate cancer, sarcoma and thyroid cancer.

In various embodiments, the cancer to be treated is associated with the proteasome. See Voorhees et al., The Proteasome as a Target for Cancer Therapy, Clinical Cancer Research, vol. 9, 6316-6325, December 2003, incorporated by reference in its entirety. In various embodiments, the cancer is associated with a particular target, such as NFkB, p 44/42 MAPK. P-gp, TopI, TopIIalpha.

in various embodiments, the cancer is a solid tumor. In various embodiments, the cancer is selected from multiple myeloma, metastatic breast cancer, non-small cell lung cancer, prostate cancer, advanced colorectal cancer, ovarian or primary peritoneal carcinoma, hormone refectory prostate cancer, squamous cell carcinoma of the head and neck, metastatic pancreatic adenocarcinoma, gastroesophageal junction or stomach, or non-Hodgkin's lymphoma.

A method of using the compounds described herein for treating a disorder characterized by an inappropriate level of CRL or proteasome activity, or in which a reduction of the normal level of CRL or proteasome activity yields a clinical benefit. This disorder can include cancer or immune disorders characterized by excessive cell proliferation or cellular signaling. Among cancers, this includes human cancers that overexpress c-Myc or express an oncogenic form of the K-Ras protein.

Neurodegenerative diseases and conditions may include without limitation stroke, ischemic damage to the nervous system, neural trauma (e.g., percussive brain damage, spinal cord injury, and traumatic damage to the nervous system), multiple sclerosis and other immune-mediated neuropathies (e.g., Guillain-Barre syndrome and its variants, acute motor axonal neuropathy, acute inflammatory demyelinating polyneuropathy, and Fisher Syndrome), HIV/AIDS dementia complex, axonomy, diabetic neuropathy, Parkinson's disease, Huntington's disease, ALS, multiple sclerosis, bacterial, parasitic, fungal, and viral meningitis, encephalitis, vascular dementia, multi-infarct dementia, Lewy body dementia, frontal lobe dementia such as Pick's disease, subcortical dementias, (such as Huntington or progressive supranuclear palsy), local cortical atrophy syndromes (sues as primary aphasia), metabolic-toxic dementias (such as chronic hypothyroidism or B12 deficiency), and dementias caused by infections (such as syphilis or chronic meningitis). Compounds of the invention may be used to treat Alzheimer's disease, including administering to a subject an effective amount of an agent or composition (e.g., pharmaceutical composition) disclosed herein.

Compounds of the invention may be used to treat cachexia and muscle-wasting diseases. Compounds of the invention may be used to treat such conditions wherein the condition is related to cancer, chronic infectious diseases, fever, muscle disuse (atrophy) and denervation, nerve injury, fasting, renal failure associated with acidosis, diabetes, and hepatic failure.

Compounds of the invention can be used to treat hyperproliferative conditions such as diabetic retinopathy, macular degeneration, diabetic nephropathy, glomerulosclerosis, IgA nephropathy, cirrhosis, biliary atresia, congestive heart failure, scleroderma, radiation-induced fibrosis, and lung fibrosis (idiopathic pulmonary fibrosis, collagen vascular disease, sarcoidosis, interstitial lung diseases and extrinsic lung disorders). The treatment of burn victims is often hampered by fibrosis, thus, an additional embodiment of the application is the topical or systemic administration of the inhibitors to treat burns. Wound closure following surgery is often associated with disfiguring scars, which may be prevented by inhibition of fibrosis. Thus, in certain embodiments, the application relates to a method for the prevention or reduction of scarring.

Compounds of the invention can be used to treat ischemic conditions or reperfusion injury for example acute coronary syndrome (vulnerable plaques), arterial occlusive disease (cardiac, cerebral, peripheral arterial and vascular occlusions), atherosclerosis (coronary sclerosis, coronary artery disease), infarctions, heart failure, pancreatitis, myocardial hypertrophy, stenosis, and restenosis.

Compounds of the invention can be used for the inhibition of TNFalpha to prevent and/or treat septic shock.

Compounds of the invention can be used for inhibiting antigen presentation in a cell, including exposing the cell to an agent described herein. A compound of the invention may be used to treat immune-related conditions such as allergy, asthma, organ/tissue rejection (graft-versus-host disease), and auto-immune diseases, including, but not limited to, lupus, rheumatoid arthritis, psoriasis, multiple sclerosis, and inflammatory bowel diseases (such as ulcerative colitis and Crohn's disease). Thus, a further embodiment is a method for moedulating the immune system of a subject (e.g., inhibiting transplant rejection, allergies, auto-immune diseases, and asthma), including administering to the subject an effective amount of a compound of the invention.

Compounds of the invention can be used in methods for altering the repertoire of antigenic peptides produced by the proteasome or other protein assembly with multicatalytic activity.

Compounds of the invention can be used in methods for inhibiting IKB-alpha degradation, including contacting the cell with an agent identified herein. A further embodiment is a method for reducing the cellular content of NF-KB in a cell, muscle, organ, or subject, including contacting the cell, muscle, organ, or subject with a compound of the invention.

Compounds of the invention can be used in methods for affecting cyclin-dependent eukaryotic cell cycles. Compounds of the invention can be used in methods for treating a proliferative disease in a subject (e.g., cancer, psoriasis, or restenosis). Compounds of the invention can be used for treating cyclin-related inflammation in a subject.

One embodiment is a method for treating p53-related apoptosis, including administering to a subject an effective amount of a compound of the invention.

In another embodiment, the agents of the present application are useful for the treatment of a parasitic infection, such as infections caused by protozoan, parasites. In certain such embodiments, the agents are useful for the treatment of parasitic infections in humans caused by a protozoan parasite selected from Plasmodium sps., Trypanosoma sps., Leishmania sps., Pneumocystis carinii, Toxoplasma gondii, Entamoeba histolytica, Entamoeba invadens, and Giardia lamblia. In certain embodiments, the agents are useful for the treatment of parasitic infections in animals and livestock caused by a protozoan parasite selected from Plasmodium hermani, Cryptosporidium sps., Echinococcus granulosus, Eimeria tenella, Sarcocystis neurona, and Neurospora crassa. Other compounds useful as proteasome inhibitors in the treatment of parasitic diseases are described in WO 98/10779, which is incorporated herein in its entirety.

In particular, the methods of treatment include inhibiting, arresting, ameliorating, minimizing and/or eliminating malconditions associated with the inability of cells to metabolize, degrade or otherwise remove ubiquitin tagged proteins and peptides because the tag has been cleaved, degraded, removed or otherwise rendered disfunctional as a result of JAMM metalloprotease domain activity. Included are methods in which a human disorder characterized by abnormal regulatory peptide degradation resulting in excessive cell proliferation or cell signaling. The methods are directed to administration of an effective amount of a compound or pharmaceutical formulation disclosed above so that the abnormal regulatory peptide degradation is ameliorated, reduced or inhibited. In particular, the human disorders include a cancer or immune disorder, a cancer resulting from overexpression of c-Myc or expression of an oncogenic form of the K-Ras protein. The methods also include inhibition or amelioration of JAMM metalloprotease domain activity in a human patient suffering from abnormal JAMM metalloprotease domain activity on ubiquitin modified proteins. As described above, these methods involve administering to the patient an effective amount of a compound or pharmaceutical formulation disclosed above so that the abnormal JAMM metalloprotease domain activity is ameliorated, reduced or inhibited.

Diagnostics

Various cellular proteins are subjected to proteolytic processing during maturation or activation. The compositions identified herein can also be useful as diagnostic agents (e.g., in diagnostic kits or for use in clinical laboratories) for screening for proteins (e.g., enzymes, transcription factors) processed by Ntn hydrolases, including the proteasome. The agents are also useful as research reagents for specifically binding the X/MB 1 subunit or alpha-chain and inhibiting the proteolytic activities associated with it. For example, the activity of (and specific inhibitors of) other subunits of the proteasome can be determined.

Inhibitors identified herein can be used to determine whether a cellular, developmental, or physiological process or output is regulated by proteolytic activity. One such method includes obtaining an organism, an intact cell preparation, or a cell extract; exposing the organism, cell preparation, or cell extract to an agent identified herein; exposing the agent-exposed organism, cell preparation, or cell extract to a signal, and monitoring the process or output. See, for example, U.S. Pat. No. 7,741,432.

The compounds of this invention may used as a part of a diagnostic assay. For instance cells from a patient may be obtained and an assay may be performed to determine whether the compounds of the invention are likely to be effective therapeutic compounds for that patient. The cells obtained from the patient can be for instance cancerous cells from a tumor. The cells can be cultured and compounds of the invention can be applied to determine how the cancerous cells respond.

The Diagnostics aspect of the invention also includes an assay for the determination of inhibition of JAMM metalloprotease domain activity. The assay involves combining a JAMM enzymatic material with a protein substrate and determining whether a potential inhibitory candidate will function in this assay to lessen the enzymatic activity. The JAMM enzymatic material is either a standard or taken from a patient's cells. The protein substrate similarly is either standard or taken from a patient's cells. In particular, the a JAMM enzymatic material selected from the group consisting of a JAMM domain containing protein, a signalosome complex and a 26S proteasome complex containing the JAMM protein that can be isolated from a patient's cells.

In particular, the protein substrate is selected from the group consisting of a protein modified by a ubiquitin, a protein modified by a ubiquitin-like modifier and a protein modified by a ubiquitin chain that can be isolated from a patient's cells. The combination of the JAMM enzymatic material and the protein substrate produces an enzymatic medium. For this medium, the protein substrate is modified with a tag that is detectable by measurement of molecular weight, spectroscopic interaction or chromatographic R_{f} determination,

Following the isolation and tagging, the enzymatic medium is manipulated to conduct a first measurement of the enzymatic medium relative to the protein substrate alone wherein the first measurement is made by a detection of the tag.

Following the first measurement procedure, a potential inhibitory candidate is combined with the tagged protein substrate and the JAMM enzymatic material is added to produce a candidate medium.

The candidate medium is manipulated to conduct a second measurement of the candidate medium relative to the protein substrate alone wherein the second measurement is made by detection of the tag.

Finally, the ability of the inhibitory candidate to be effective treatment for the patient in need is assessed by comparing the first and second measurements to identify a candidate that demonstrates at least about a 50% inhibition at a concentration of no more than 500 micromolar in the candidate medium, the difference between the first and second measurements being at least about 50% with the second measurement being greater than the first measurement.

### ADDITIONAL EMBODIMENTS OF THE COMPOUNDS OF THE INVENTION

Additional embodiments of the compounds of the invention include the following chemical substituents appended to the following aromatic, aliphatic and aliaromatic frameworks. The number indicates the optional positions for substitution.
a) 8-quinolinethiol framework, positions 2, 3, 4, 5, 6, 7,
b) 2-mercaptomethylpyridine framework, positions 2, 3, 4, 5,
c) 5,6,7,8-tetrahedron-8-quinolinethiol framework, positions 2, 3, 4, 5, 6, 7,
d) 1 -mercapto-4-aza-2,3-dihydroindane framework, positions 2, 3, 5, 6, 7,
e) 4-mercaptoindole framework, positions 2, 3, 5, 6, 7,
f) 4-mercaptobenzoimidazole framework, positions 2, 5, 6, 7,
g) 4-mercaptobenzoxazole framework, positions 2, 5, 6, 7,
h) 4-mercaptobenzthiazole framework, positions 2, 5, 6, 7,
i) 8-mercaptoquinazoline framework, positions 2, 6, 7, 8,
j) 3-hydroxy-4H-pyran-4-thione framework, positions 2, 5, 6,
k) 8-mercapto-4H-quinazolin-4-one framework, positions 2, 6, 7, 8,
l) 8-mercapto-2H-4H-quinazolin-2,4-dione framework, positions 6, 7, 8,
m) 3-mercaptopyridinothiophene framework, positions 2, 3, 5, 6, 7
n) a 5 membered single ring framework with X—C—C—Y of aromatic character, or of aliphatic character, or of aliaromatic character, or any combination thereof as defined in the summary of the invention, positions 2, 3, 4, 5
o) a 6 membered single ring framework with X—C—C—Y of aromatic character, or of aliphatic character, or of aliaromatic character, or any combination thereof as defined in the summary of the invention, positions 2, 3, 4, 5, 6,
p) a 5:5 membered bicyclic ring framework with X—C—C—Y of aromatic character, or aliphatic character or of aliaromatic character or any combination thereof as defined in the summary of the invention, positions 2, 3, 4, 5, 6, 7, 8,
q) a 5:6 or a 6:5 membered bicyclic ring framework with X—C—C—Y of aromatic character, or of aliphatic character or of aliaromatic character or any combination thereof as defined in the summary of the invention, positions 2, 3, 4, 5, 6, 7, 8, 9
r) a 6:6 membered bicyclic ring framework with X—C—C—Y of aromatic character, or of aliphatic character or of aliaromatic character or any combination thereof as defined in the summary of the invention, positions 2, 3, 4, 5, 6, 7, 8, 9,

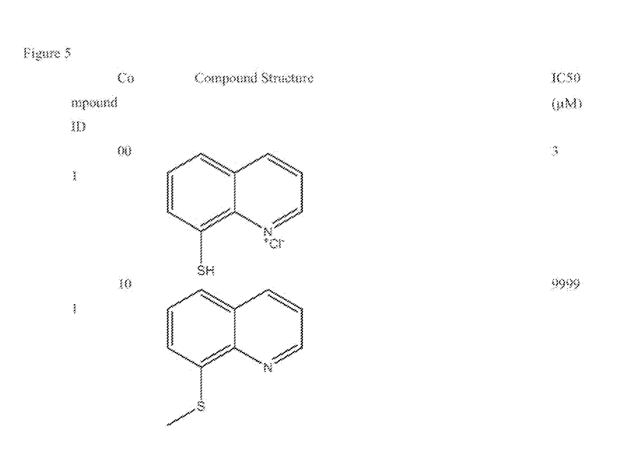

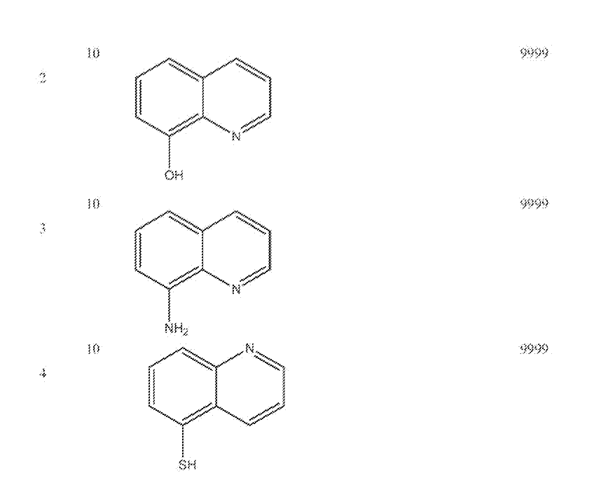

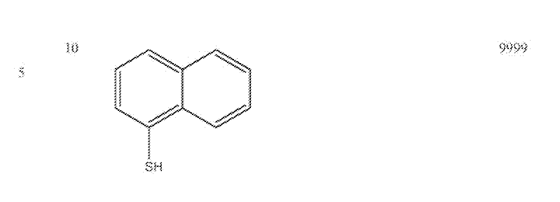

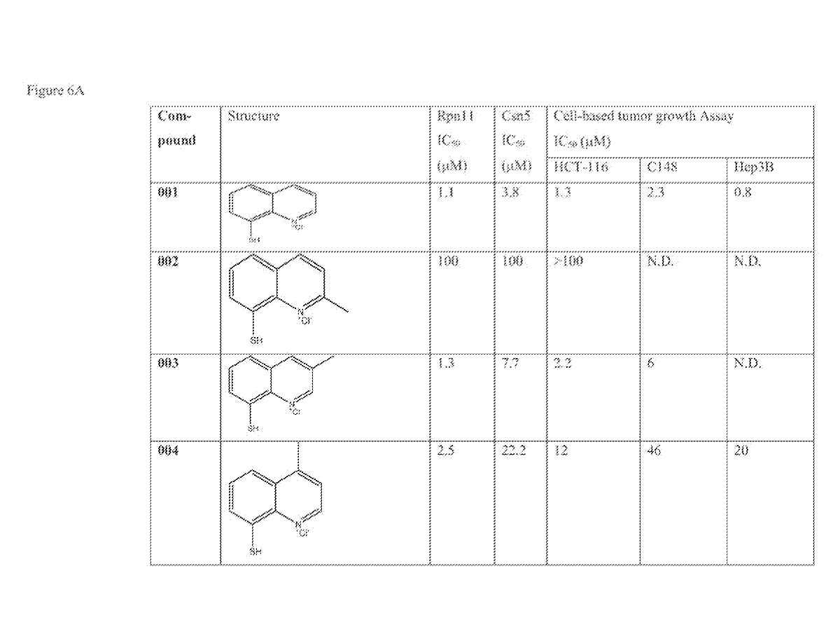

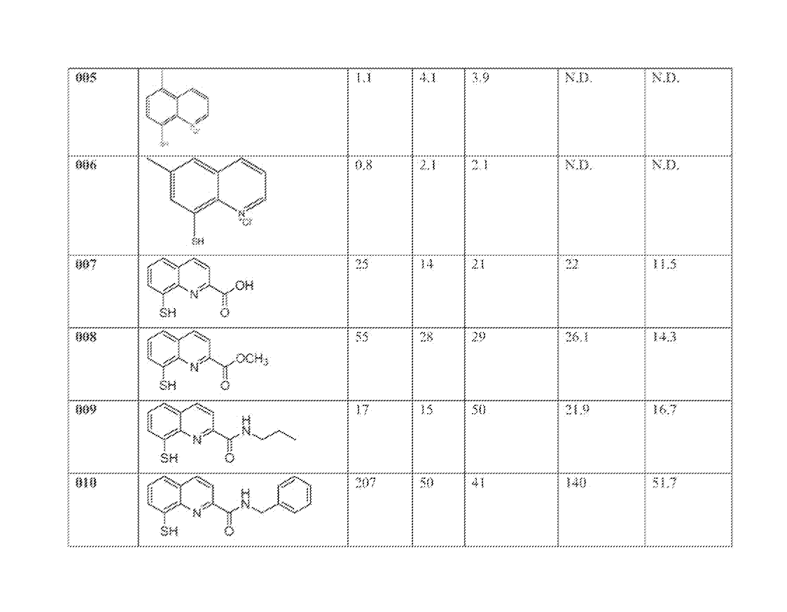

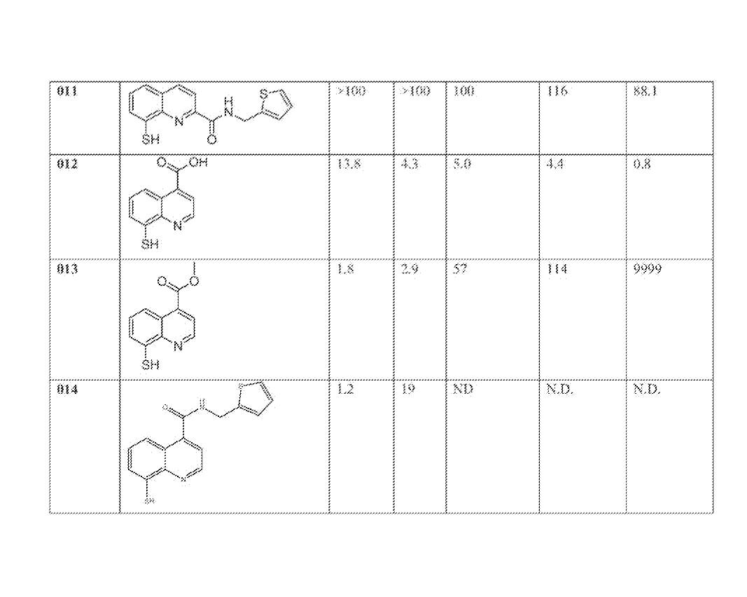

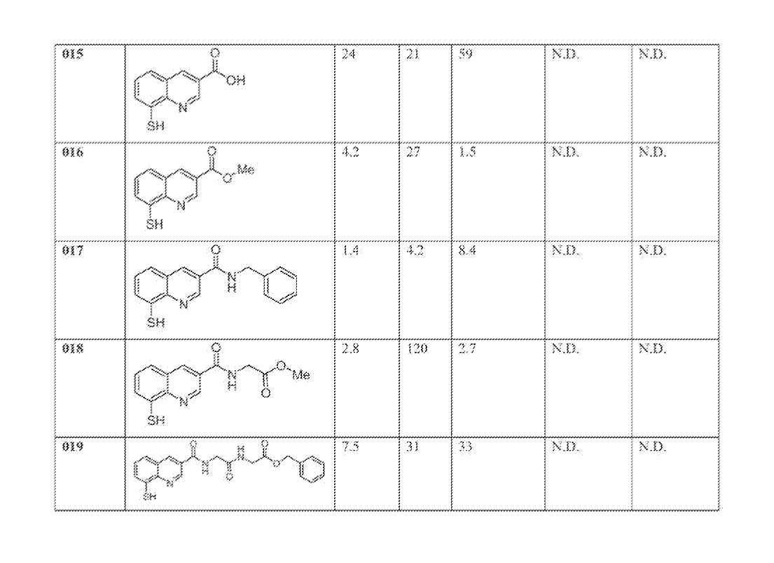

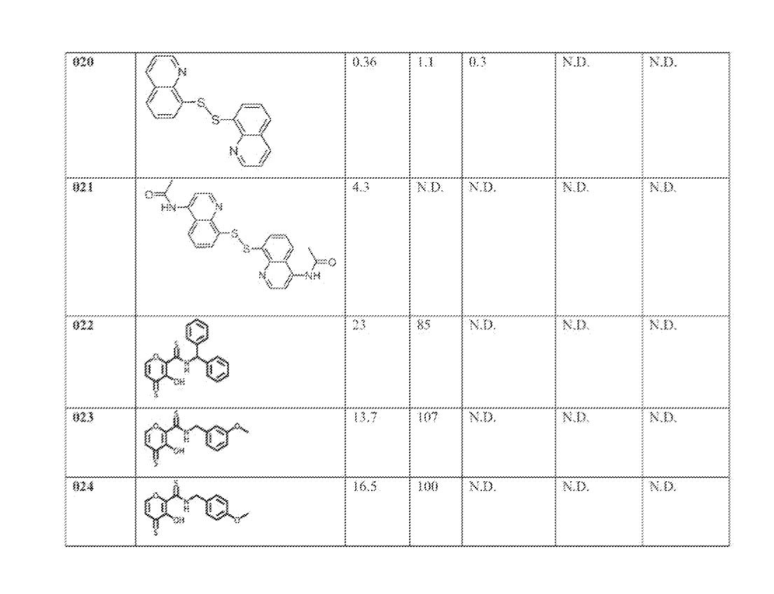

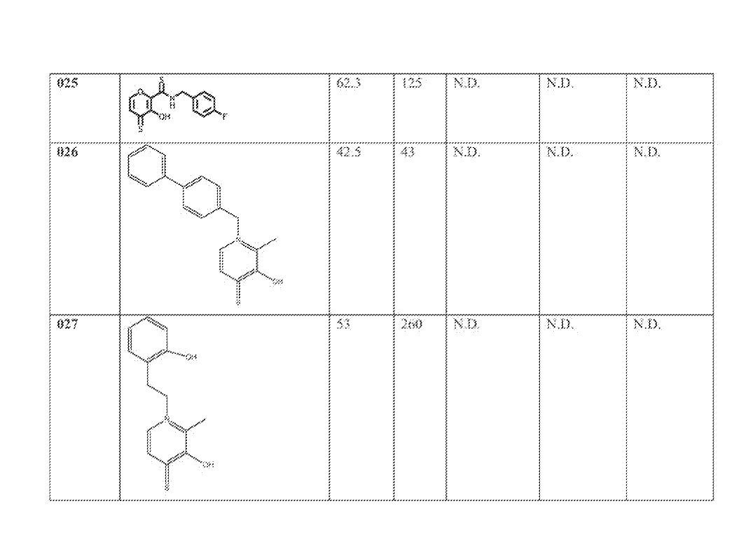

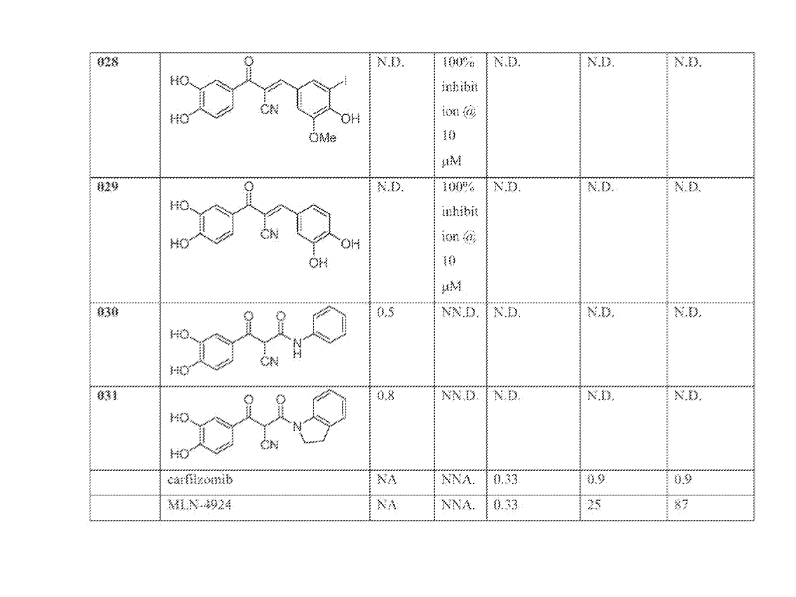

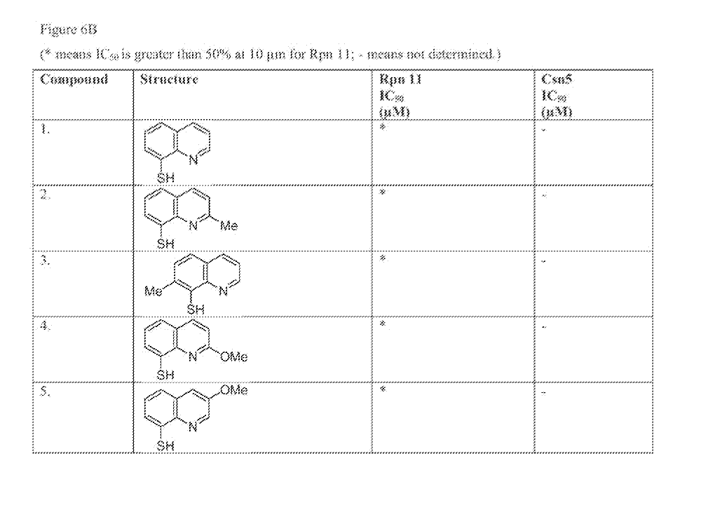

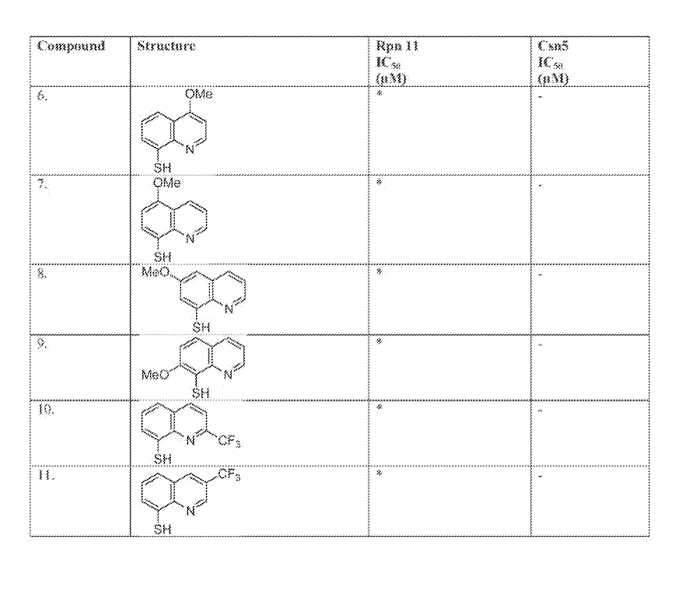

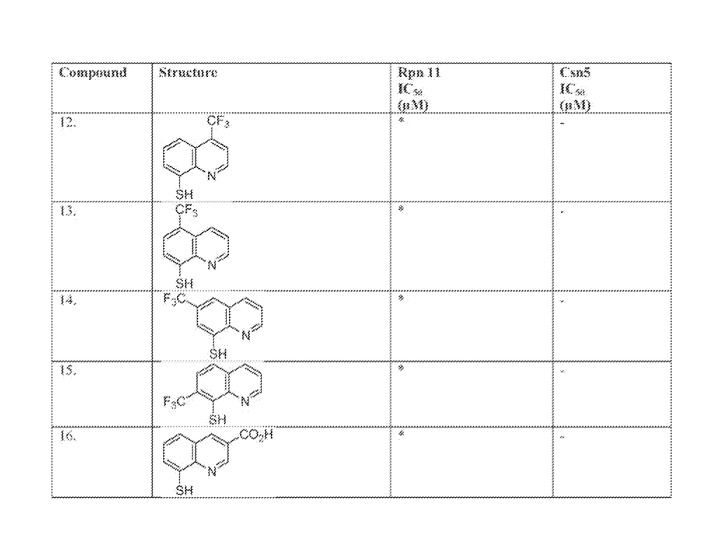

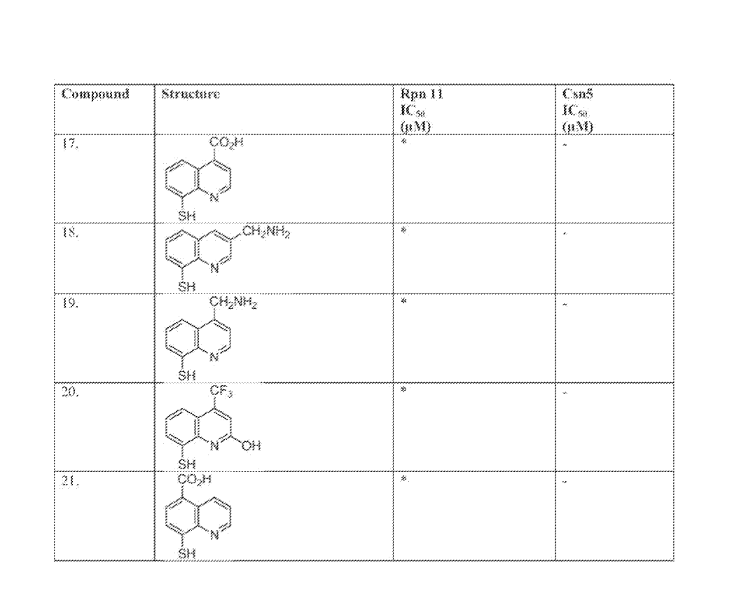

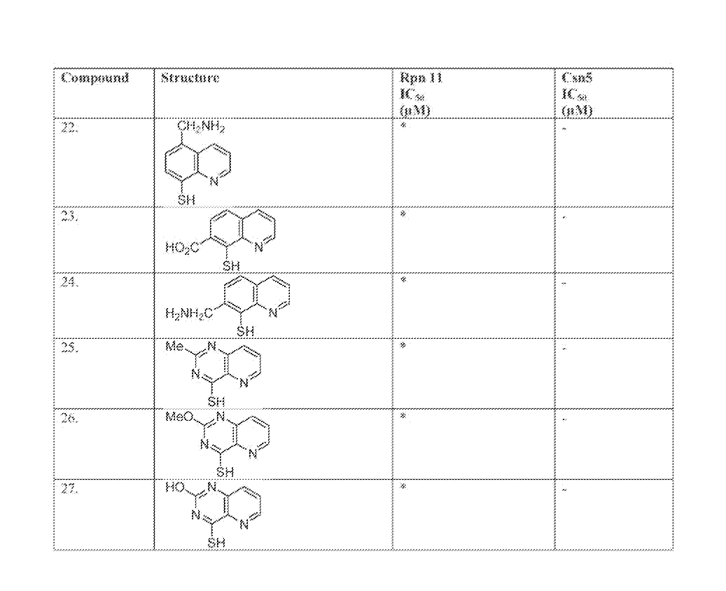

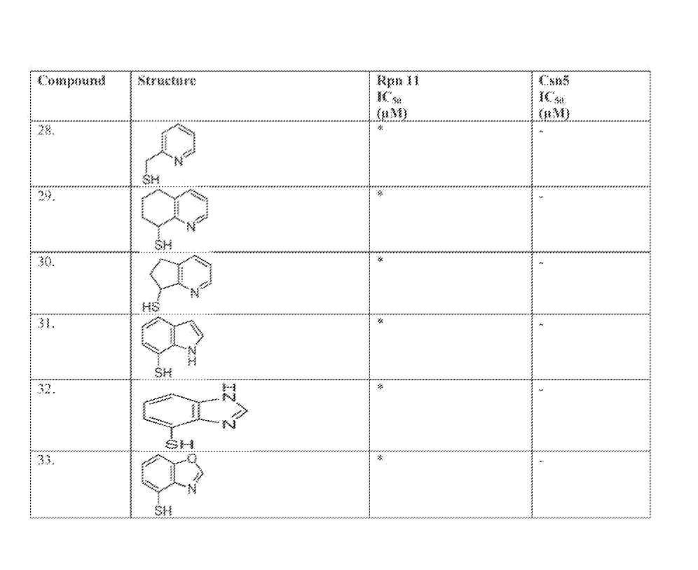

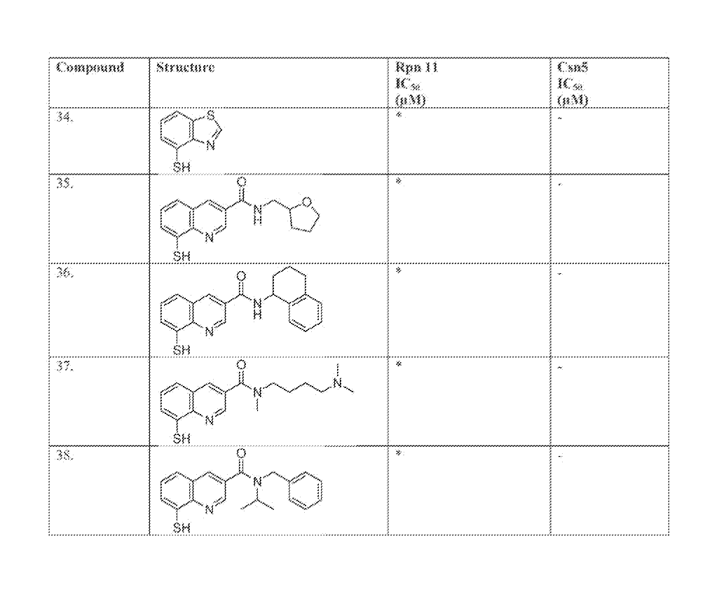

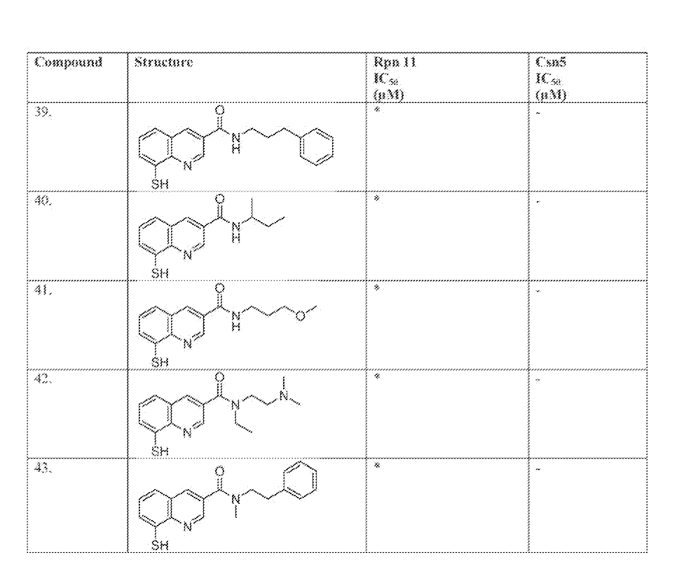

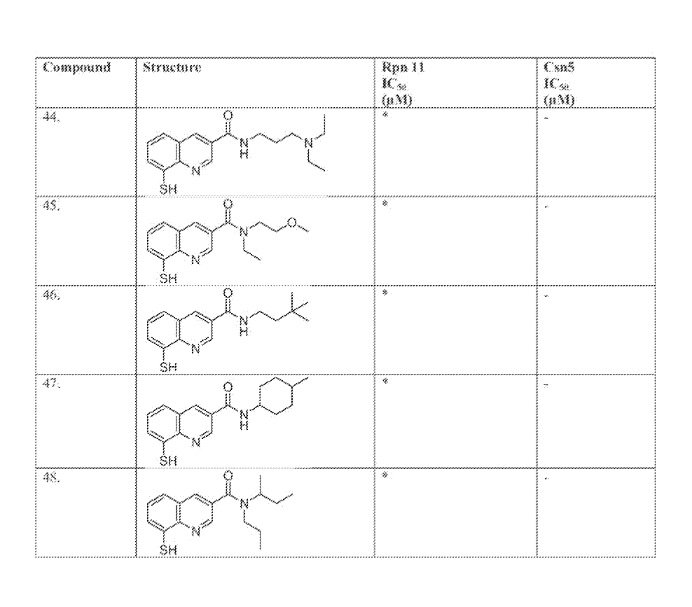

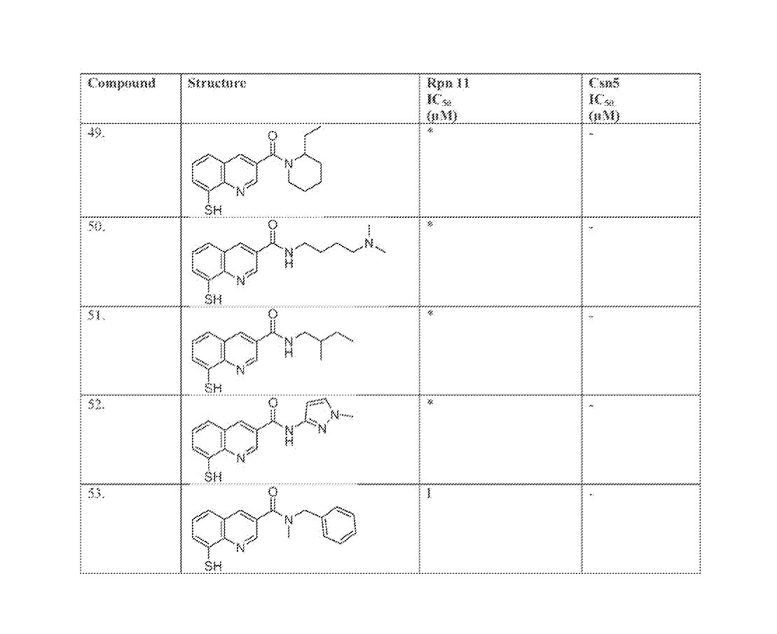

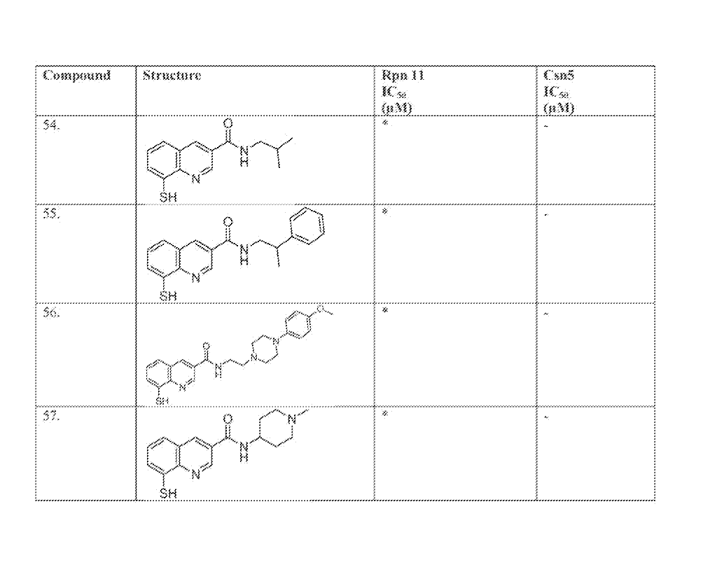

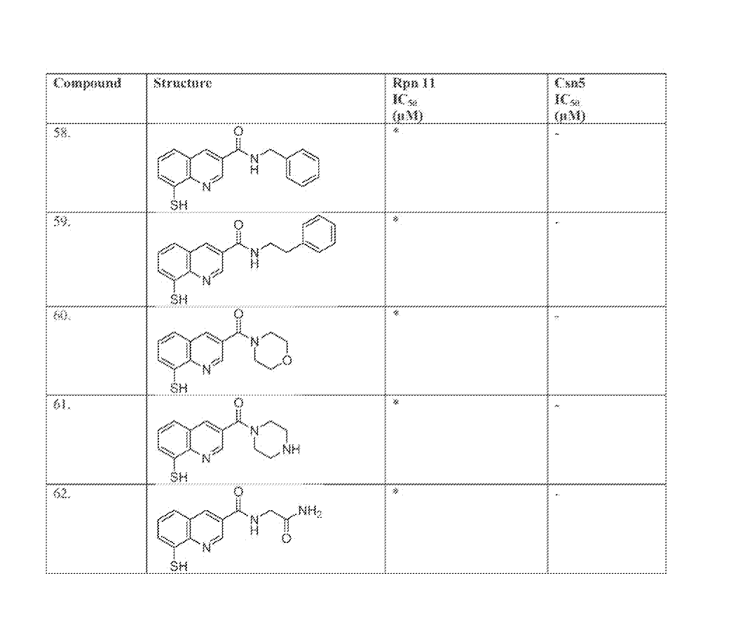

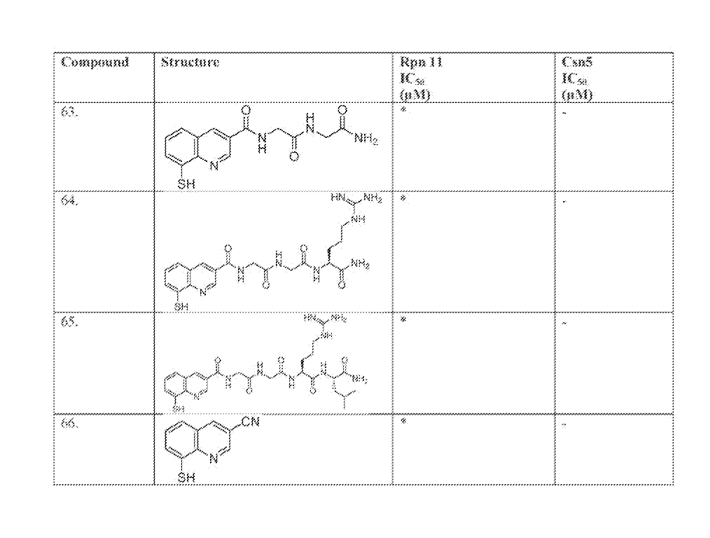

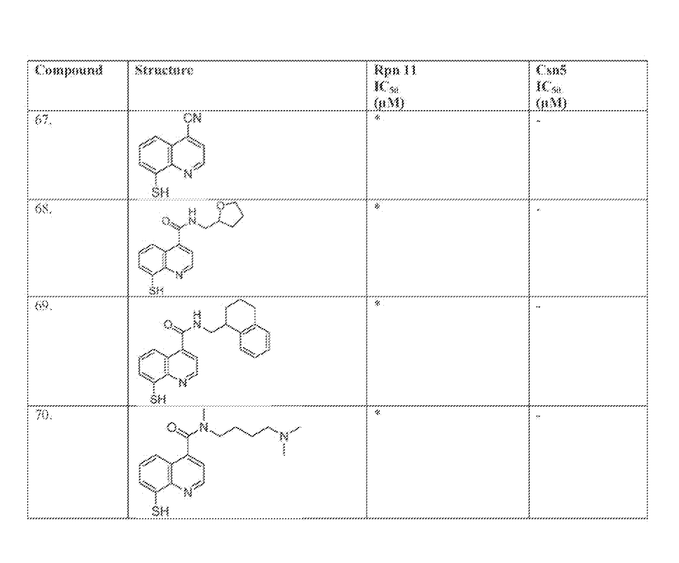

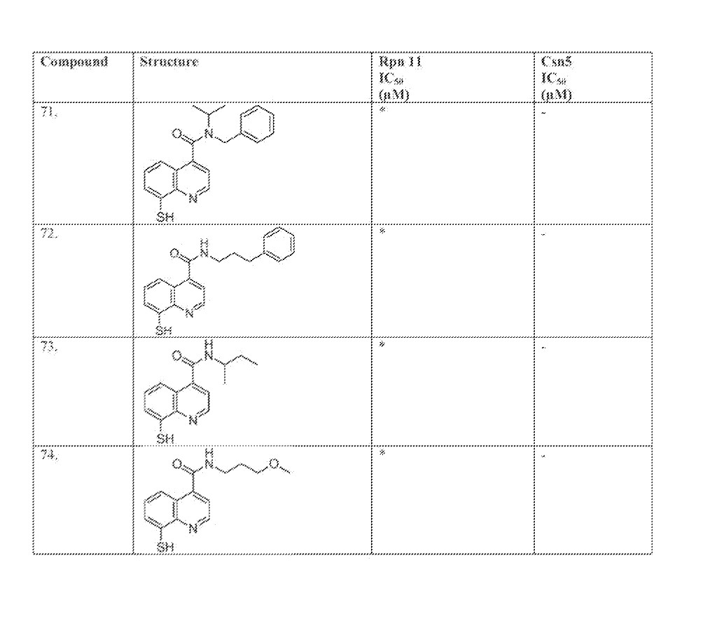

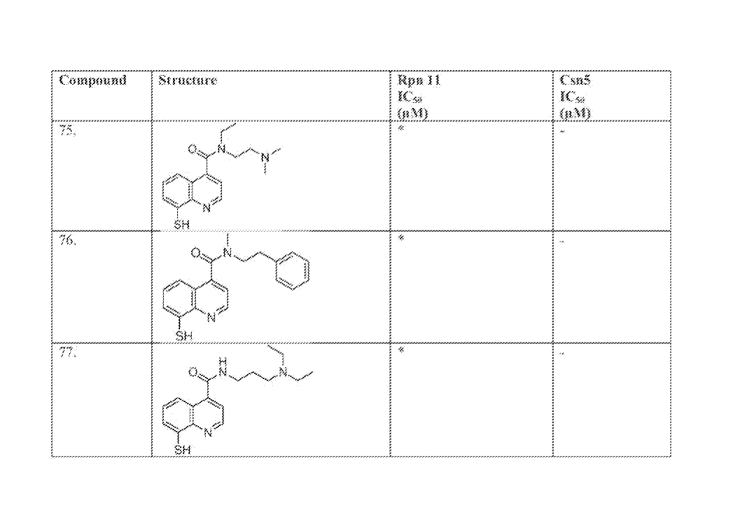

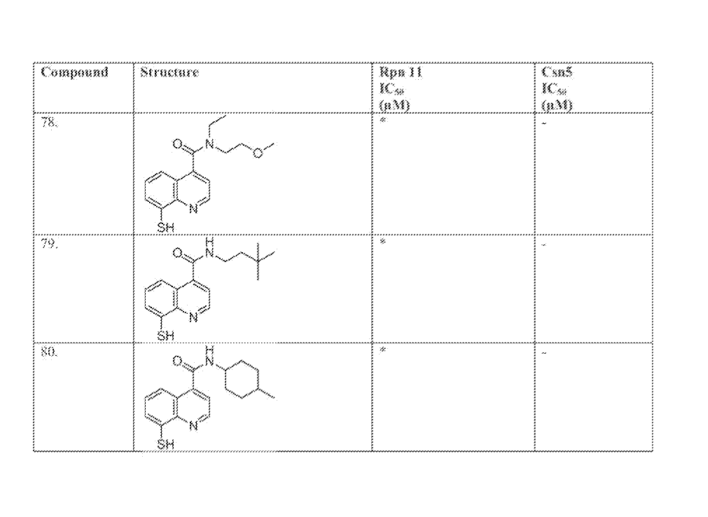

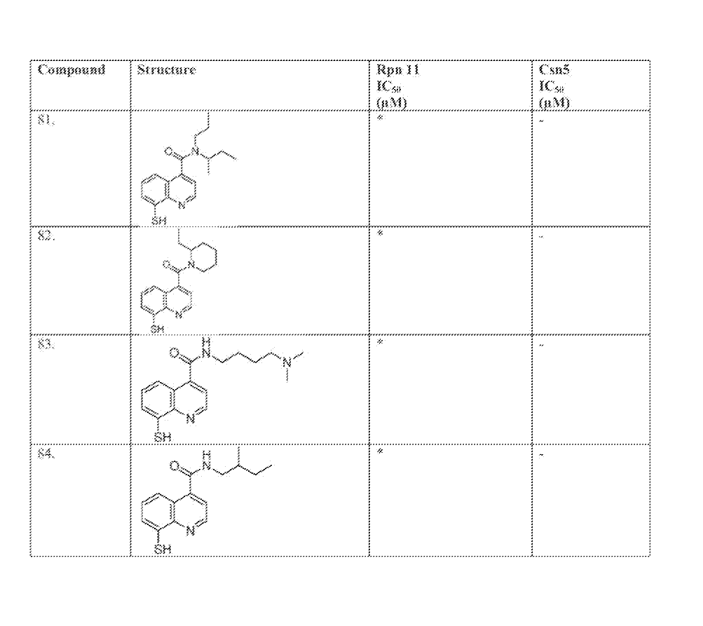

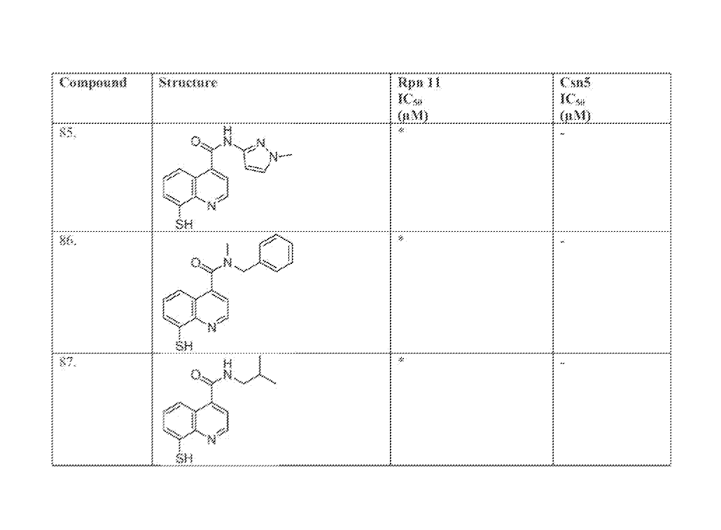

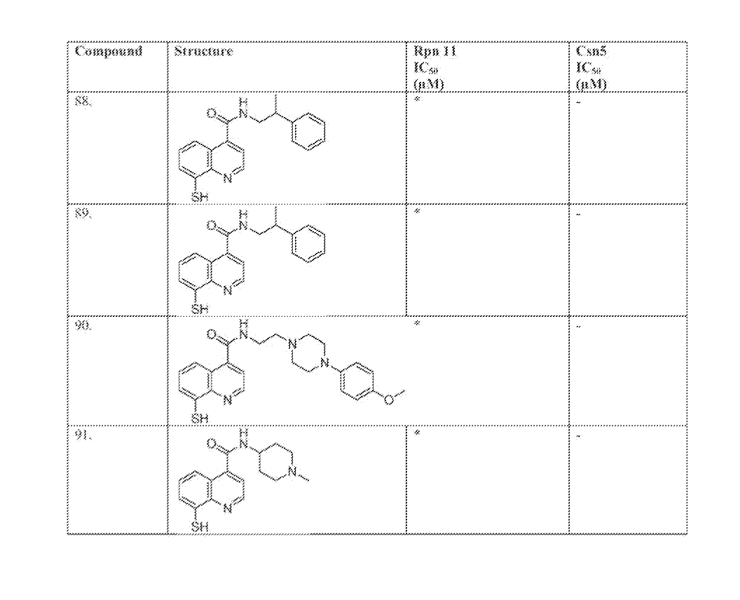

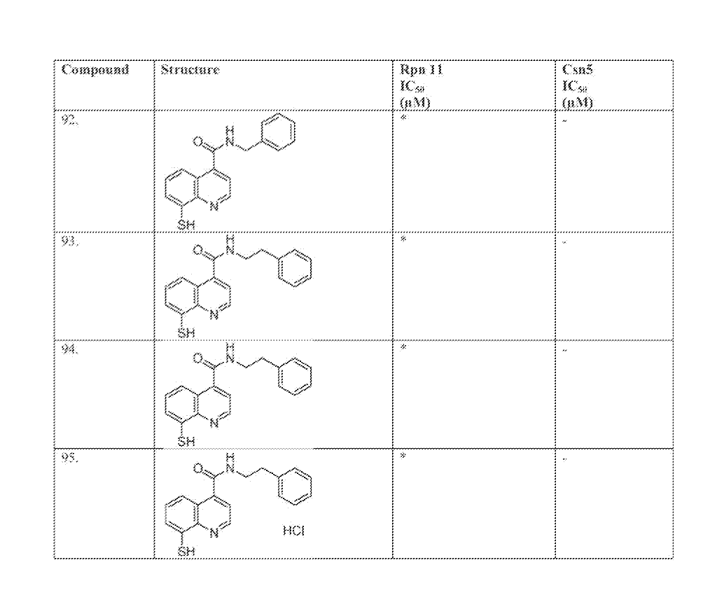

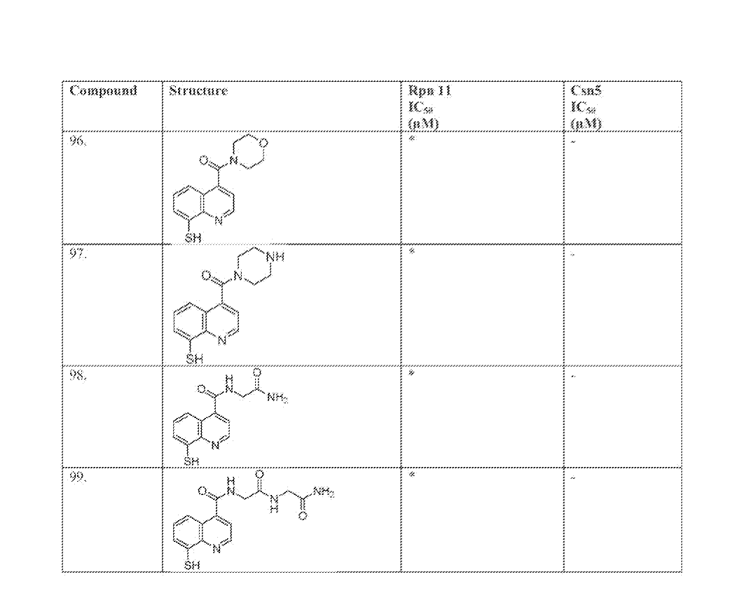

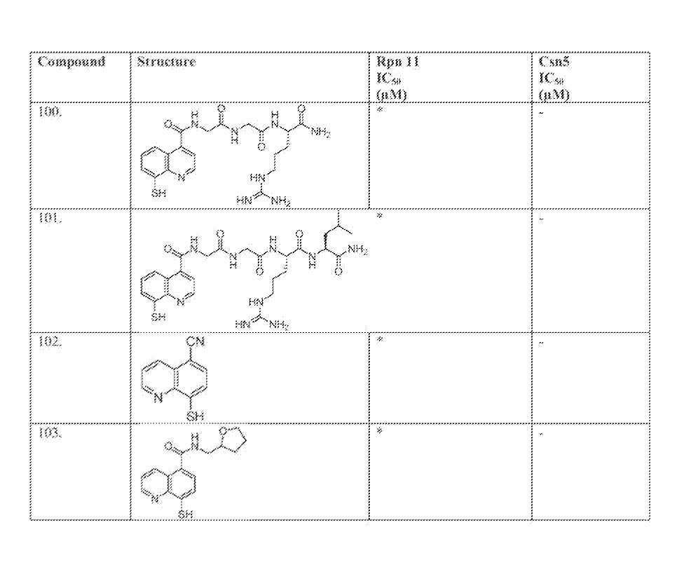

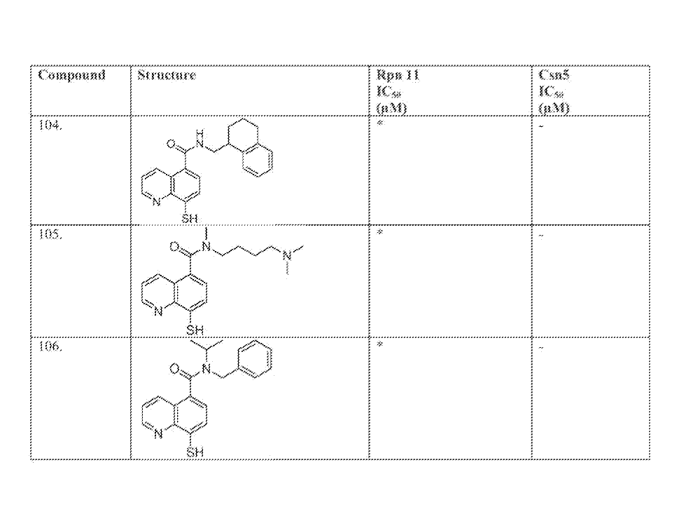

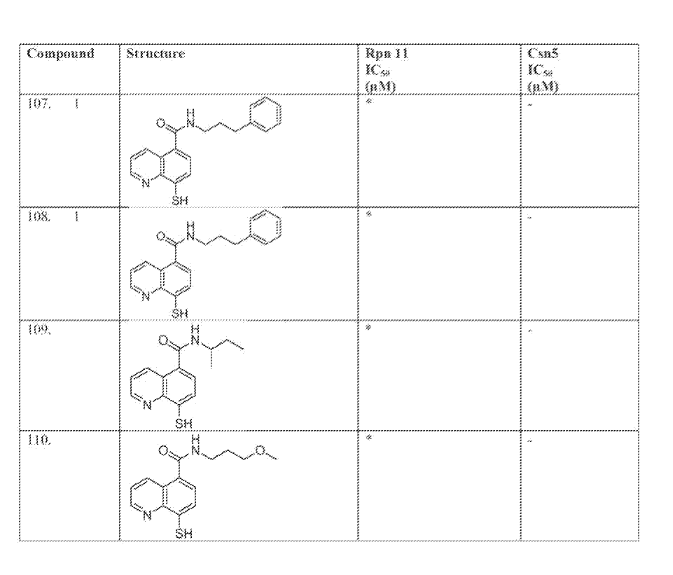

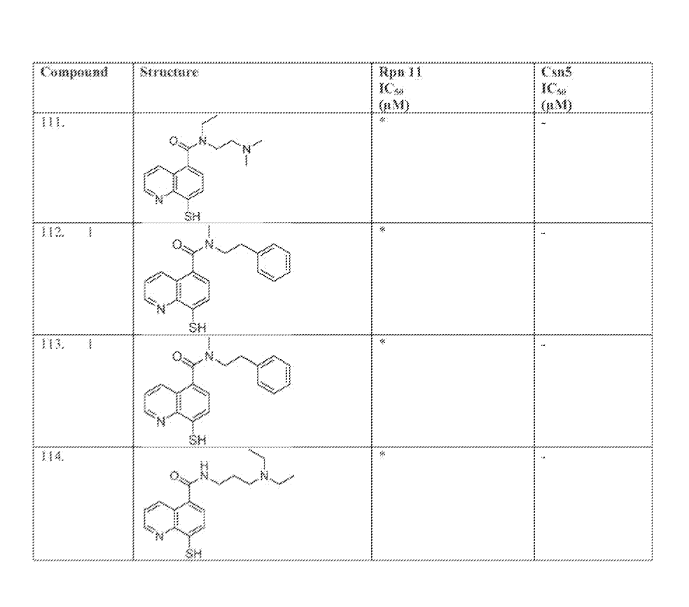

The chemical substituents appended to any of these frameworks a through r may be positioned at any of the above designated locations of the framework as indicated by the foregoing position numbers. Preferably, one to four chemical substituents are appended, preferably, one or two, more preferably one. The chemical substituents used with any of the foregoing frameworks include any of the following substituents as well as any combination thereof. The number designations for the carbons include all integers between the lowest and highest number. Individual numbers of carbon atoms separate and distinct from other numbers of the same group are also included. For example for an alkyl of 1 to 6 carbons, an alkyl group of 1, 2, 3, 4, 5 or 6 carbons is included as well as each individual number designation separate and distinct from other number designations so that the alkyl of 1 to 6 carbon includes separately, methyl, ethyl, propyl, butyl, pentyl and hexyl.
1) Alkyl and branched alkyl of 1 to 6 carbons,
2) Alkoxy and branched alkoxy of 1 to 6 carbons,
3) Amine,
4) Carboxylic acid,
5) Carboxylic ester wherein the alkoxy group of the ester is from 1 to 6 branched or straight carbons or the alcohol esterifying group is phenoxy,
6) Branched or straight alkylenyl carboxylic acid or ester of 2 to 7 carbons in the alkylenyl group and 1 to 6 branched or straight carbons in the ester group,
7) Branched or straight alkylenyl amine of 1 to 6 carbons,
8) Branched or straight perfluoroalkyl of 1 to 6 carbons,
9) Branched or straight trifluoroalkyl of 1 to 6 carbons wherein the trifluoro group is on the terminating or end carbon,
10) Hydroxyl,
11) Branched or straight alkylenyl hydroxyl of 1 to 6 carbons,
12) Carboxamide eg., —CONH₂
13) Aminocarbonylalkyl, eg., —NHCOR, wherein R is alkyl of 1 to 6 carbons,
14) Branched or straight alkylenylcarboxyamide of 1 to 6 carbons, e.g., —RCONH₂,
15) alkyleneaminocarbonylalkyl, eg., —RNHCOR, wherein the alkylenyl is branched or straight and is 1 to 6 carbons and the alkyl is branched or straight and is 1 to 6 carbons,
16) N-substituted carboxamide, wherein the N substituent is an aryl group, heteroaryl group heterocycle group as defined in the DEFINITIONS section, eg., —CONHAr or —CONHHet,
17) N-substituted carboxamide wherein the N substituent is an alkaryl group, a alkyheteroaryl group or a alkheterocycle group as defined in the DEFINITIONS section, and wherein the “alk” group is an alkylenyl or branched alkylenyl group of 1 to 6 carbons, eg., —CONH—R—Ar or —CONH—R-Het,
18) N-substituted carboxamide wherein the N substituent is a branched or straight alkyl group of 1 to 10 carbons, the polyfluorinated version thereof, or a substituted version thereof eg., —CONH—R, wherein the substituent of the alkyl group is halogen, cyano, carboxyl, ester of 1 to 6 branched or straight chain carbons in the alkoxy or phenoxy portion, carboxamide, sulfonamide, alkoxy of 1 to 6 carbons, urea, carbamate of 1 to 10 carbons, amine, mono or dialkyl amine having from 1 to 6 carbons in the alkyl group with the alkyl group being straight or branched, hydroxyalkyl of 1 to 10 branched or straight chain carbons or a cycloalkyl group as defined in the DEFINITIONS section,
19) Preferred aryl, heteroaryl and heterocycle groups for 16 and 17 include phenyl, halogen substituted, phenyl, aminophenyl, benzoic acid, tolyl, xylyl, anisolyl, trifluoromethylphenyl, benzyl, tetrahydrofuran, pyrrolidinyl, tetrahydronaphthalene, cyclohexyl or alkyl substituted cyclohexyl with the alkyl group having 1 to 6 carbons, cyclohexyl or alkyl substituted cyclohexyl with the alkyl group having 1 to 6 carbons, cyclopentyl or alkyl substituted cyclopentyl with the alkyl group having 1 to 6 carbons, pyrazolyl, imidazolyl, piperidinyl, piperazinyl, pyrimidinyl, morpholinyl, pyrrolyl, thiophenyl, substituted versions of any of the foregoing aryl, heteroaryl or heterocycle groups wherein the chemical substituent is halogen, cyano, carboxyl, ester of 1 to 10 branched or straight, chain carbons in the alkoxy or phenoxy portion, amine, carboxamide, sulfoxamide, urea, carbamate of 1 to 10 carbons, hydroxyl, thiol, alkoxy, anisolyl, phenyl, benzyl or a cycloalkyl group as defined in the DEFINITIONS section.
20) Derivatives of 16, 17 and 18 wherein the N of the carboxamide has a second substituent and the second substituent is a branched or straight chain alkyl of 1 to 6 carbons,
21) N-substituted carboxyamide wherein the N substituent is a mono, di, tri or tetra amino acid and the amino acid moieties include glycinyl, alaninyl, leucinyl, valinyl, phenylalaninyl, lysinyl, argininyl, histidinyl, serinyl, aspariginyl, glutaminyl, aspartic, glutamic such that the amino acid moieties may be combined in any combination of two, three or four moieities including but not limited to a tetramer of four different moieties, a tetramer of two and two different moieties, a tetramer of three of one moiety and one of a different moiety, a trimer of two of one moiety and one of another moiety or a trimer of three different moieties, a dimer of two different moieties of of the same moiety, and a monomer of any of the designated moieties. The nitrogen of an amino acid moiety may serve as the nitrogen of the carboxyamide group. The C-terminus of the amino acid monomer, dimer or trimer may be a carboxylic acid or a carboxamide. The order of amino acid moieties in the tetramer, trimer or dimer may be any order.
22) In addition to the groups of substituents set forth in 1 through 21 above, each individual substituent and individual combination is included separately and individually as if it were individually recited.
23) Additional embodiments of the compounds of the invention further include each individual compound listed on FIGS. 5 and 6a and 6b.

### Synthetic Methods for Preparation of the Compounds of the Invention

Compounds disclosed herein may be made by a variety of methods, including methods known in the art of organic synthesis. For example, the compounds disclosed herein can be made according to Org. Biomol. Chem. 2003, 1, 4248-4253, incorporated herein by reference for its teachings with respect to organic synthesis. It is well understood that protecting groups for sensitive or reactive groups are employed where necessary in accordance with general principles of chemistry. Protecting groups are manipulated according to standard methods of organic synthesis (see, for example, T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of the invention.

Those skilled in the art will recognize if a stereocenter exists in compounds of the invention. Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be performed by any suitable method known in the art. See, for example, Stereochemistry of Organic Compounds by E. L. Eliel, S. H. Wilen, and L. N. Mander (Wiley-Interscience, 1994).

The following methods illustrate techniques and procedures for forming the aromatic, aliphatic or aliaromatic frameworks and linkage sites for side chain moiety addition according to the invention. Although not every aromatic, aliphatic or aliaromatic framework with every X and Y variation described on pages 5, 6, 42-45 are illustrated, the skilled practitioner will readily be able to adopt the following methods to frameworks not shown. Those adaptations are routine and ordinary in the art. Examples of side chain moiety linkage with frameworks are provided in the Examples section. While not all possible linkage reactions are illustrated, the skilled practitioner will readily be able to utilize these illustrations and reaction schemes provided in the synthetic treatises cited below to prepare each and every variation of framework moiety linked to side chain moieties according to the invention.

While several of the illustrated aromatic, aliphatic and aliaromatic frameworks are known, incorporation of side chain moieties into these frameworks produce new, bioactive compounds. According to the invention, the introduction of the side chain moieties at least in part will be of benefit to the bioactivity and selectivity of the resulting compound relative to the bioactivity and selectivity of the framework without a side chain moiety. Side chain moieties can be introduced by formation of amide, ester, ether, substituted amine, substituted thioether, imine (Schiff base), carbon-carbon attachment, and peptide and pseudopeptide linkages. By way of exemplary information, amides can be formed from carboxylic acids and amines by activated ester coupling, acyl halide or azide coupling, activated anhydride coupling, carboxylic acid-amine coupling using a coupling agent such as carbodiimide, carbonyl diimidazole, pyridinium salts and other coupling agents disclosed in Advanced Organic Chemistry, (March) page 420, cited infra and any number of additional amide formation techniques reported by March. Esters can be formed in similar ways through use of alcohols with activated esters, activated anhydrides or acyl halides and diazides. Substituted amines can be formed by amine substitution on saturated carbons by displacement of facile leaving groups. Ethers can be formed by diazo reactions with alcohols. Substituted thioethers can be formed similarly by reaction with thioalcohols. Imines can be formed by reaction of primary amines with ketones or aldehydes. Carbon-carbon linkage can be formed by Grignard reaction with an aldehyde or ketone followed by reduction of the alcohol by tosylate formation and hydrogenation. Alternatively, a phosphorylid can be reacted with the Grignard reagent followed by reduction of the resulting olefin. Peptides can be formed by reaction of an activated ester group of a peptide with an amine group. In all substituent additions, either reactant may be the linking substituent on the framework moiety, the other reactant being the coupling agent on the side chain moiety. Protecting groups to prevent spurious reaction of other substituents on the framework moiety and/or the side chain moiety may also be employed.

A linkage study can begin with a carboxamide linkage with the carbonyl moiety attached to the framework. If the substituent provides acceptable bioactivity, the linker can be reversed to attach the nitrogen moiety to the framework. Placing a methylene group between the framework and the carboxamide group can be a third variation for study. Conversion of the linker to an amine-group, an ether group, a thioether group or a carbon-carbon group will allow examination of the bioactivity of the subject side chain moiety linked to the framework with all of these linkers.

General and specific methods for attachment of side chain moieties to the aromatic, aliphatic and aliaromatic frameworks through these linking groups follow the discourse for formation of such linking groups set forth in Advanced Organic Chemistry, 4^{th}-6^{th}Ed., Jerry March, John Wiley, New York, 1992, 2007; and in Modern Synthetic Reactions, 1^{st} and 2^{nd} Ed., H. O. House, W. A. Benjamin, New York, 1965 and 1972. In this context, linking groups are the same as functional groups such as amide, ester, amine, ether, thioether, imine and peptidyl groups. Protecting groups and their manipulation for protection in the course of specific synthetic reactions are disclosed in Protective Groups in Organic Synthesis, 3^{rd} Ed., T. W. Green and P. Wuts, Wiley & Sons Pub., New York, 1999), the disclosure of which is incorporated herein by reference. The disclosures of these references are incorporated herein by reference, and in particular are incorporated for illustration of synthetic methods for synthesis of the aromatic, aliphatic and aliaromatic frameworks with linking moieties, for illustration of synthetic methods for combining side chain moieties to those linking moieties, for illustration of synthetic methods for preparation of such side chain moieties, and for illustration of technical procedures for accomplishing such synthesis.

### METHODS

Method 1. Conversion of carboxylic-substituted-8-F-quinolines into the corresponding substituted-8-SH-quinolines:

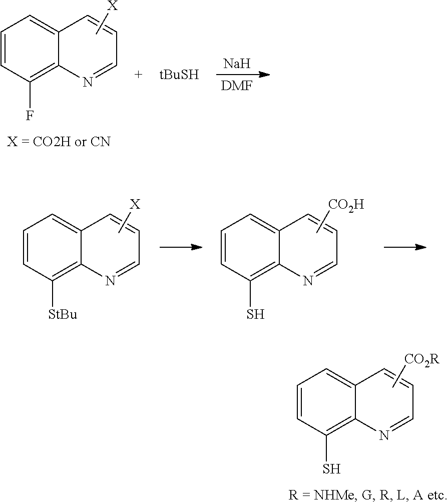

All amino acids or peptides may optionally be linked via its N-terminus. See Organic & Biomolecular Chemistry, 1(23), 4248-4253; 2003, incorporated herein by reference.

Method 2. Conversion of amino-substituted-8-F-quinolines into the corresponding substituted-8-SH-quinolines:

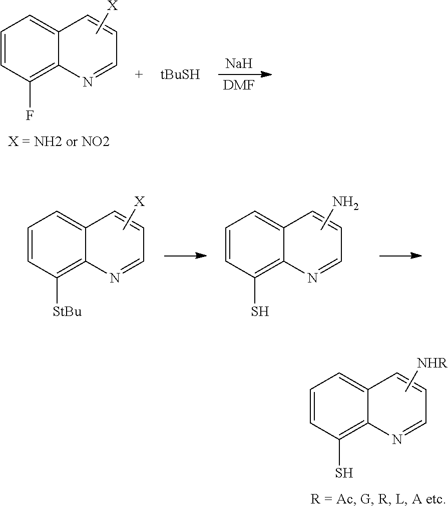

All amino acids or peptides may optionally be linked via C-terminal. See Organic & Biomolecular Chemistry, 1 (23), 4248-4253; 2003, incorporated herein by reference.

Method 3. Conversion of carboxylic-substituted-8-OH-qninolines into the corresponding substituted-8-SH-quinolines:

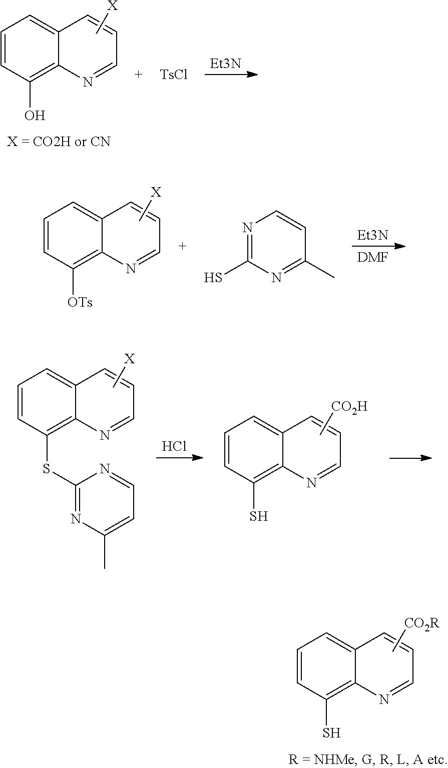

All amino acids or peptides will be linked via its N-terminus. See Journal of Heterocyclic Chemistry, 28 (3), 657-65; 1991, incorporated herein by reference.

Method 4. Conversion of amino-substituted-8-OH-quinolines into the corresponding substituted-8-SH-quinolines:

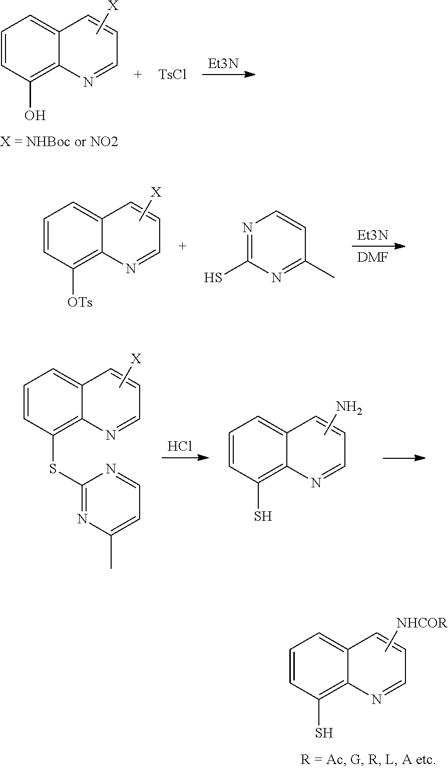

All amino acids or peptides may optionally be linked via C-terminus. See Journal of Heterocyclic Chemistry, 28 (3), 657-65; 1991, incorporated herein by reference.

Method 5. Conversion of substituted-8-NH₂ (or NO₂)-quinolines into substituted-8-SH-quinolines:

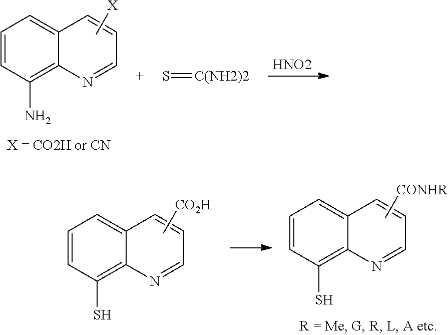

All amino acids or peptides will be linked via N-terminus. See Latvijas PSR Zinatnu Akademijas Vestis, Kimijas Serija, (6), 659-62: 1988; Journal of the Chemical Society, Perkin Transactions 1: Organic and Bio-Organic Chemistry (1972-1999), (3), 671-2; 1982, incorporated herein by reference.

Method 6. To synthesize CH₂NH₂-ubstituted-8-thiol-quinolines and their derivatives:

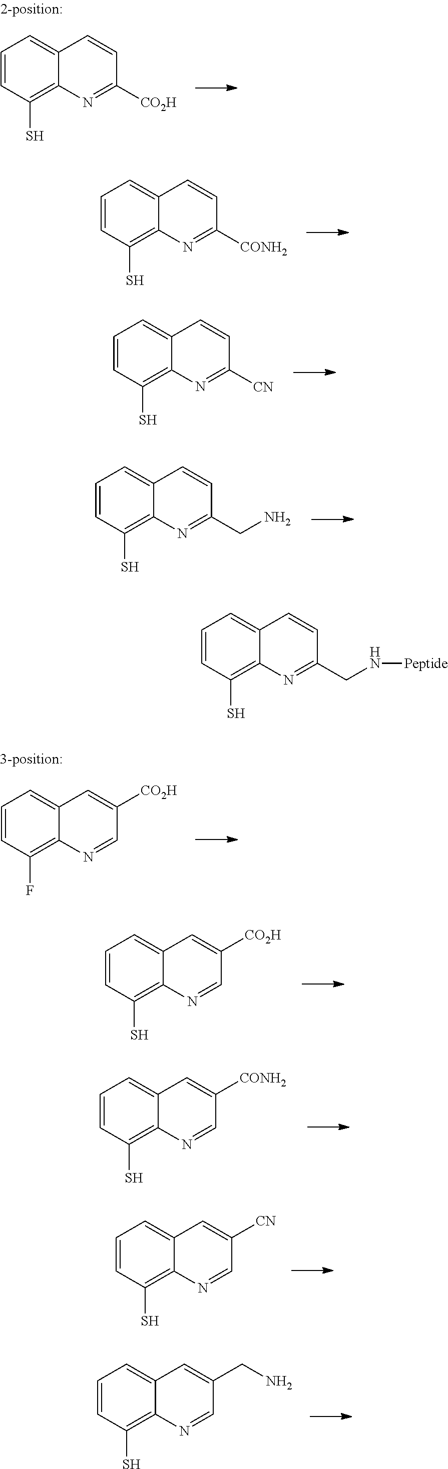

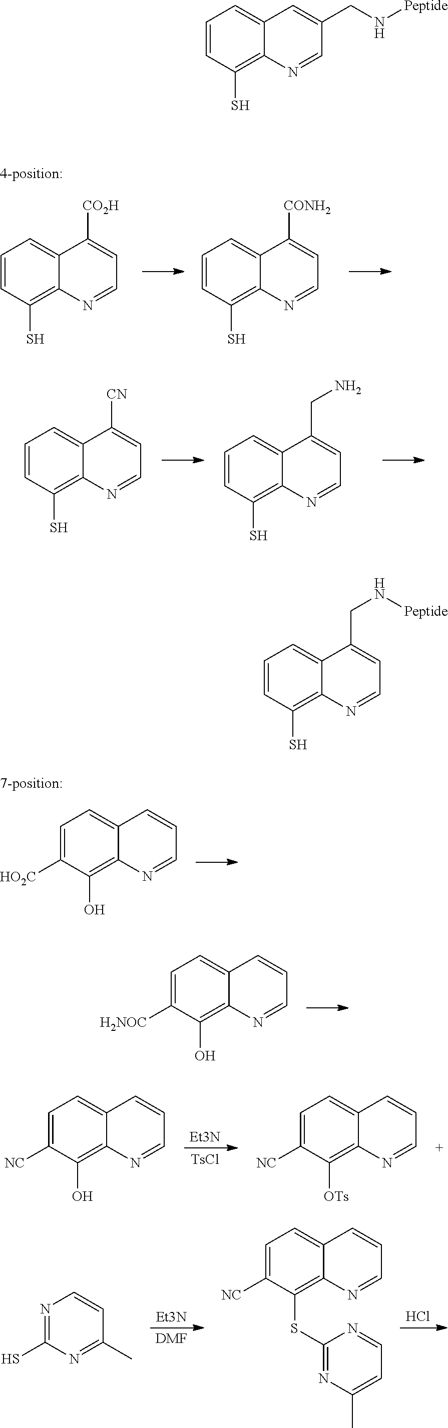

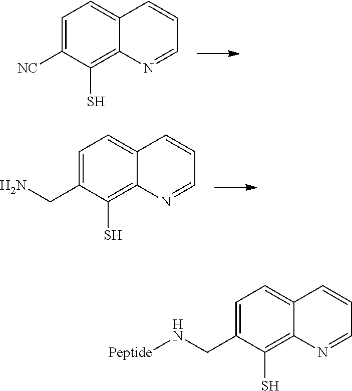

See Organic & Biomolecular Chemistry, 1 (23), 4248-4253, 2003.

Method 7. To synthesize amino-substituted-8-thiol-quinolines and their derivatives:

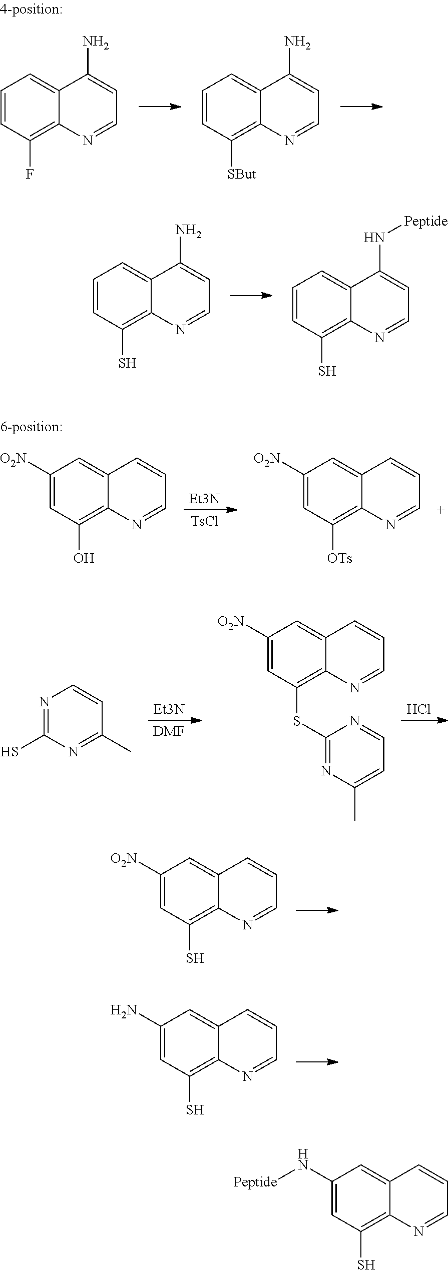

Method 8:

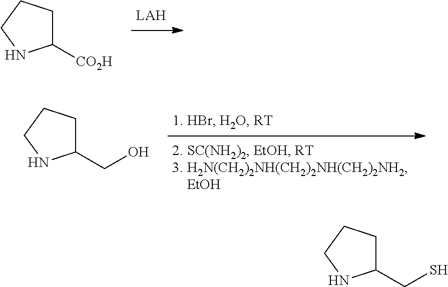 [SMILES: O=C(O)C1CCCN1 ; OCC1CCCN1 ; SCC1CCCN1]

Method 9:

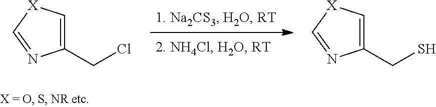

Method 10:

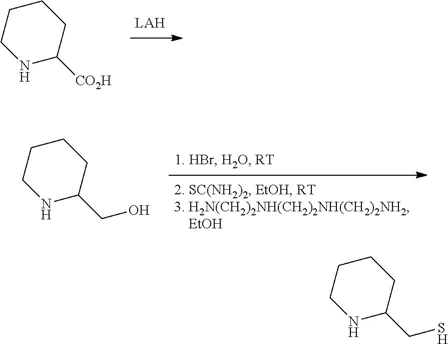 [SMILES: O=C(O)C1CCCCN1 ; OCC1CCCCN1 ; SCC1CCCCN1]

Method 11:

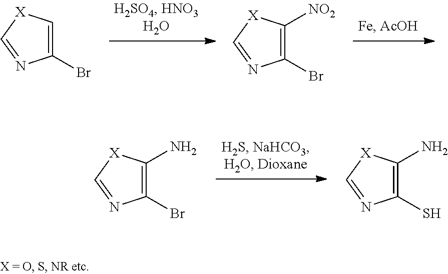

Method 12:

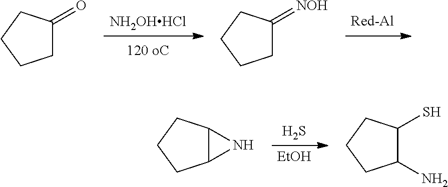

Method 13:

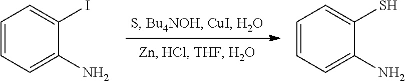 [SMILES: Nc1ccccc1I ; Nc1ccccc1S]

Method 14:

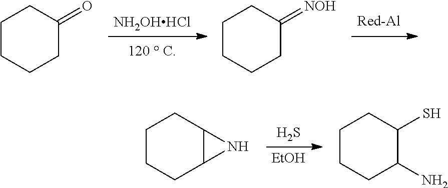

Method 15:

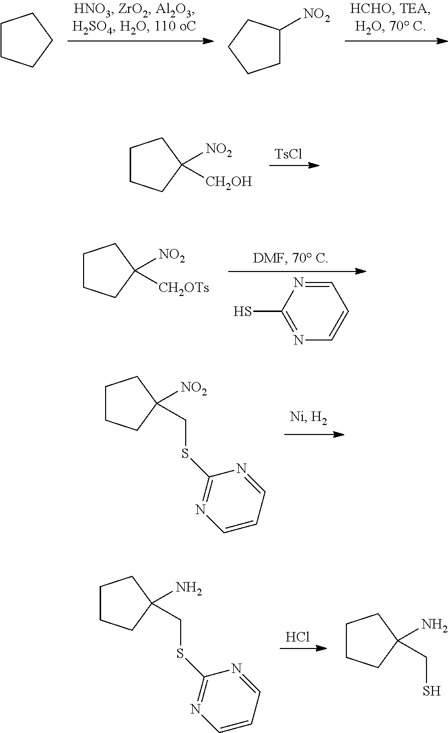

Method 16:

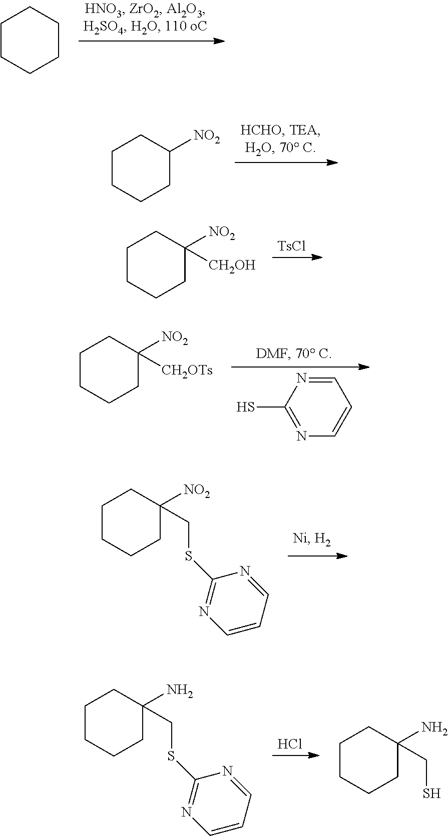

Method 17:

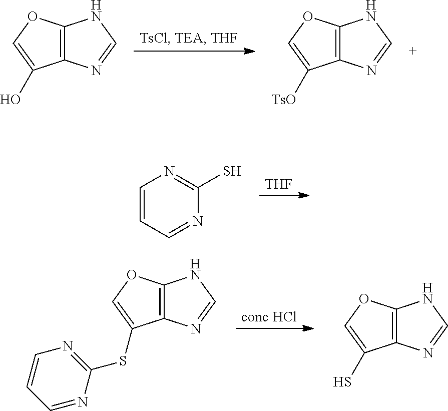

Method 18:

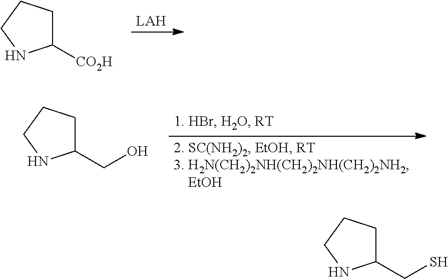

Method 19:

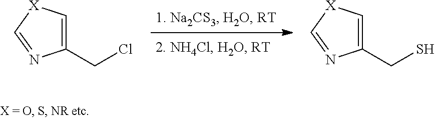

Method 20:

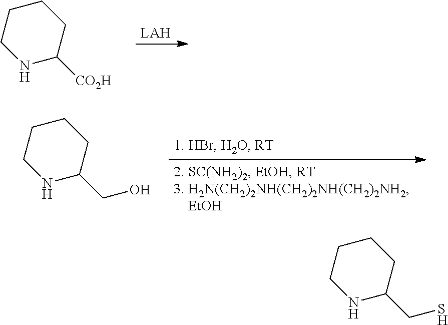

Method 21:

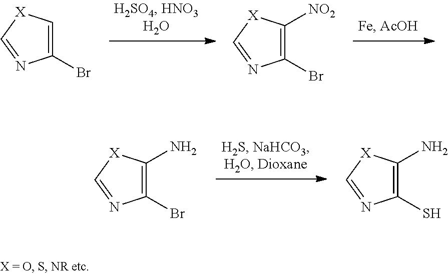

Method 22:

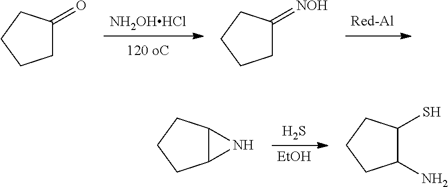

Method 23:

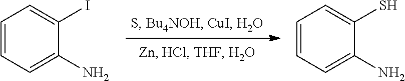 [SMILES: Nc1ccccc1I ; Nc1ccccc1S]

Method 24:

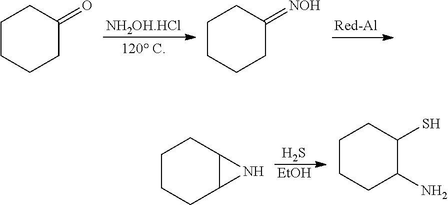

Method 25:

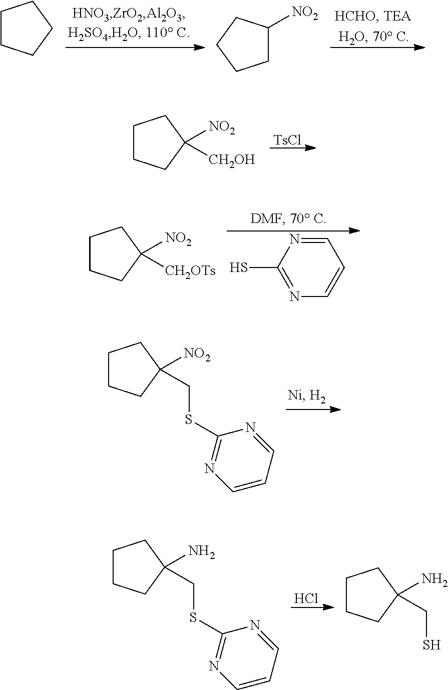

Method 26:

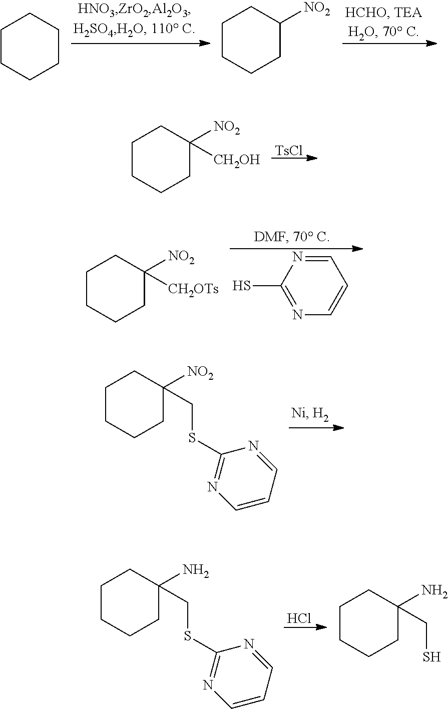

Method 27:

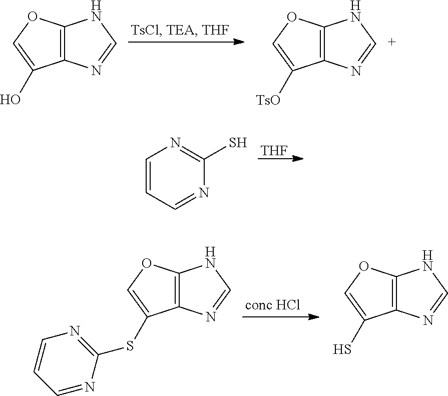 [SMILES: Oc1coc2[nH]cnc12 ; Cc1ccc(S(=O)(=O)Oc2coc3c2N=[C+]N3)cc1 ; Sc1ncccn1 ; c1cnc(Sc2coc3[nH]cnc23)nc1 ; Sc1coc2[nH]cnc12 ; "THF"]

Method 28:

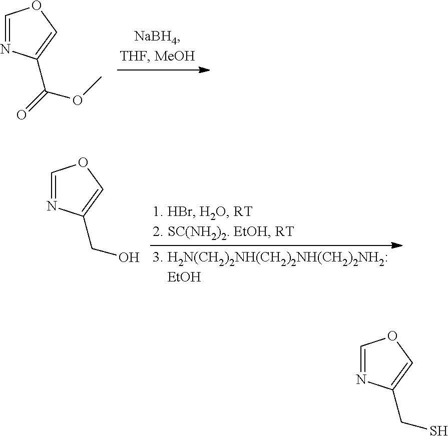

Method 29:

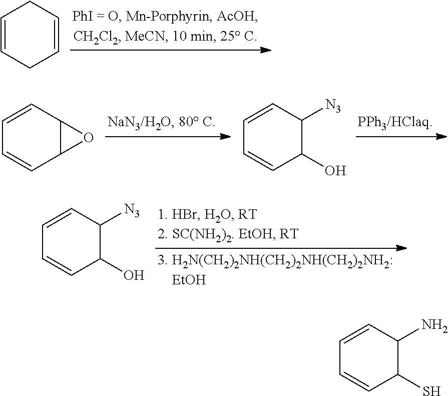

Method 30:

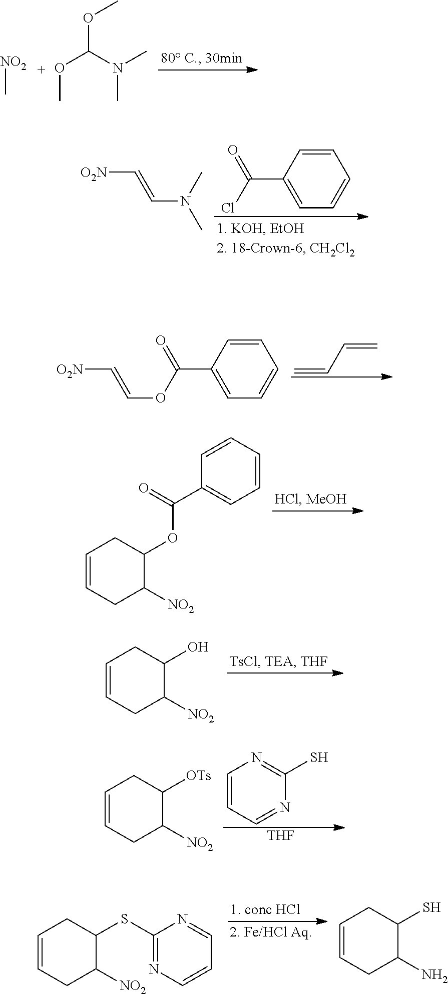

Method 31:

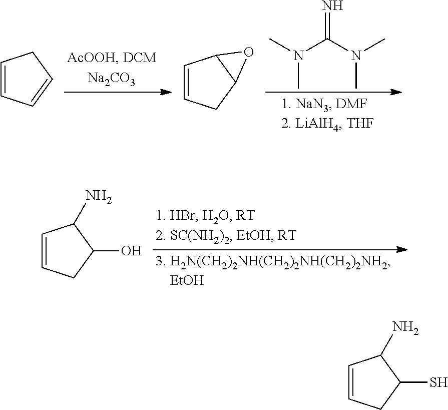

Method 32:

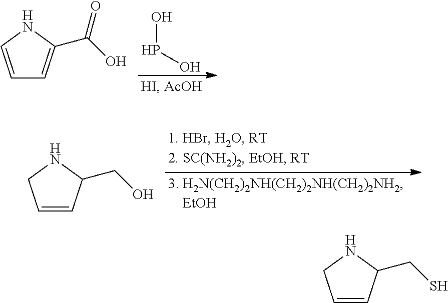

Method 33:

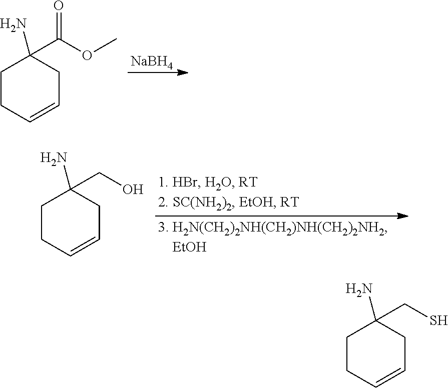

Method 34:

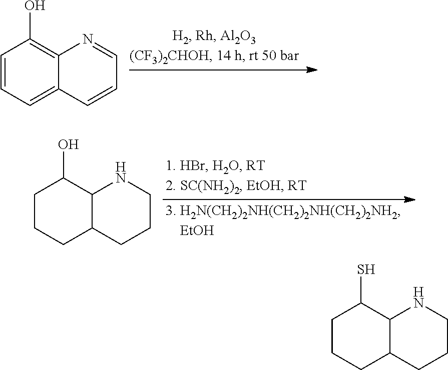

Method 35:

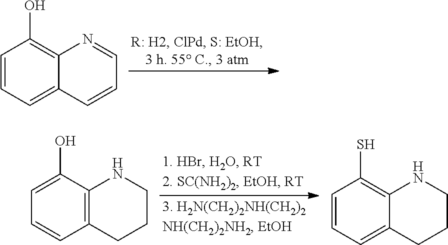

Method 36:

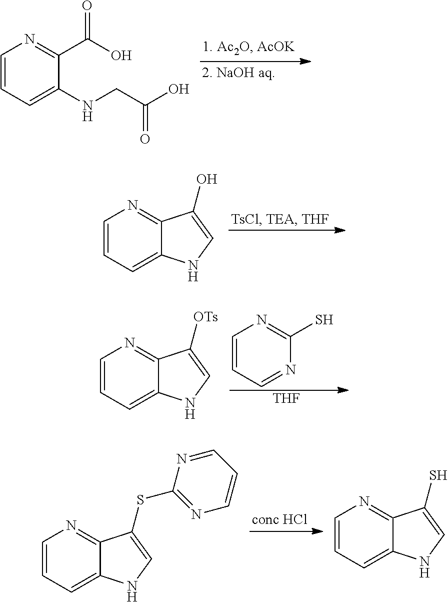

Method 37:

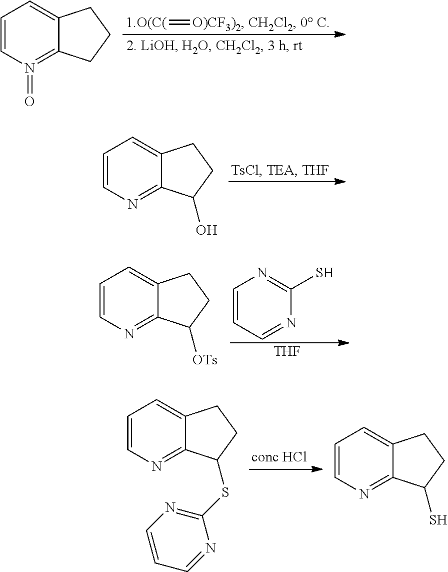

Method 38:

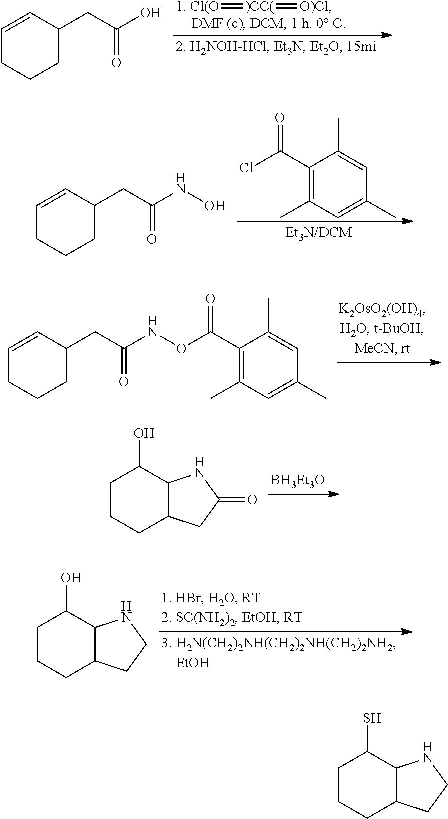

Method 39:

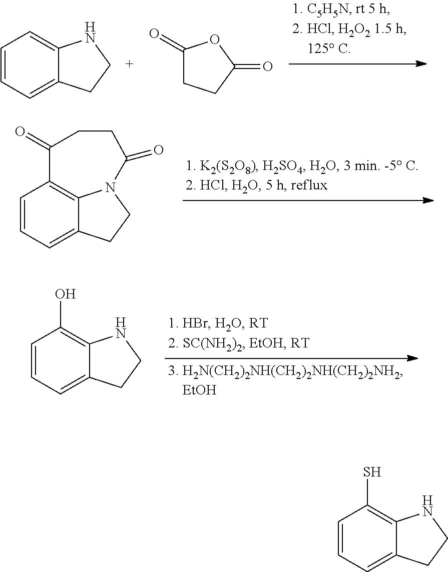

Method 40:

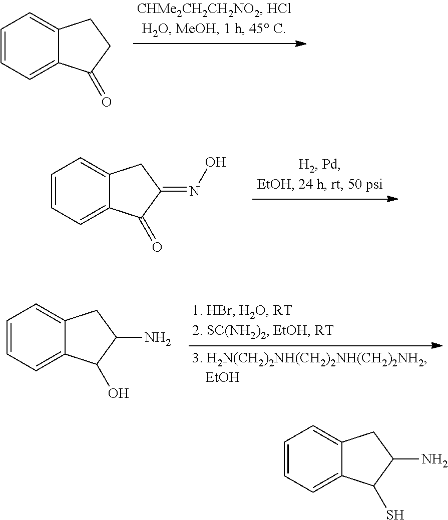 [SMILES: O=C1CCc2ccccc21 ; O=C1/C(=N/O)Cc2ccccc21 ; NC1Cc2ccccc2C1O ; NC1Cc2ccccc2C1S]

Method 41:

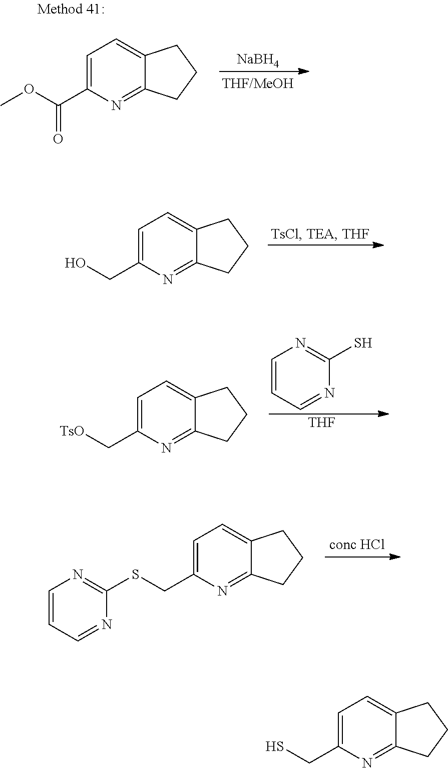

Method 42:

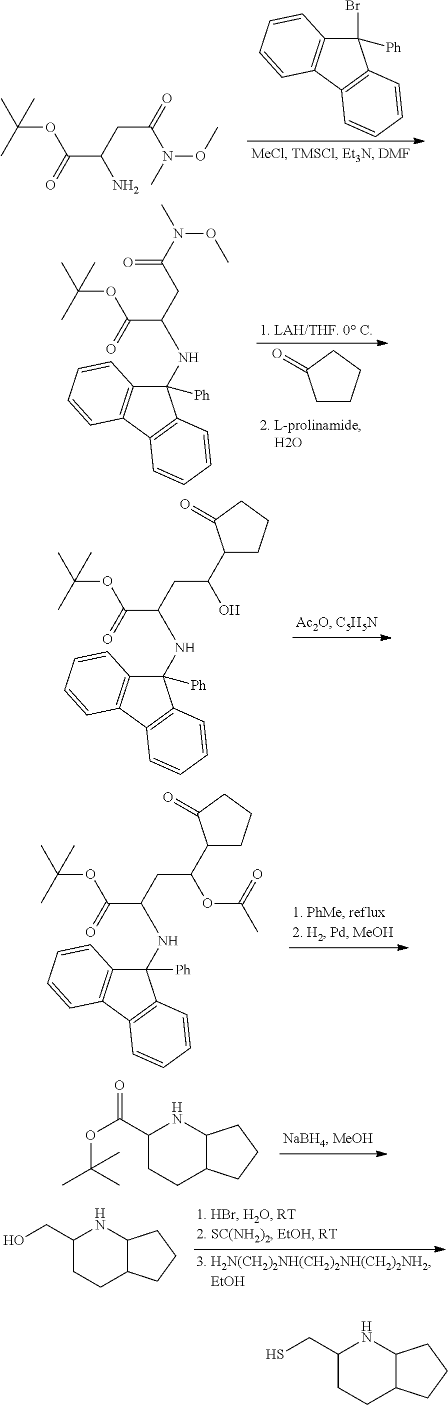

Method 43:

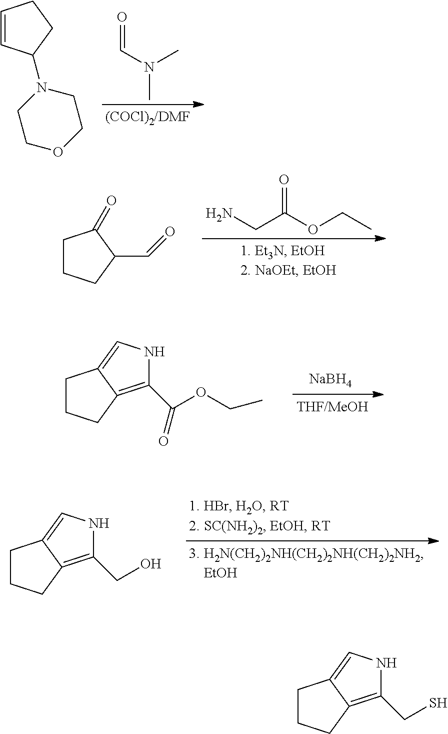

Method 44:

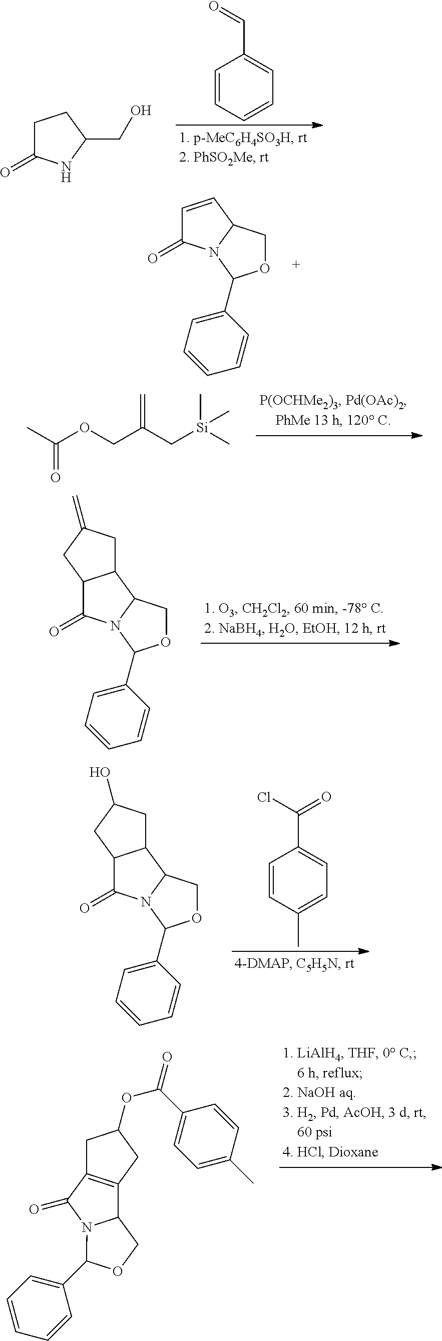

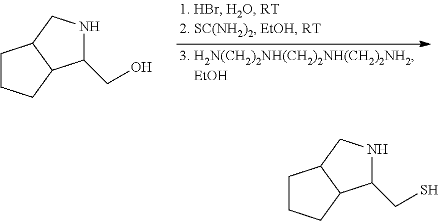 [SMILES: OCC1NCC2CCCC21 ; SCC1NCC2CCCC21]

Method 45:

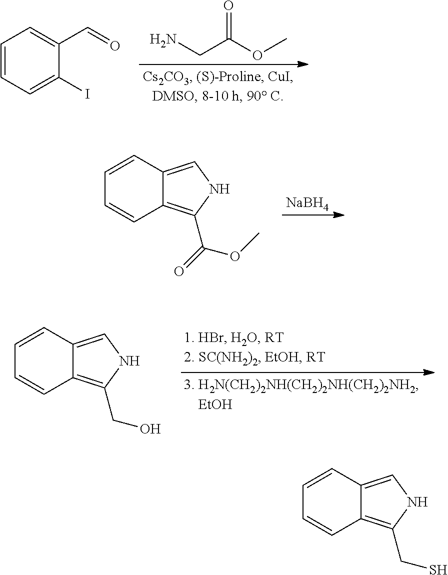

Method 46:

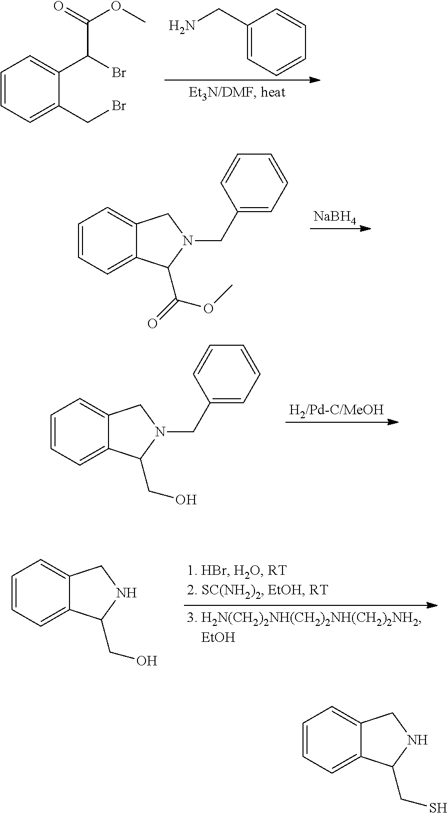

Method 47:

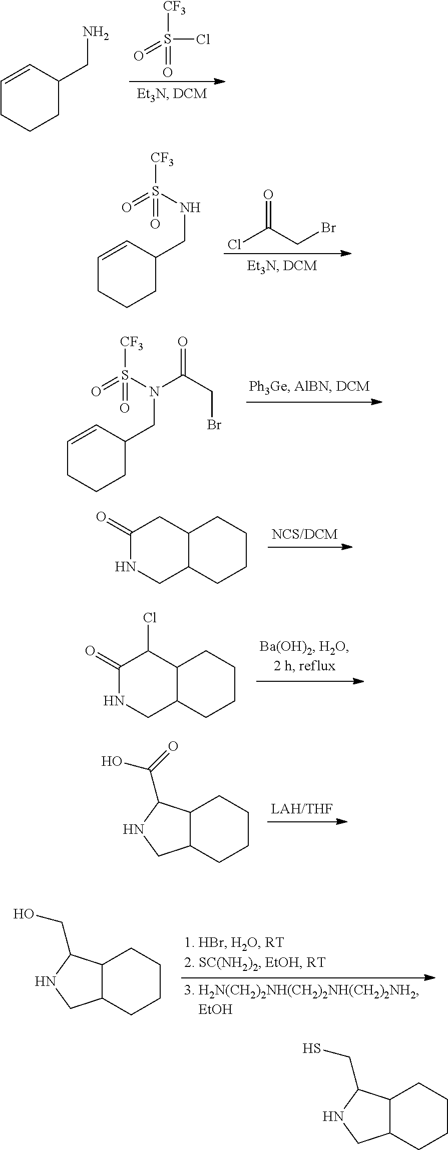

Method 48:

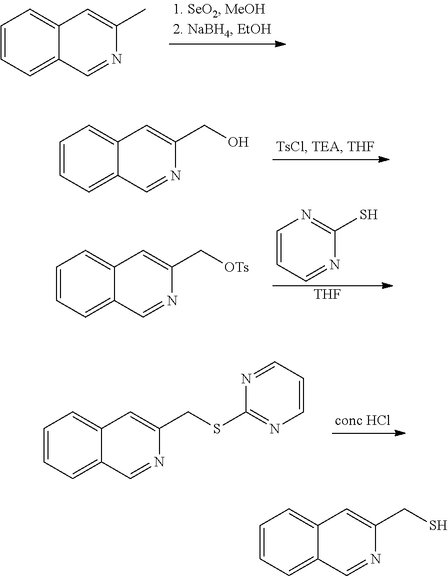

Method 49:

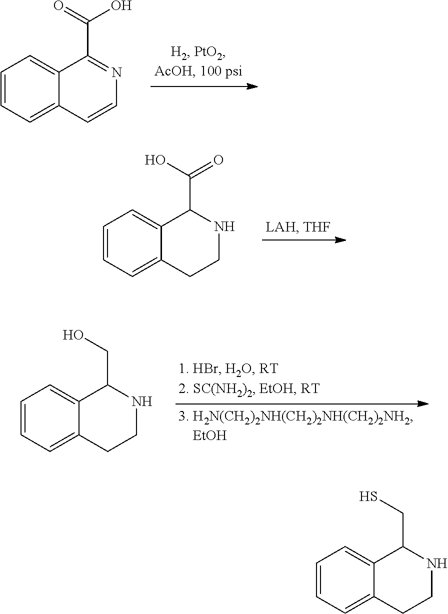 [SMILES: O=C(O)c1nccc2ccccc12 ; O=C(O)C1NCCc2ccccc21 ; OCC1NCCc2ccccc21 ; SCC1NCCc2ccccc21]

Method 50:

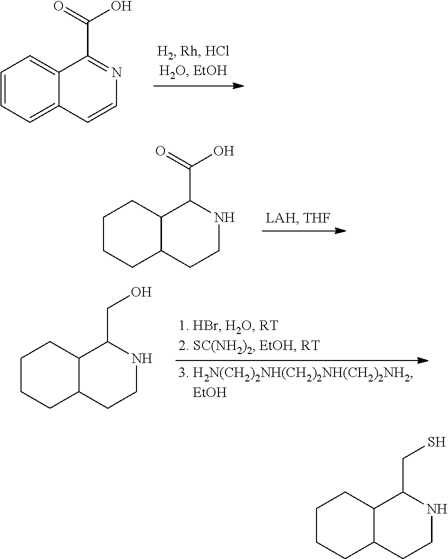

These same methods may be applied to the other aliphatic and aromatic ring frameworks to provide the desired first and second aspects and embodiments of the invention. Starting aliphatic or aromatic ring frameworks are known in the art and readily accessible from chemical sources such as Aldrich Chemical. Additional methods of synthesis are provided in the following examples. The procedures of these examples may be followed to append chemical substituents to the ring positions of the aliphatic or aromatic ring frameworks of the invention.

### EXAMPLES

General Methods. Chemicals, reagents and solvents were obtained from commercial sources and used as received. NMR spectra were obtained on a Varian Mercury-300 spectrometer in the indicated solvents. Analytical LCMS was run on Agilent LC/MSD 1200 Series ending with a mixture of acetonitrile and water containing 0.1% acetic acid. Preparative reverse phase HPLC was run using Water 2695 instrument with a YM 25 cm×50 mm column eluting with a mixture of acetonitrile and water containing 0.1% ammonium acetate.

### Example 1

### Synthesis of 8-F-2-Me-quinoline

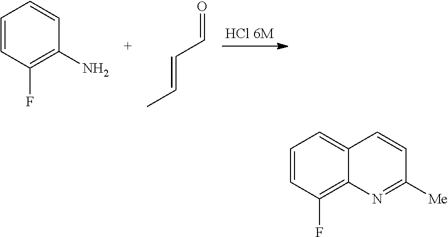

To a solution of 2-fluoroaniline (1 g, 9 mmol) in toluene (40 mL) at the room temperature were added aqueous HCl solution (6M, 12 mL) and but-2-enal (1.8 mmol). Then the heterogeneous mixture was stirred at 110° C. overnight. The aqueous layer was separated, neutralized to pH 9 and extracted with EtOAc (3×50 mL). The combined organic layers were washed with brine (10 mL) and dried over Na₂SO₄. The Na₂SO₄ was removed by filtration and the volatiles were removed under reduce pressure. Purification of the residue by a flash chromatography (Hex/EtOAc; 10/0 to 9/1) obtained the desired compound (0.6 g, 42% yield). LRMS (M+H⁺) m/z: cacld 162.06, found 162.26.

Other analogues such as 3-Me and 4-Me were prepared through similar procedures.

### Example 2

### Synthesis of 8-F-5-Me-quinoline

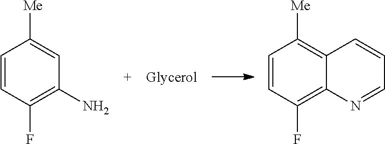 [SMILES: Cc1ccc(F)c(N)c1 ; Cc1ccc(F)c2ncccc12]

To a solution of aniline (500 mg, 4 mmol) in aqueous sulfuric acid solution (4 mL, 75% concentration) at the room temperature were added nitrobenzene (0.4 mL, 4 mmol) and glycerol (0.6 mL, 8 mmol), and then it was stirred at 150° C. for 2 hours. The resulting solution was cooled to the room temperature and diluted with water (50 mL), followed by extraction with EtOAc (3×50 mL). The combined organic layers were washed with brine (10 mL) and dried over Na₂SO₄. The Na₂SO₄ was removed by filtration and the volatiles were removed under reduce pressure. Purification of the residue by a flash chromatography (Hex/EtOAc; 98/2 to 80/20) obtained the desired compound (190 mg, 30% yield). LRMS (M+H⁺) m/z: cacld 162.06, found 162.26.

Other analogues such as 6-Me were prepared through similar procedures.

### Example 3

### Synthesis of 8-SBu^{t}-2-Me-quinoline

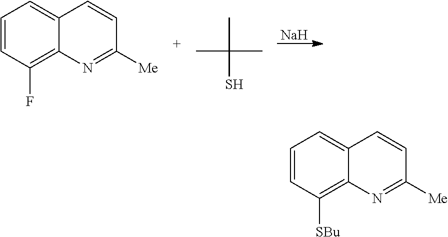

To a solution of the 8-F-2-Me-quinoline (500 mg, 3.1 mmol) in anhydrous DMF (50 mL) at the room temperature were added NaH (248 mg, 10 mmol) and t-butylthiol (0.7 mL, 6.2 mmol) under nitrogen atmosphere, and then it's stirred at 150° C. overnight. The resulting solution was cooled down and the solvents were removed under reduced pressure. Purification of the residue by a flash chromatography (Hex/EtOAc; 100/0 to 93/7) obtained the desired compound (0.6 g, 42% yield). LRMS (M+H⁺) m/z: calcd 232.11, found 232.36.

Other analogues were prepared through similar procedures.

### Example 4

### Synthesis of 8-SH-2-Me-quinoline

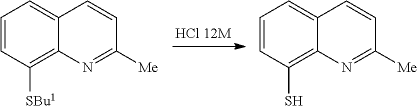

A solution of 8-SBu^{t}-2-Me-quinoline (40 mg) in aqueous HCl solution (11 mL, 35% concentration) was stirred at 110° C. for 12 hours. The resulting solution was cooled down and neutralized to pH 9, followed by extraction with EtOAc (3×50 mL). The combined organic layers were washed with brine (10 mL) and dried over Na₂SO₄. The Na₂SO₄ was removed by filtration and the volatiles were removed under reduce pressure. Purification of the residue by a flash chromatography (Hex/EtOAc: 100/0 to 80/20) obtained the desired compound (9 mg, 30% yield). LRMS (M+H⁺) m/z: cacld 176.05, found 176.25, ¹H NMR (CDCl3): □ 2.9 (s, 3H), 7.3 (m, 2H), 7.5 (m, 1H), 7.8. (m, 1H), 8.0 (m, 1H).

Other analogues were prepared through similar procedures.

### Example 5

### Synthesis of 8-F-4-CO₂H-quinoline

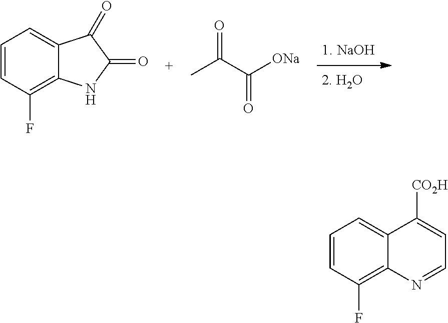

To a solution of 7-fluoroisatin (500 mg, 3.03 mmol) in water (10 mL) at the room temperature were added aqueous NaOH solution (2.5 mL, 5M, 12.5 mmol) and sodium pyruvate (400 mg, 3.66 mmol), and it's then placed in a microwave reactor (CEM discover S) at 110° C. for 10 mm. The resulting solution was cooled down and acidified to pH 2, and the dark solid was then collected. A suspension of the solid in water (2 mL) was then placed in a 10 mL microwave tube followed by stirring as 170° C. (or 280 psi) in a microwave reactor for 5 min. The desired product (brown solid, 340 mg, 60% yield) was then filtered off and collected. LRMS (M+H⁺) m/z: cacld 192.04, found 192.16.

### Example 6

### Synthesis of 8-SBu^{t}-2-CO₂H-quinoline:

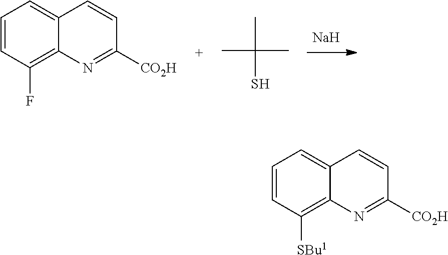

To a solution of the acid (420 mg, 2.19 mmol) in anhydrous DMF (40 mL) at room temperature were added NaH (175 mg, 6.6 mmol) and t-butylthiol (0.5 mL, 4.4 mmol) under nitrogen atmosphere, and then it's stirred at 150° C. overnight. The resulting solution was cooled down and the solvents were removed under reduced pressure. The residue was dissolved in water (10 mL) and acidified to pH 2, and the resulting solid was collected to obtain the desired compound (400 mg, 70% yield). LRMS (M+H⁺) m/z: calcd 262.08, found 262.34.

Other analogues were prepared through similar procedures.

### Example 7

### Synthesis of 8-SH-2-CO₂H-quinoline

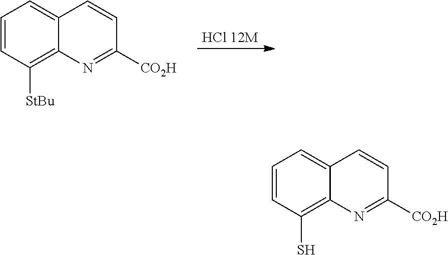

A solution of the acid derivatives (200 mg, 0.76 mmol) in aqueous HCl solution (18 mL, 36% concentration) was stirred at 110° C. for 12 hours. The crude was neutralized to pH 9 and washed with EtOAc (2×20 mL). The aqueous layer was then acidified to pH 2. The precipitate was filtered off and dried under vacuum to obtain the desired compound (9 mg, 30% yield). LRMS (M+H⁺) m/z: cacld 206.02, found 206.23. ¹H NMR (DMSO-d6): □ 7.6 (m, 1H), 7.8 (m, 1H), 7.9 (m, 1H), 8.2 (m, 1H), 8.6 (m, 1H).

Other analogues were prepared through similar procedures.

### Example 8

### Synthesis of 8-SH-3-CONHBn-quinoline

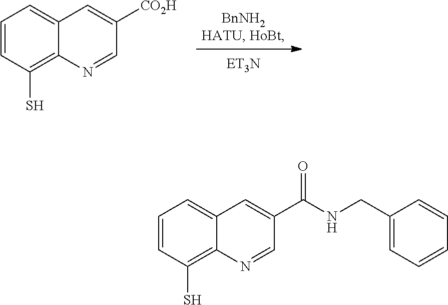 [SMILES: O=C(O)c1cnc2c(S)cccc2c1 ; "BnNH2HATU, HoBt," ; O=C(NCc1ccccc1)c1cnc2c(S)cccc2c1]

The starting compound 8-mercaptoquinoline-3-carboxylic acid was prepared according to the synthesis for compound A given below. To a solution of the acid (50 mg, 0.24 mmol) in anhydrous DMF (5 mL) at the room temperature were added HATU (111 mg, 0.29 mmol), HoBt (39 mg, 0.29 mmol), triethylamine (0.07 mL, 0.48 mmol) and phenylmethanamine (32 mg, 0.29 mmol) under nitrogen atmosphere, and then it's stirred at the same temperature for 3 hours. The resulting solution was diluted with water (20 mL) and extracted with EtOAc (3×50 mL). The combined organic layers were washed with brine (10 mL) and dried over Na₂SO₄. The Na₂SO₄ was removed by filtration and the volatiles were removed under reduce pressure. Purification of the residue by a flash chromatography (Hex/EtOAc; 100/0 to 80/20) obtained the desired compound (26 mg, 30% yield). LRMS (M+H⁺) m/z: cacld 295.08, found 295.37. ¹H NMR (DMSO-d6): 4.6 (d, 2H), 7.2 (m, 1H), 7.4 (m, 4H), 7.6 (m, 1H), 7.8 (m, 1H), 7.9 (m, 1H), 9.0 (s, 1H), 9.4 (s, 1H), 9.5 (m, 1H).

Other analogues were prepared through similar procedures.

### Example 9

### Synthesis of 8-SH-3-COGOMe-quinoline

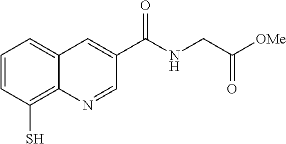 [SMILES: COC(=O)CNC(=O)c1cnc2c(S)cccc2c1]

It was prepared through the aforementioned procedure, LRMS (M+H⁺) m/z: cacld 277.06, found 277.31. ¹H NMR (DMSO-d6): 3.2 (s, 3H), 4.1 (s, 2H), 7.6 (m, 1H), 7.9 (m, 1H), 8.0 (m, 1H), 9.0 (s, 1H), 9.4 (s, 1H), 9.5 (m, 1H).

### Example 10

### Synthesis of 8-SH-3-COGGOBn-quinoline

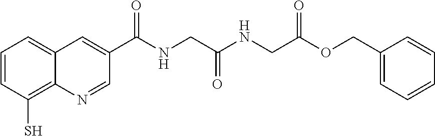 [SMILES: O=C(CNC(=O)c1cnc2c(S)cccc2c1)NCC(=O)OCc1ccccc1]

It was prepared through the aforementioned procedure. LRMS (M+H⁺) m/z: cacld 410.11, found 410.16. ¹H NMR (DMSO-d6): □□□m□□ 4.0 (m, 2H), 7.3 (m, 6H), 7.6 (m, 1H), 7.9 (m, 1H), 8.5 (m, 1H), 9.0 (s, 1H), 9.3 (m, 1H), 9.4 (s, 1H).

### Example 11

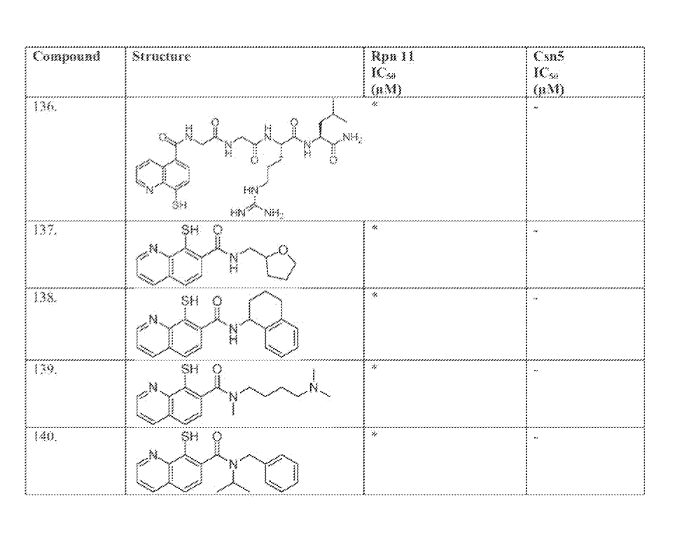

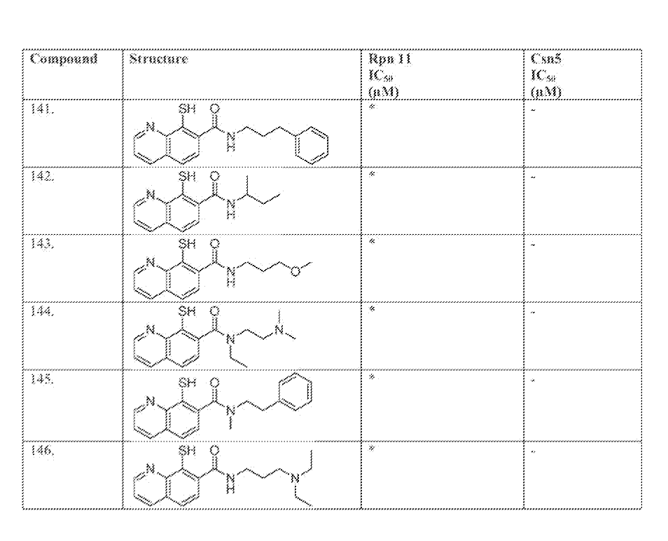

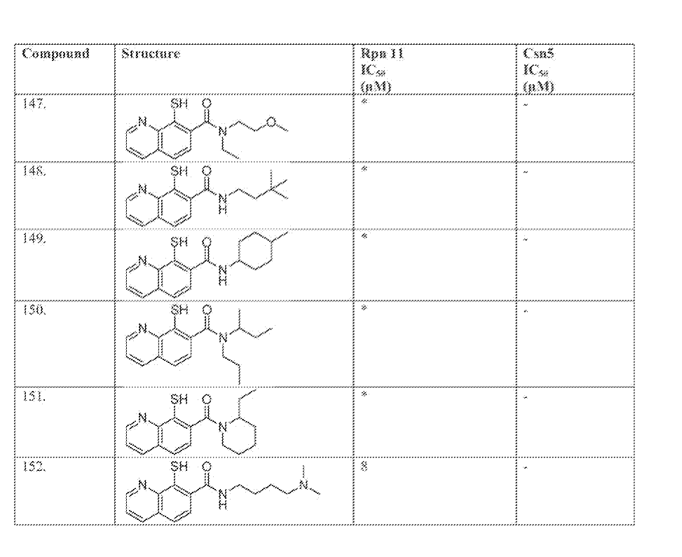

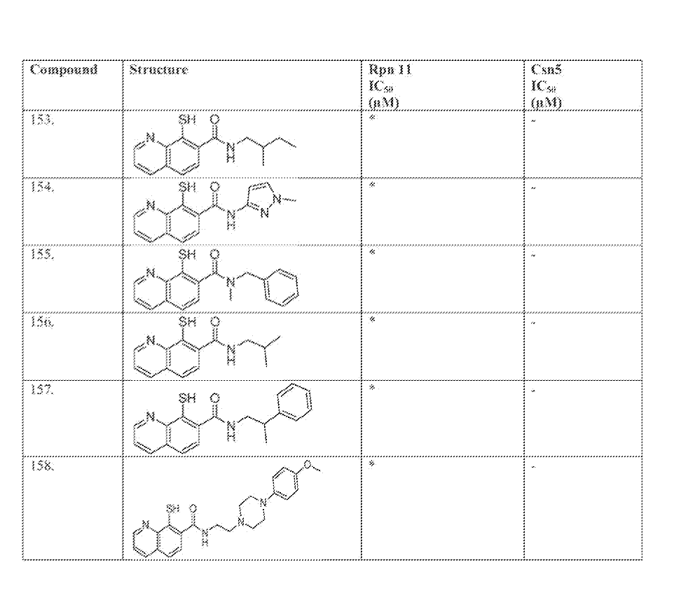

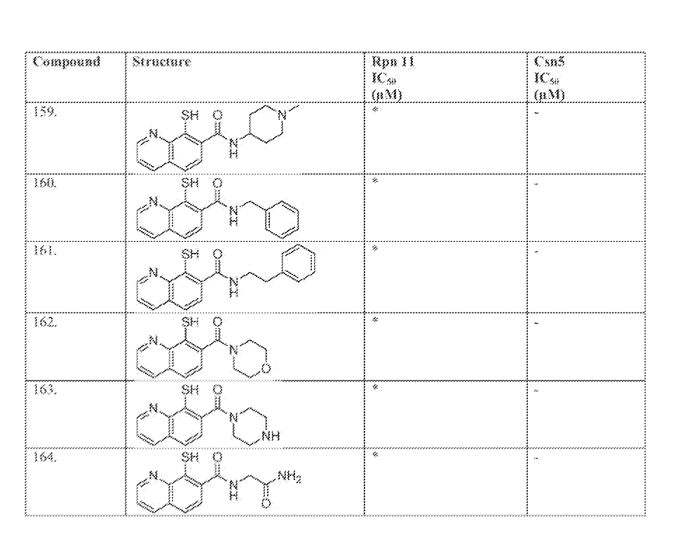

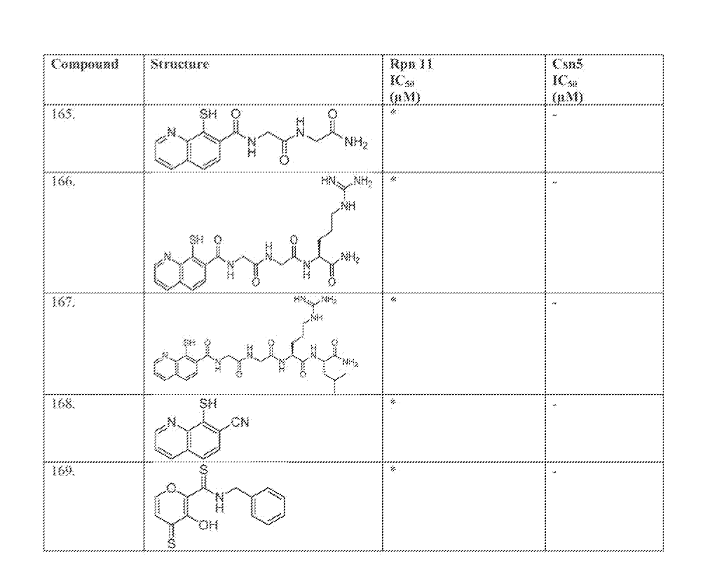

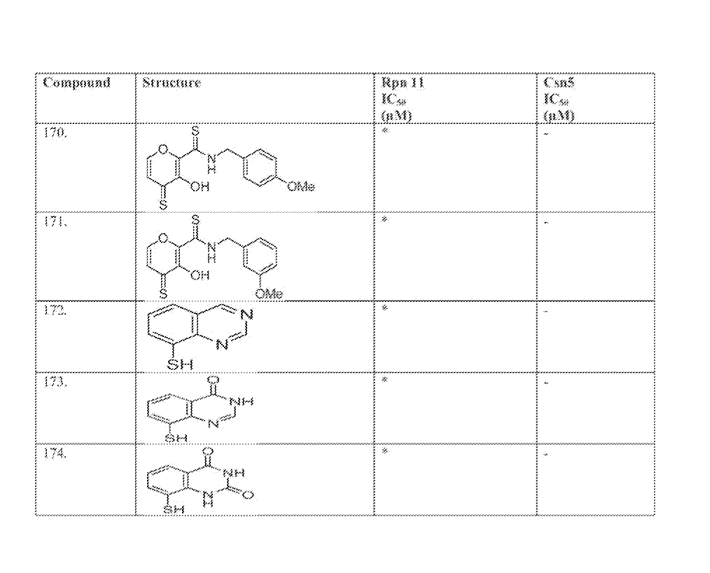

Chelation study with inorganic zinc ion. In the foregoing aspects 1-4 of the invention, the inhibitory compounds can be combined with a zinc cation to form a metal chelate with a K_{eq} less than 1 millimolar, as shown by a UV-visible absorption analysis of a solution of the organic molecule alone and the organic molecule completed with zinc cation in buffered aqueous medium. The UV-visible absorption analysis is conducted to determine absorption maxima for the organic molecule alone as λᵢ and the organic molecule in a saturated chelate with zinc cation as and the equilibrium constant K_{eq} being determine by monitoring the absorption at λᵢ and λ_{f} while titrating zinc cation into an aqueous solution of the organic molecule and calculating the equilibrium constant according to the equation K_{eq} equals the concentration of the organic molecule-Zn chelate divided by the multiple of the concentrations of the organic molecule alone and the free Zn cation. Following the foregoing procedure, test compounds were evaluated to determine appropriate zinc pharmacophores. As shown in FIG. 8, replacement of the nitrogen at position 1 with a carbon (B105, B116) or substitution of the —SH attached to position 8 with an —S—CH₃ (B101), —OH (B102), —NH₂ (B104) led to inactivation of B10, consistent with the hypothesis that B10 chelates enzyme-bound zinc via the N atom and SH group.

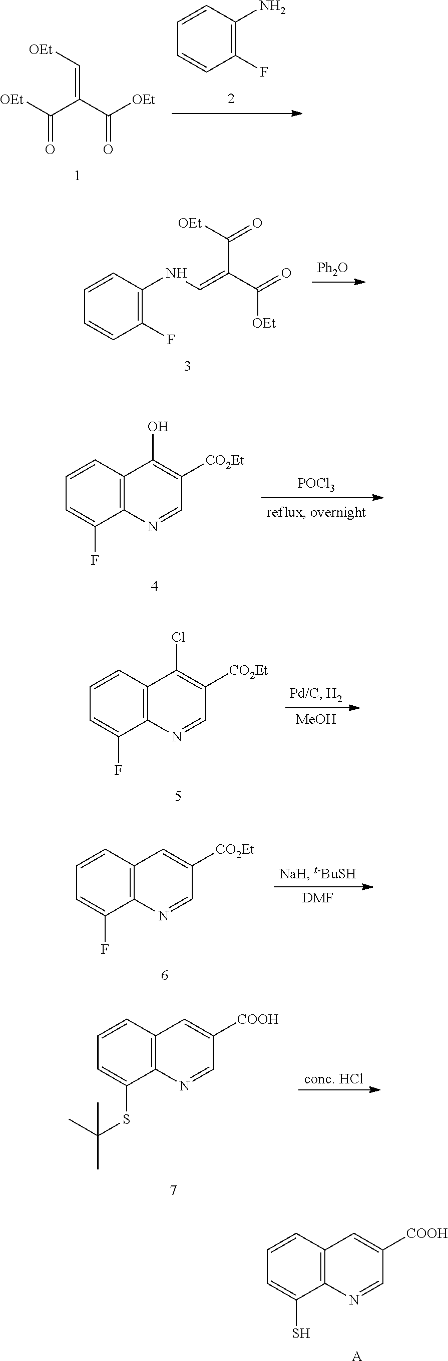

### Example 12

### Synthesis of Compound A

A mixture of compounds 1 (17.78 g, 0.16 mol) and 2 (37.36 g, 0.17 mol) was heated at 100° C. under atmosphere of nitrogen for 3 hours. The resulting solution was cooled down followed by dilution with hexane (100 mL). The mixture was then stirred at 60° C. for one hour and allowed to cool down to room temperature. The precipitated solid was filtered, washed with hexane (3×50 mL) and dried to yield compound 3 (42 g, 93.4%) as a white solid, which was used directly in the next step without further purification. LRMS (M+H⁺) m/z: cacld. 282.12; found 282.12.

A solution of compound 3 (42 g, 0.15 mol) in diphenylether (120 mL) was heated at 250° C. for 3 hours. The reaction mixture was cooled down followed by dilution with hexane (100 mL). The mixture was then stilled at 60° C. for one hour and allowed to cool down to room temperature. The precipitated solid was filtered, washed with hexane (3×50 mL) and dried to yield compound 4 (17 g, 48.4%) as a white solid, which was used in the next step without further purification. LRMS (M+H⁺) m/z: cacld. 236.06; found 230.06.

A solution of compound 4 (16 g, 68.0 mmol) in POCl₃ (120 g) was refluxed for 4 hours. The solvent was then removed under reduced pressure, and the residue was diluted with saturated aqueous NaHCO₃ solution (50 mL) and ethyl acetate (50 mL). The layers were separated, and the aqueous layer was extracted with and ethyl acetate (2×50 mL). The organic layers were combined and dried over Na₂SO₄. The Na₂SO₄ was removed by filtration, and the volatiles were removed under reduced pressure. The resulting residue was purified by flash chromatography using a mixture of hexane and ethyl acetate to provide compound 5 (16 g, 92.8%). ¹H NMR (CDCl₃): 9.54 (s, 1H), 8.43 (d, 1H), 7.99-7.84 (m, 2H), 4.57 (q, 2H), 1.50 (t, 3H).

To a 0° C. solution of compound 5 (15 g, 59.1 mmol) and Et₃N (8.7 mL) in MeOH (200 mL) was added 10% Pd/C (1 g). The reaction mixture was stirred at room temperature under hydrogen atmosphere (50 psi) for 3 hours. The Pd/C was then removed by filtration through Celtite and washed with THF (100 mL). The volatiles were removed under reduced pressure. The resulting residue was purified by flash chromatography using a mixture of hexane and ethyl acetate to provide compound 6 (10 g, 77%). LCMS (M+H⁺) m/z: cacld. 219.07, found 219.79. ¹H NMR (CDCl₃): 9.46 (d, 1H), 8.84 (t, 1H), 7.70 (dd, 1H), 7.55-7.49 (m, 2H), 4.47 (q, 2H), 1.44 (t, 3H).

To a 0° C. solution of compound 6 (5.0 g, 22.8 mmol) in anhydrous DMF (100 mL) were added NaH (1.8 g, 45.6 mmol) and t-BuSH (4.1 g, 45.6 mmol). The resulting mixture was stirred at room temperature overnight and then quenched with H₂O (120 mL). The resulting solution was extracted with ethyl acetate (3×100 mL). The combined organic layers were washed with brine, dried over Na₂SO₄ and filtrated. The solvents were removed under reduced pressure, and the residue was purified by silica chromatography (ethyl acetate:petroleum=2:3) to give compound 7 (4.8 g, 80%). ¹H NMR (DMSO-d6): 13.50 (br, 1H), 9.36 (d, 1H), 8.99 (d, 1H), 8.20 (d, 1H), 8.12 (d, 1H), 7.69 (t, 1H), 1.33 (s, 9H).

A solution of compound 7 (500 mg) in concentrated aqueous HCl acid (50 mL) was refluxed for 12 hours. It's then cooled down to room temperature and the solvent was removed under reduced pressure. Then the residue was dissolved in water (30 mL), its pH value was adjusted to 5.5 with saturated aqueous NaHCO₃ solution. The resulting solution was extracted with and ethyl acetate (2×100 mL). The organic layers were combined and dried over Na₂SO₄. The Na₂SO₄ was removed by filtration, and the volatiles were removed under reduced pressure. The resulting residue was purified by preparative HPLC to afford compound A (180 mg, 46% yield) as a pale yellow solid. LRMS (M+H⁺) m/z: cacld. 205.02, found 205.02; ¹H NMR (DMSO-d6): 9.41 (d, 1H), 9.05 (d, 1H), 8.06 (d, 1H), 7.88 (dd, 1H), 7.64 (t, 1H).

### Example 13

### Synthesis of Compound B

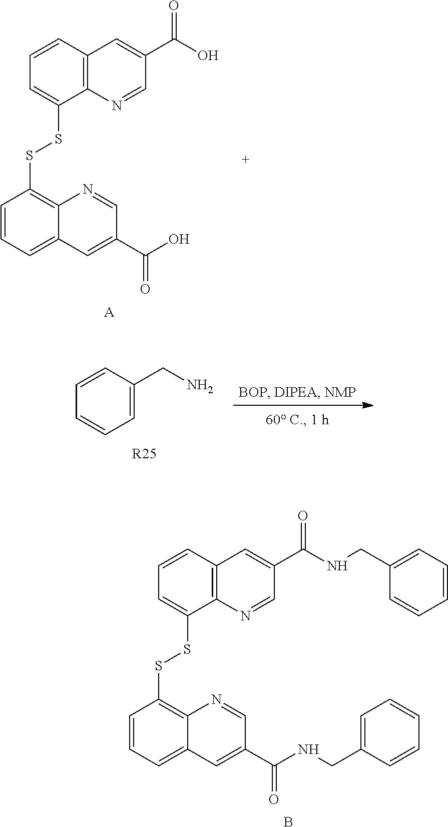

to a −5° C. mixture of compounds A (41 mg, 0.1 mmol) and R25 (64 mg, 0.6 mmol) in NMP (1 mL) were added BOP (133 mg, 0.3 mmol) and DIPEA (78 mg, 0.6 mmol). The reaction was kept at 60° C. for 1 hour, followed by dilution with EtOAc (50 mL) and brine (30 mL). The layers were separated, and the aqueous layer was extracted with EtOAc (2×100 mL). The organic layers were combined and dried over Na₂SO₄. Na₂SO₄ was removed by filtration, and the volatiles were removed under reduced pressure. The resulting residue was purified by preparative HPLC to obtain the pure product B (10 mg, 17%) as a white solid. LRMS (M+H⁺) m/z: cacld. 587.15, found 587.1; ¹H NMR (DMSO-d6): 9.46 (t, 2H), 9.41 (d, 2H), 8.95 (d, 2H), 7.95 (d, 2H), 7.85 (dd, 2H), 7.65 (t, 2H), 7.42-7.37 (m, 10H), 4.59 (d, 4H).

### Example 14

### Synthesis of Compound C

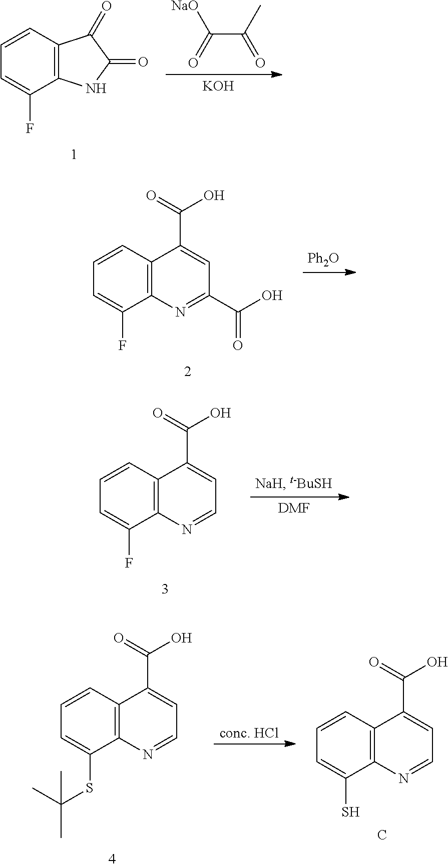

To a 0° C. solution of compound 1 (50 g, 0.3 mol) in aqueous KOH (concentration: 33%; 10 mL) was added 2-oxopropanoic acid sodium salt (50 g, 0.45 mol), and the resulting solution was stirred at 50° C. overnight. Then the mixture was cooled down to room temperature. The precipitated solid was collected and washed with the aqueous KOH solution (20 mL). The solid was then stirred in water (50 mL) and the remaining solid was filtrated off, and the combined aqueous solutions were neutralized with concentrated aqueous HCl acid to pH=2. The white precipitation was collected and dried in vacuum to give compound 2 (68 g, 96% yield), which was used in the next step without further purification. LRMS (M+H⁺) m/z: cacld. 236.03, found 236.03.

A solution of compound 2 (68 g, 0.29) in diphenyl ether (120 mL) was heated at 250° C. for 3 hours. The reaction mixture was cooled down to room temperature and diluted with hexane (100 mL). The mixture was then stirred at 60° C. for one hour and allowed to cool down to room temperature. The precipitated solid was filtered, washed with hexane (50 mL) and dried to yield compound 3 (48 g, 87% yield) as a white solid, which was used directly in the next step without further purification. LRMS (M+H⁺) m/z: cacld. 192.04; found 192.04, ¹H NMR (DMSO-d6): 14.04 (br, 1H), 9.09 (d, 1H), 8.51 (dd, 1H), 8.03 (d, 1H), 7.75-7.64 (m, 2H).

To a 0° C. solution of compound 3 (48 g, 0.20 mol) in anhydrous DMF (500 mL) were added NaH (23.5 g, 0.98 mol) and t-BuSH (54 g, 0.6 mol). The resulting mixture was stirred at 150° C. overnight and then cooled down to room temperature and quenched with water (120 mL). The resulting solution was extracted with ethyl acetate (3×50 mL). The combined organic layers were washed with brine, dried over Na₂SO₄ and filtrated. The solvents were removed under reduced pressure, and the residue was purified by silica chromatography (ethyl acetate:petroleum=2:3) to give compound 4 (35 g, 67% yield). ¹H NMR (DMSO-d6): 13.95 (br, 1H), 9.09 (d, 1H), 8.63 (dd, 1H), 8.07 (dd, 1H), 7.93 (d, 1H), 7.70 (t, 1H), 1.33 (s, 9H).

A solution of compound 4 (2 g, 7.7 mmol) in concentrated aqueous HCl acid (50 mL) was refluxed for 12 hours. It's then cooled down to room temperature and the solvent was removed trader reduced pressure. Then the residue was dissolved in 30 mL water and then its pH value was adjusted to 5.5 with saturated aqueous NaHCO₃ solution. It was extracted with and ethyl acetate (2×100 mL) and the organic layers were combined and dried over Na₂SO₄. The Na₂SO₄ was removed by filtration, and the volatiles were removed under reduced pressure. The resulting residue was purified by preparative HPLC to afford compound C (0.8 g, 51%) as a pale yellow solid. LRMS (M+H⁺) m/z: cacld. 408.02, found 408.0; ¹H NMR (DMSO-d6): 14.1-13.9 (m, 1H), 9.15 (d, 1H), 8.53 (d, 1H), 8.06 (d, 1H), 7.81 (d, 1H), 7.63 (t, 1H).

### Example 15

### Synthesis of Compound D

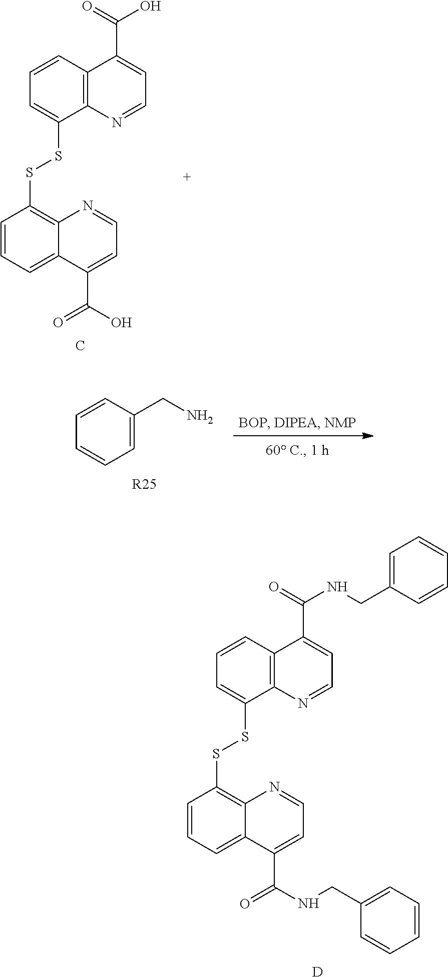

To a −5° C. mixture of compounds C (41 mg, 0.1 mmol) and R25 (64 mg, 0.6 mmol) in NMP (1 mL) were added BOP (133 mg, 0.3 mmol) and DIPEA (78 mg, 0.6 mmol). The reaction was kept at 60° C. for 1 hour, followed by dilution with EtOAc (50 mL) and brine (30 mL). The layers were separated, and the aqueous layer was extracted with EtOAc (2×50 mL). The organic layers were combined and dried over Na₂SO₄. The Na₂SO₄ was removed by filtration, and the volatiles were removed under reduced pressure. The resulting residue was purified by preparative HPLC to obtain the pure product D (8 mg, 14%) as a white solid. LRMS (M+H⁺) m/z: cacld. 587.15, found 587.1; ¹H NMR (DMSO-d6): 9.38 (t, 2H), 9.11 (d, 2H), 7.94 (d, 2H), 7.78-7.74 (m, 4H), 7.57 (t, 2H), 7.42-7.26 (m, 10H), 4.57 (d, 4H).

### Example 16

### Synthesis of Compound E

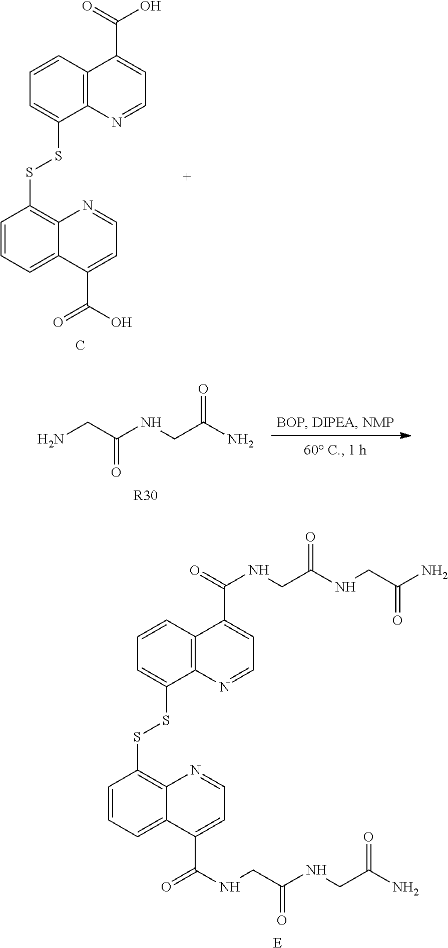

To a −5° C. mixture of compounds C (41 mg, 0.1 mmol) and R30 (64 mg, 0.6 mmol) in NMP (1 mL) were added BOP (133 mg, 0.3 mmol) and DIPEA (78 mg, 0.6 mmol). The reaction was kept at 60° C. for 1 hour, followed by dilution with EtOAc (50 mL) and brine (30 mL). The layers were separated, and the aqueous layer was extracted with EtOAc (2×50 mL). The organic layers were combined and dried over Na₂SO₄. The Na₂SO₄ was removed by filtration, and the volatiles were removed under reduced pressure. The resulting residue was purified by preparative HPLC to obtain the pure product E (7.1 mg, 12%) as a white solid. LRMS (M+H⁺) m/z: cacld. 635.14, found 635.2; ¹H NMR (DMSO-d6): 9.38 (t, 2H), 9.11 (d, 2H), 7.94 (d, 2H), 7.78-7.74 (m, 4H), 7.57 (t, 2H), 7.42-7.26 (m, 10H), 4.57 (d, 4H).

### Example 17

### Synthesis of Compounds F and G

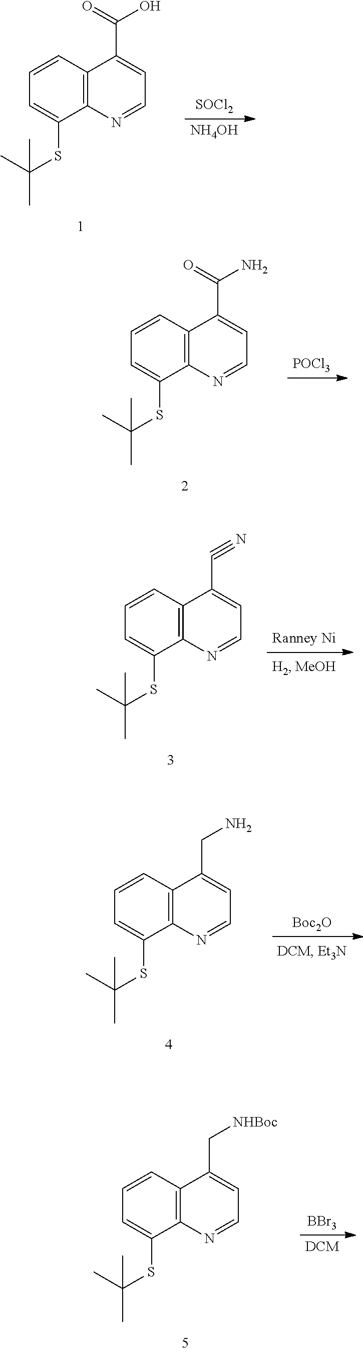

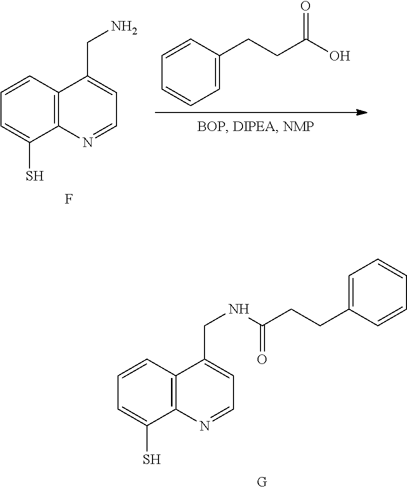

To a 0° C. suspension of compound 1 (10 g, 38.3 mmol) in DCM (100 mL) was added SOCl₂ (10 mL), and the resulting mixture was refluxed for 2 hours. Then the reaction solution was cooled down to room temperature and poured into NH₃.H₂O (200 mL). The resulting mixture was stirred for another half hour and then diluted with EtOAc (100 mL). The layers were separated, and the aqueous layer was extracted with EtOAc (2×100 mL). The organic layers were combined and dried over Na₂SO₄. The Na₂SO₄ was removed by filtration, and the volatiles were removed under reduced pressure. The resulting residue was washed with dimethylether to obtain pure compound 2 (8.5 g, 85%) as a yellow solid. ¹H NMR (DMSO-d6): 9.02 (d, 1H), 8.26 (br, 1H), 8.18 (dd, 1H), 8.04 (dd, 1H), 7.94 (br, 1H), 7.65 (t, 1H), 7.58 (d, 1H), 1.27 (s, 9H).

To a 0° C. of compound 2 (8.5 g. 32.7 mmol) in dioxane (100 mL) was added POCl₃ (10 mL), the resulting solution was refluxed for 2 hours. The solvent was then removed under reduced pressure, and the residue was diluted with saturated aqueous NaHCO₃ solution (50 mL) and ethyl acetate (50 mL). The layers were separated, and the aqueous layer was extracted with and ethyl acetate (2×100 mL). The organic layers were combined and dried over Na₂SO₄. The Na₂SO₄ was removed by filtration, and the volatiles were removed under reduced pressure. The resulting residue was purified by flash chromatography using a mixture of hexane and ethyl acetate to provide compound 3 (6.5 g, 82% ¹H NMR (CDCl₃): 9.17 (d, 1H), 8.21 (dd, 1H), 8.17 (dd, 1H), 7.76 (d, 1H), 7.73 (t, 1H), 1.38 (s, 9H).

To a solution of compound 3 (6.5 g, 26.9 mmol) in MeOH (100 mL) at room temperature was added Ranney Ni (20 g). The reaction mixture was stirred at room temperature under hydrogen atmosphere. The Ranney Ni was then removed by filtration through Celtite and washed with MeOH (40 mL) once the reaction completed. The volatiles were removed under reduced pressure. The resulting residue was dried to provide compound 4, which was used in next step without further purification. LRMS (M+H⁺) m/z: cacld. 247.12, found 247.12.

To a 0° C. mixture of aforementioned intermediate 4 and and Et₃N (18.7 mL, 0.13 mol) in DCM (20 mL) was added Boc₂O (11.7, 53.8 mmol). The reaction was stirred at room temperature for 4 hour, followed by dilution with DCM (50 mL), washed with aqueous HCl acid (0.2 M, 20 mL) and brine (30 mL). The layers were separated, and the combined aqueous layers were extracted with DCM (2×50 mL). The organic layers were combined and dried over Na₂SO₄. The Na₂SO₄ was removed by filtration, and the volatiles were removed under reduced pressure. The resulting residue was purified by flash chromatography using a mixture of hexane and ethyl acetate to provide compound obtain the pure product 5 (3 g, 32%, yield of two steps) as a white solid. LRMS (M+H⁺) m/z: cacld. 635.14, found 635.2; ¹H NMR (CDCl₃): 9.00 (d, 1H), 8.05-7.99 (m, 2H), 7.54 (t, 1H), 7.36 (d, 1H), 4.98 (br, 1H), 4.81. (d, 2H), 1.48 (s, 9H), 1.37 (s, 9H).

To a 0° C. solution of aforementioned intermediate 5 (3 g, 8.7 mmol) in DCM (30 mL) was added BBr₃ (1.65 mL, 17.4 mmol). The reaction was stirred at room temperature for 30 min, coaled down 0° C. and then quenched with MeOH (50 mL). The resulting mixture was then stirred at room temperature for extra 10 min. The solvents were removed under reduced pressure. The resulting residue was washed with ethyl acetate to obtain the product F (1.6 g, 68%) as a yellow solid. LRMS (M+H⁺) m/z: 379.09, found 379.1; ¹H NMR (CDCl₃): 8.98 (d, 2H), 7.89 (dd, 2H), 7.81 (dd, 2H), 7.58 (d, 2H), 7.42 (t, 2H), 4.39 (s, 4H).

To a −5° C. solution of compound F (45 mg, 0.12 mmol) and 3-phenylpropanoic acid (37 mg, 0.25 mmol) in NMP (1 mL) were added BOP (133 mg, 0.3 mmol) and DIPEA (78 mg, 0.6 mmol). The reaction was kept at 60° C. for 1 hour, followed by dilution with EtOAc (50 mL) and brine (30 mL). The layers were separated, and the aqueous layer was extracted with EtOAc (2×50 mL). The organic layers were combined and dried over Na₂SO₄. The Na₂SO₄ was removed by filtration, and the volatiles were removed under reduced pressure. The resulting residue was purified by preparative HPLC to obtain the pure product G (31.9 mg, 41%) as a white solid. LRMS (M+H⁺) m/z: cacld. 642.21, found 643.3; ¹H NMR (DMSO-d6): 8.88 (d, 2H), 8.54 (t, 2H), 7.96 (d, 2H), 7.72 (d, 2H), 7.53 (t, 2H), 7.30-7.20 (m, 12H), 4.75 (d, 4H), 2.89 (t, 4H), 2.55 (t, 4H).

### Example 18

### Synthesis of Compounds H and I

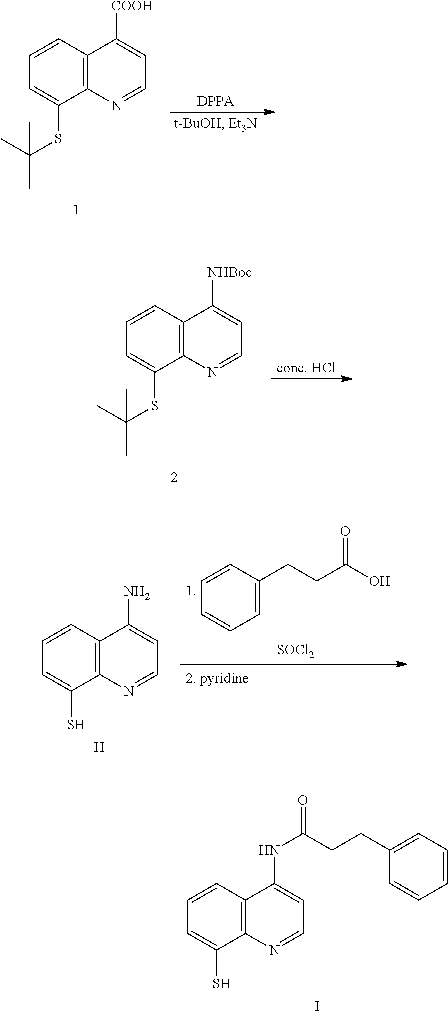

To a −5° C. solution of compound 1 (5 g, 19 mmol) in t-BuOH (50 mL) were added Et₃N (8.0 mL, 57 mmol) and DPPA (6.2 mL, 28.5 mmol). The reaction was kept at 100° C. overnight and then cooled down to room temperature. The solvents were removed and the residue was diluted with EtOAc (50 mL) and saturated aqueous NaHCO₃ solution (30 mL). The layers were separated, and the aqueous layer was extracted with EtOAc (2×100 mL). The organic layers were combined and dried over Na₂SO₄. The Na₂SO₄ was removed by filtration, and the volatiles were removed under reduced pressure. The resulting residue was purified by flash chromatography using a mixture of hexane and ethyl acetate to provide the pure product the pure product 2 (4 g, 64%) as a white solid. ¹H-NMR (CDCl₃): 8.92 (d, 1H), 8.10 (d, 1H), 8.01 (dd, 1H), 7.78 (dd, 1H), 7.50 (t, 1H), 1.58 (s, 9H), 1.39 (s, 9H).

A solution of compound 2 (4 g, 12.1 mmol) in concentrated aqueous HCl acid (50 mL) was refluxed for 12 hours. It's then cooled down and the solvent was removed under reduced pressure. Then the residue was dissolved in water (30 mL) and then its pH value was adjusted to 5.5 with saturated aqueous NaHCO₃ solution. It was extracted with and ethyl acetate (2×200 mL). The organic layers were combined and dried over Na₂SO₄. The Na₂SO₄ was removed by filtration, and the volatiles were removed under reduced pressure. The resulting residue was purified by preparative HPLC to afford compound H (1.8 g, 70%, HCl salt) as a yellow solid, LRMS (M+H⁺) m/z: cacld 351.07, found 351.1; ¹H-NMR (DMSO-d6): 13.14 (br, 1H) 9.45 (br, 2H), 8.67 (d, 1H), 8.33 (d, 1H), 7.51-7.42, (m, 2H), 6.97 (d, 1H).

A solution of 3-phenylpropanoic acid (300 mg, 2 mmol) in SOCl₂ (10 mL) was refluxed for 2 hours, it was then cooled down to room temperature and the solvent was removed under reduced pressure. To a −5° C. solution of compound H (0.1 g, 0.47 mmol) in pyridine (5 mL) was added the aforementioned acidic chloride. The reaction was kept at 50° C. overnight. The solvent was then removed and the residue was diluted with EtOAc (50 mL) and brine (30 mL). The layers were separated, and the aqueous layer was extracted with EtOAc (2×100 mL). The organic layers were combined and dried over Na₂SO₄. The Na₂SO₄ was removed by filtration, and the volatiles were removed under reduced pressure. The resulting residue was purified by preparative HPLC to obtain the pure product 1 (40 mg, 23%) as a yellow solid. LRMS (M+H⁺) m/z: cacld 615.18, found 615.2; ¹H-NMR (DMSO-d6): 10.21 (br, 1H), 8.89 (d, 1H), 8.23 (d, 1H). 8.12 (d, 1H), 7.74 (d, 1H), 7.52 (t, 1H), 7.31-7.19 (m, 5H), 2.99-2.93 (m, 4H).

### Example 19

### Synthesis of Compound J

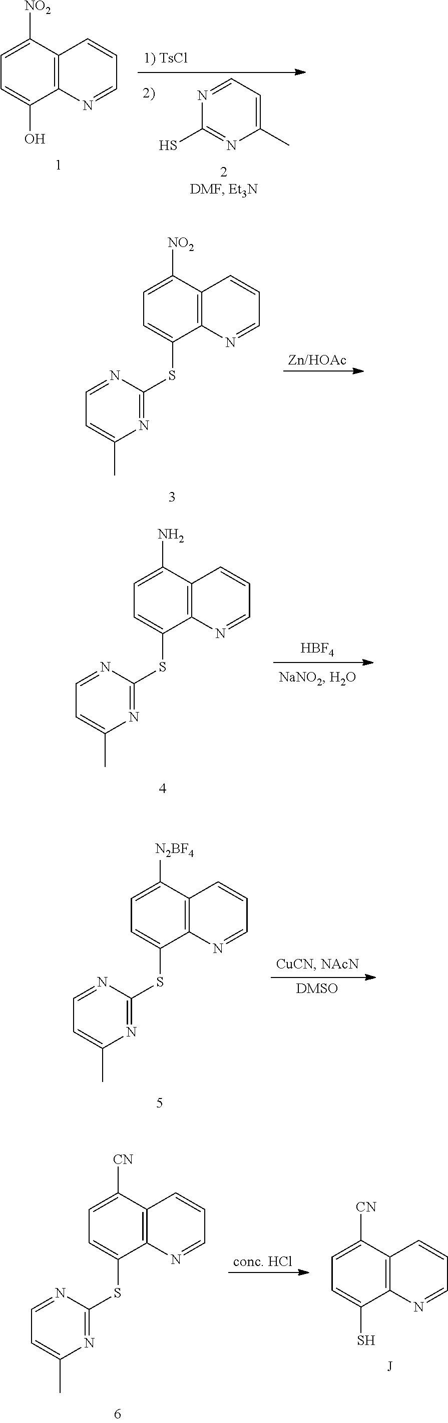

To a 0° C. solution of compound 1 (150 g, 0.79 mol) and Et₃N (239 g, 2.37 mol) in DMF (1.5 L) was added TsCl (150 g, 0.79 mol). The reaction was stirred at room temperature overnight and then poured into water (2 L). The precipitated solid was collected by filtration, dried to obtain crude Ts-protected intermediate (260 g) as a yellow solid, which was used in the next step without further purification.

To an aforementioned intermediate (260 g, 0.75 mol) and compound 2 (122 g, 0.75 mol) in DMF (2.5 L) at room temperature was added Et₃N (1.52 g, 1.50 mol). The reaction was stirred at room temperature overnight and the resulting mixture was poured into cold water (4 L). The precipitated solid was collected by filtration, dried to obtain a yellow solid (180 g, crude product). The crude product was dissolved in chloroform. (5.0 L) and washed aqueous sodium hydroxide solution (2×2.5L, 1M) and brine (2×2.5 L). The combined organic layers were combined and dried over Na₂SO₄. The Na₂SO₄ was removed by filtration, and the volatile were removed under reduced pressure. The resulting residue was purified by flash-chromatography using a mixture of hexane and ethyl acetate to provide compound 2 (140 g, 60%) as a yellow solid. ¹H-NMR (CDCl₃): 9.09 (dd, 1H), 9.01 (dd, 1H), 8.47 (d, 1H), 8.40-8.37 (m, 2H), 7.67 (dd, 1H), 6.96 (d, 1H), 2.49 (s, 3H).

To a solution of compound 3 (75 g, 0.25 mol) in in HOAc (1.4 L) was added zinc powder (73.6 g, 1.13 mol). The reaction mixture was stirred at room temperature for a hours and then added slowly into a 0° C. aqueous potassium hydroxide solution (12 L, 2 N). The resulting solid was collected by filtration, and then triturated with DCM (3×5 L). The combined organic layers were combined and dried over Na₂SO₄. The Na₂SO₄ was removed by filtration, and the volatiles were removed under reduced pressure. The resulting residue was dried to yield compound 4 (38 g, 57%) as a brown solid. ¹-NMR (DMSO-d6): 8.76 (dd, 1H), 8.59 (dd, 1H), 8.19 (d, 1H), 7.75 (d, 1H), 7.39 (dd, 1H), 6.94 (d, 1H), 6.73 (d, 1H), 6.46 (s, 2H), 2.29 (s, 3H).

To a 0° C. solution of intermediate 4 (29.1 g, 0.11 mol) and tetrafluoroboric acid (236.7 mL, concentration: 48%) in water (167 mL) was slowly added a solution of sodium nitrite (7.6 g, 0.11 mol) in water (167 mL). The resulting solution was stirred at the same temperature for one hour, and the precipitated solid was collected by filtration, washed with water (400 mL), diether ether (400 mL) and dried to yield compound 5 (29 g, 73%) as a brown solid. ¹-NMR (DMSO-d6): 9.28 (dd, 1H), 9.10 (d, 1H), 9.00 (dd, 1H), 8.82 (d, 1H), 8.75 (d, 1H), 8.17 (dd, 1H), 7.48 (d, 1H), 2.55 (s, 3H).

A mixture of copper cyanide (12.1 g, 0.14 mol) and sodium cyanide (8.6 g, 0.18 mol) in DMSO (100 mL) was stirred at room temperature overnight till it becomes homogenous. Intermediate 5 (10 g, 0.027 mol) was then added in small portions to maintain its temperature at 20° C. The resulting reaction mixture was stirred at the same temperature for another 30 min followed by dilution with water (180 mL) and ethyl acetate (200 mL). The layers were separated, and the aqueous layer was extracted with EtOAc (2×100 mL). The organic layers were combined and dried over Na₂SO₄. The Na₂SO₄ was removed by filtration, and the volatiles were removed under reduced pressure. The resulting residue was purified by flash chromatography using a mixture of hexane and ethyl acetate to to obtain the pure product 6 (3 g, 40% yield) as a white solid. ¹H-NMR (CDCl₃): 9.09 (dd, 1H), 9.01 (dd, 1H), 8.47 (d, 1H), 8:40-8.37 (m, 2H), 7.68 (dd, 1H), 6.96 (d, 1H), 2.47 (s, 3H).

A solution of compound 6 (2.6 g, 9.35 mmol) in aqueous HCl acid (300 mL, 6N) was refluxed for 6 hours. The resulting reaction mixture was cooled down and kept in freezer overnight. The precipitated solid was collected by filtration and dried to afford target compound J (1.5 g, 79%) as a yellow solid. LRMS (M+H⁺) m/z: cacld. 409.02, found 409.03; ¹H-NMR (DMSO-d6): 9.08 (d, 1H), 9.00 (d, 1H), 8.11 (d, 1H), 7.87 (d, 1H), 7.81 (t, 1H).

### Example 20

### Synthesis of Compound L

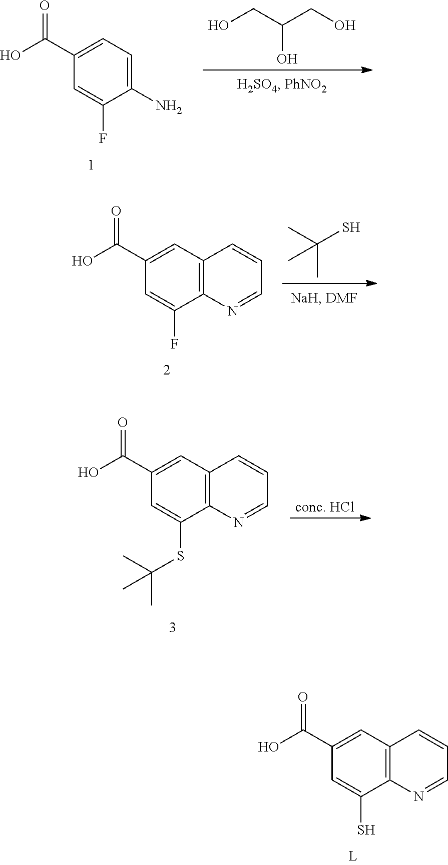

To a 0° C. mixture of glycerol (36.8 g, 0.4 mol), 4-amino-3-fluorobenzoic acid 1 (15.5 g, 0.1 mol) and nitrobenzene (7.4 g, 0.06 mol) was concentrated sulfuric acid (17.4 mL). The mixture was then stirred at 150° C. for 3 hours. The resulting solution was cooled down to the room temperature and diluted with water (100 mL). It was then neutralized with saturated aqueous NaHCO₃ solution to adjust pH=4. The precipitated solid was collected by filtration and washed with water (20 mL) and methanol (20 mL), the residue was dried to obtain the crude product 2 (7 g, 37%), which was used in the next step without further purification. LRMS (M−H⁺) m/z: cacld. 190.04, found 190.01; ¹H-NMR (DMSO-d6): 13.45 (br, 1H), 9.07 (dd, 1H), 8.66 (d, 1H), 8.53 (s, 1H), 7.93 (dd, 1H), 7.74 (dd, 1H).

To a solution of the 8-fluoroquinoline-6-carboxylic acid 2 (1.5 g, 8 mmol) in anhydrous DMF (15 mL) at room temperature were added NaH (1.3 g, 32 mmol) and 5-butylthiol (2.9 g, 32 mmol) under nitrogen atmosphere, and then the mixture was stirred at 110° C. overnight. The resulting solution was cooled down to room temperature and the solvents were removed under reduced pressure. The residue was purified by a flash chromatography (ethyl acetate:petroleum=1:1) to obtain the desired compound 3 (720 mg, 34%). LRMS (M−H⁺) m/z: cacld 260.08, found 260.1. ¹H-NMR (DMSO-d6): 8.96 (s, 1H), 8.56-8.43 (m, 3H), 7.54-7.53 (m, 1H), 1.24 (s, 9H).

A solution of compound 3 (1.2 g, 4.6 mmol) in concentrated aqueous HCl solution (60 mL) was stirred at 120° C. for 16 hours. The resulting solution was cooled down to room temperature and the solvents were removed under reduced pressure. The resulting residue was washed with MeOH and ethyl acetate (1/1, 50 mL) and then the solid was collected by filtration to yield the desired compound L (650 mg, 59% yield) as a pale yellow solid. LRMS (M+H⁺) m/z: cacld. 206.02, found 206.02; ¹H-NMR (CD₃OD): 8.99 (dd, J₁=4.5 Hz, J₂=1.5 Hz, 1H), 8.46 (dd, J₂=8.4 Hz, J₂=1.8 Hz, 1H), 8.38 (d, J=1.8 Hz, 1H), 8.28 (d, J=1.5 Hz, 1H), 7.63 (dd, J₁=4.5 Hz, J₂=8.4 Hz, 1H).

### Example 21

### Synthesis of Compound N

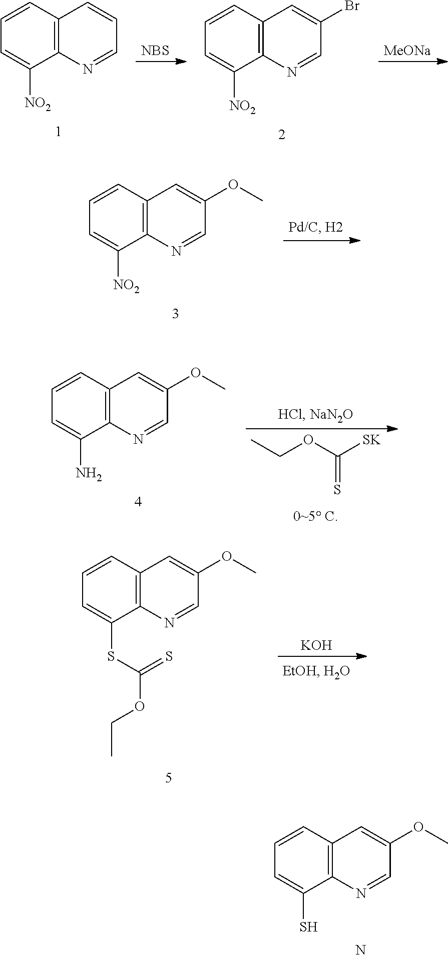

To a 0° C. solution of compound 1 (5 g, 28.9 mmol) in AcOH (50 mL) was added NBS (5.1 g, 28.9 mmol). The reaction was kept at 60° C. overnight and then cooled down to room temperature. The solvents were removed and the residue was diluted with EtOAc (50 mL) and water (30 mL). The layers were separated, and the aqueous layer was extracted with EtOAc (2×100 mL). The combined organic layers were washed with saturated aqueous NaHCO₃ solution (30 mL) and dried over Na₂SO₄. The Na₂SO₄was removed by filtration, and the volatiles were removed under reduced pressure. The resulting residue was purified by flash chromatography using a mixture of hexane and ethyl acetate to provide the pure product the pure product 2 (3 g, 41%) as a yellow solid.

To a 0° C. solution of compound 2 (1 g, 3.97 mmol) in DMF (10 mL) was added NaOMe (0.32 g, 5.96 mmol). The reaction was kept at room temperature for 5 hours and diluted with diethyl ether (50 mL) and water (30 mL). The layers were separated, and the aqueous layer was extracted with diethyl ether (2×50 mL). The combined organic layers were washed with saturated aqueous NaHCO₃ solution (30 mL) and dried over Na₂SO₄. The Na₂SO₄ was removed by filtration, and the volatiles were removed under reduced pressure. The resulting residue was purified by flash chromatography using a mixture of hexane and ethyl acetate to provide the pure product the pure product 3 (0.5 g, 62%) as a yellow solid). LRMS (M+H⁺) m/z: cacld. 204.06, found 204.06.

To a solution of compound 3 (0.5 g, 2.46 mmol) in MeOH (20 mL) at room temperature was added Pd/C (50 mg, 10%, wet). The reaction mixture was stirred at room temperature under hydrogen atmosphere for 5 hours. The Pd/C was then removed by filtration through Celtite and washed with MeOH (20 mL). The volatiles were removed under reduced pressure. The resulting residue was dried to provide compound 4 (0.4 g, 92%), which was used in next step without further purification. LRMS (M+H⁺) m/z: cacld. 174.08, found 174.08.

To a 0° C. solution of compound 4 (70 mg, 0.4 mmol) in aqueous HCl acid (2 N, 2mL) were slowly added a NaNO₂ solution (41 mg, 0.6 mmol) in water (2 mL) and ethoxymethanedithioic acid potassium salt (96 mg, 0.6 mmol). The mixture was then stirred at room temperature overnight followed by dilution with water (10 mL) and EtOAc (20 mL). The layers were separated, and the aqueous layer was extracted with EtOAc (2×50 mL). The combined organic layers were washed with saturated aqueous NaHCO₃ solution (30 mL) and dried over Na₂SO₄. The Na₂SO₄ was removed by filtration, and the volatiles were removed under reduced pressure. The resulting residue was purified by flash chromatography using a mixture of hexane and ethyl acetate to provide the product 5 (30 mg, 26%) as yellow solid. LRMS (M+H⁺) m/z: cacld. 279.04, found 279.04; ¹H-NMR (CDCl₃): 9.01 (dd, 1H), 8.63 (dd, 1H), 7.93 (d, 1H), 7.44 (dd, 1H), 6.92 (d, 1H), 4.54 (q, 2H), 4.07 (s, 3H), 1.22 (t, 3H).

To a 0° C. solution of compound 5 (30 mg, 0.107 mmol) in EtOH (5 mL) was slowly added an aqueous KOH solution (0.5 mL, 1 N, 0.5 mmol). The mixture was then stirred at room temperature overnight followed by dilution with water (10 mL) and EtOAc (20 mL). The layers were separated, and the aqueous layer was extracted with EtOAc (2×50 mL). The combined organic layers were washed with saturated aqueous NaHCO₃ solution (30 mL) and dried over Na₂SO₄. The Na₂SO₄ was removed by filtration, and the volatiles were removed under reduced pressure. The resulting residue was purified by flash chromatography using a mixture of hexane and ethyl acetate to provide the product N (5 mg, 24%) as a yellow solid. LRMS (M+H⁺) m/z: cacld. 381.07, found 381.10; ¹H-NMR (CDCl₃): 9.00 (dd, 1H), 8.57 (dd, 1H), 7.83 (d, 1H), 7.46 (dd, 1H), 6.76 (d, 1H), 3.94 (s, 3H).

### Example 22

### Synthesis of Compound O

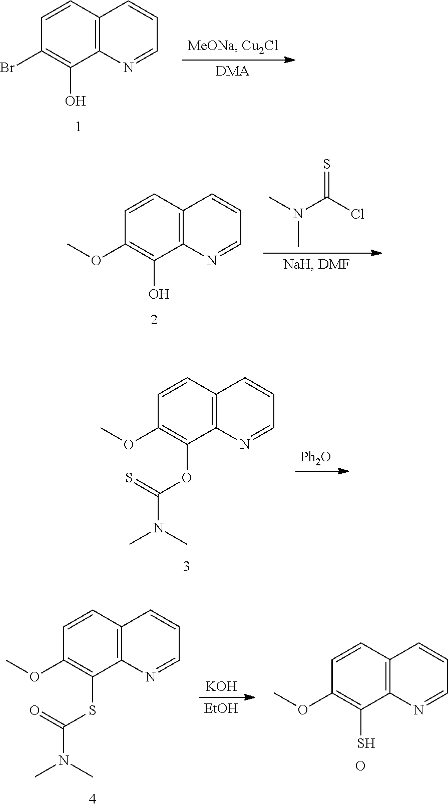

To a 0° C. solution of 7-bromoquinolin-8-ol (1.5 g, 6.7 mmol) in DMA (75 mL) were added NaOMe (3.6 g, 67 mmol) and CuCl₂ (270 mg, 2.0 mmol). The mixture was then stirred at 150° C., and then cooled down to room temperature followed by dilution with water (75 mL). Na₂EDTA (6 g) was added and the resulting mixture was stirred for another hour at room temperature followed by dilution with water (20 mL) and DCM (20 mL). The layers were separated, and the aqueous layer was extracted with DCM (3×50 mL). The combined organic layers were washed with brine (30 mL) and dried over Na₂SO₄. The Na₂SO₄ was removed by filtration, and the volatiles were removed under reduced pressure. The resulting residue was purified by flash chromatography using a mixture of hexane and ethyl acetate to provide the product 2 (0.7 g, 60%) as a yellow solid. ¹H-NMR (CDCl₃): 8.76 (dd, 1H), 8.09 (dd, 1H), 7.35-7.26 (m, 3H), 4.06 (s, 1H).

To a 0° C. solution of compound 2 (400 mg, 22.5 mmol) in DMF (20 mL) was added NaH (137 mg, 34.3 mmol). The reaction mixture was stirred at the same temperature for 1 hour and then (dimethylamino)methanethioyl chloride (424 mg, 34.3 mmol) was added. The resulting reaction mixture was stirred at 80° C. overnight. It's then cooled down to room temperature and quenched with water (40 mL) and EtOAc (40 mL). The layers were separated, and the aqueous layer was extracted with EtOAc (3×50 mL). The combined organic layers were washed with brine (30 mL) and dried over Na₂SO₄. The Na₂SO₄ was removed by filtration, and the volatiles were removed under reduced pressure. The resulting residue was purified by flash chromatography using a mixture of hexane and ethyl acetate to provide the product 3 (190 mg, 32%). ¹H-NMR (CDCl₃): 8.92 (dd, 1H), 8.10 (dd, 1H), 7.74 (d, 1H), 7.42 (d, 1H), 7.29 (t, 1H), 4.02 (s, 3H), 3.54 (s, 3H), 3.52 (s, 3H).

A solution of compound 3 (200 mg, 0.76 mmol) in diphenyl ether (20 mL) was stirred at 250° C. for 2 hours. It's then cooled down to room temperature und purified by flash chromatography using a mixture of hexane and ethyl acetate to obtain compound 4 (43 mg, 22%) as a yellow solid. ¹H-NMR (CDCl₃): 9.00 (dd, 1H), 8.11 (dd, 1H), 7.93 (d, 1H), 7.42 (d, 1H), 7.31 (t, 1H), 4.06 (s, 3H), 3.29 (br, 3H), 3.00 (br, 3H).

To a 0° C. solution of compound 4 (40 mg, 0.15 mmol) in THF/MeOH (2 mL/1 mL) was slowly added an aqueous KOH solution (0.6 mL, 1 N, 0.6 mmol). The mixture was then stirred at 55° C. overnight. And it was cooled down to room temperature and neutralized with AcOH followed by dilution with water (10 mL) and EtOAc (20 mL). The layers were separated, and the aqueous layer was extracted with EtOAc (2×50 mL). The combined organic layers were washed with saturated aqueous NaHCO₃ solution (30 mL) and dried over Na₂SO₄. The Na₂SO₄ was removed by filtration, and the volatiles were removed under reduced pressure. The resulting residue was purified by preparative HPLC to afford target compound O (8 mg, 28%) as a yellow solid. LRMS (M+H⁺) m/z: cacld. 381.1, found 381.1; ¹H-NMR (CDCl₃): 8.91 (dd, 1H), 8.10 (dd, 1H), 7.58 (d, 1H), 7.33-7.28 (m, 2H), 5.53 (br, 1H), 4.07 (s, 3H).

### Table of Compounds

The structures of the compounds prepared and assayed for Rpn11 and Csn5 inhibition are listed on Tables 5, 6A and 6B. All compounds on Table 6B demonstrated IC₅₀ inhibitory activity in the Rpn11 assay that was sufficient to indicate that these compounds would display reasonable inhibitory activity in vivo.

### BIOLOGICAL EXAMPLES

### Bioexample 1

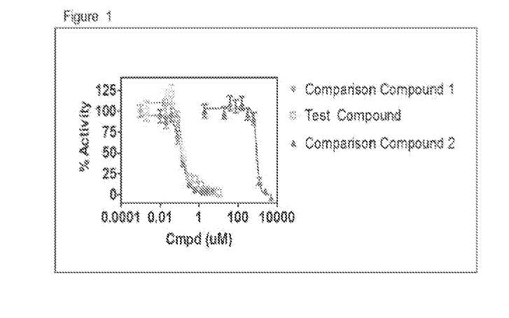

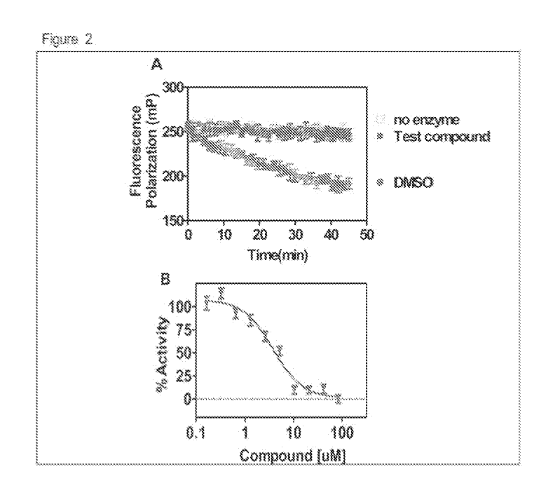

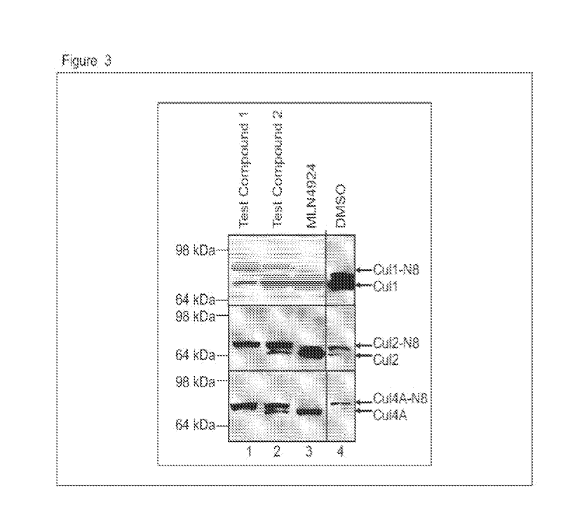

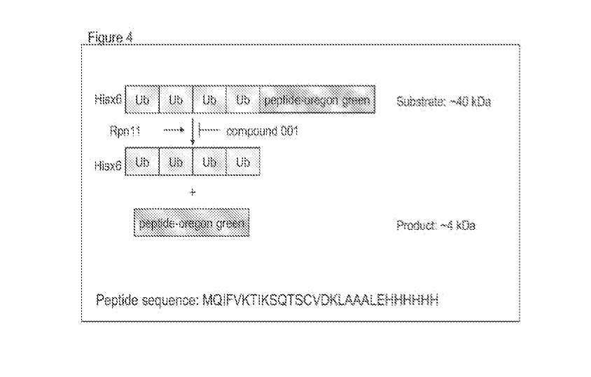

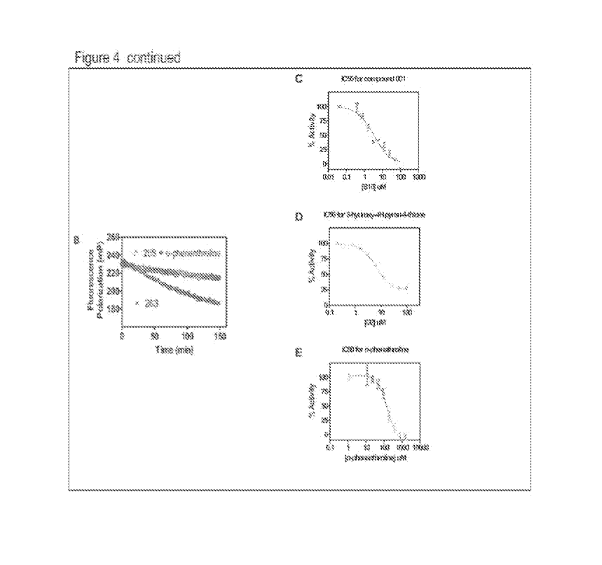

As shown in FIG. 1, HeLa cells engineered to stably express the proteasome reporter Ub^{G76V}-GFP were incubated with Comparison Compound 1 (MG132; N-(benzyloxycarbonyl)leucinylleucinylleucinal (Z-Leu-Leu-Leu-al); CAS Reg. No. 133407-82-6) for 2 hours to accumulate the reporter. The cells were then washed to remove Comparison Compound 1 and resuspended in fresh medium containing cycloheximide (to block further protein synthesis) and test agent (Comparison Compound 1, Compound 1, or Comparison Compound 2 (O-PT; o-phenanthroline). After 90 min at 37° C., the level of remaining Ub^{G76V}-GFP was measured by quantitative fluorescence microscopy. Compound 1 performed similar to MG132 (Comparison Compound 1). Compound 1 outperformed another Rpn11 inhibitor, o-phenanthroline (Comparison Compound 2). IC50 values and 95% confidence intervals (in parenthesis) are as follows: Compound 1:0.12 μM (0.1˜0.15 μM); MG132: 0.23 μM (0.19˜0.28 μM) and o-phenanthroline (O-PT): 590 μM (560˜620 μM).

### Bioexample 2

As shown in FIG. 2(A) Cu11-Nedd8OG was mixed with purified CSN and fluorescence polarization was monitored in a low volume plate using the Analyst AD fluorimeter. Release of Nedd8OG from the Cu11-Nedd8OG conjugate by CSN causes a decrease in fluorescence polarization which can be inhibited with Compound 1 (test compound). In FIG. 2(B), the reaction was performed with a dose escalation of Compound 1 to determine IC50. IC50 values and 95% confidence intervals (in parenthesis) are as follows: Compound 1: 3.8 μM (2.2˜6.3 μM).

### Bioexample 3

As shown in FIG. 3, this BIOEXAMPLE tested accumulation of neddylated forms of Cullins 1, 2, and 4A in tHEK-293T cells treated with various compounds. Test compounds include Compound 1 (“Test Compound 1”), Compound 101 (“Test Compound 2”), and MLN4924 (“Comparison Compound”; Sulfamic acid, [(1S,2S,4R)-4-[4-[[(1S)-2,3-dihydro-1H-inden-1-yl]amino]-7H-pyrrolo[2,3-d]pyrimidin-7-yl]-2-hydroxycyclopentyl]methyl ester; CAS Reg. No. 905579-51-3). Human HEK-293T cells were treated with 100 μM compound 1, 100 μM compound 101, 3 μM MLN4924 or 1% DMSO for 15 min. at 37° C. At the end of the incubation, cells were harvested and lysed. All cell lysates were fractionated by SDS-PAGE, proteins were transferred to nitrocellulose, and Cu11, Cu12 and Cu14 was detected by immunoblotting with an antibody that recognizes human Cu11, Cu12 or Cu14. Cu11-N8: human Cu11 modified with Nedd8. Cu12-N8: human Cu12 modified with Nedd8. Cu14-N8: human Cu14 modified with Nedd8. The experiment shows is that in the presence of 100 μM compound 001, Cu11, Cu12 and Cu14 was converted to a species that was largely modified with Nedd8. Inactive compound 1 analog, compound 101 does not induce full conversion of Cu11, Cu12 nor Cu14 to a species modified by Nedd8. Finally, neddylation inhibitor MLN4924 has the opposite effect, namely Cu11, Cu12 and Cu14 remain in the non-neddylated form.

### Bioexample 4

As seens in FIG. 4A) the Rpn11 substrate consisted of six histidine residues followed by four ubiquitin sequences fallowed by the peptide sequence MQIFVKTIKSQTSCVDKLAAALEHHHHHH. The Oregon Green fluorophore was attached to the cysteine residue via maleimide chemistry. Following cleavage of the substrate by Rpn11, the product MQIFVKTIKSQTSCVDKLAAALEHHHHHH coupled to Oregon Green was released. The change in molecular weight of Oregon Green containing protein was detected by fluorescence polarization. The Rpn11 reaction can he inhibited with compound 1. In FIG. 4(B) progress curves for product generation by 26S proteasome (which contains Rpn11). Rpn11 cleaves the substrate immediately after the fourth ubiquitin (Ub). 5 nM Ub-Ub-Ub-Ub-peptide-oregon green substrate was incubated with 0.5 nM purified 26S proteasome for 120 min at room temperature. As labeled peptide is liberated, the fluorescence polarization diminishes. The cleavage reaction can be inhibited by the o-phenanthroline. In FIG. 4(C-E) dose-response curve for inhibition of Rpn11 by compound 1 (C), 3-hydroxy-4H-pyran-4-thione (D) or o-phenanthroline (E) were generated by adding varying doses of compound to the reaction mixture. At 120 minutes, fluorescence polarization was measured. Percent activity was calculated based on the amount of enzyme activity observed in the absence of added test agent. IC₅₀ values and 95% confidence intervals (in parenthesis) are as follows: COMPOUND 1: 3.3 μM (2.2˜6 μM); 3-hydroxy-4H-pyran-4-thione: 0.6 μM (4.7˜7.5 μM) and o-phenanthroline: 158 μM (116˜185 μM).

### Bioexample 5

As seen in FIG. 5, tested compounds inhibit Rpn11 activity in vitro. BIOEXAMPLE 4 is repeated with compounds as described herein.

### Bioexample 6

As seen in FIG. 6, biochemical assays were performed as described in BIOEXAMPLEs 2 and 4. The cell-based anti-tumor assay was performed by treating HCT-116 colon cancer cells, C148 and Hep3B liver cells with a dose titration of test compound in standard cell culture media continuously for 48 hours. At the 48 hour time point, cell viability was measured using Cell-Titer Glo (Promega) according to manufacturer's recommended protocol. The list of compound IDs, structures and corresponding IC₅₀ values in biochemical Rpn11 and Csn5 assays and a cell-based tumor cell growth assay are shown.

### Bioexample 7

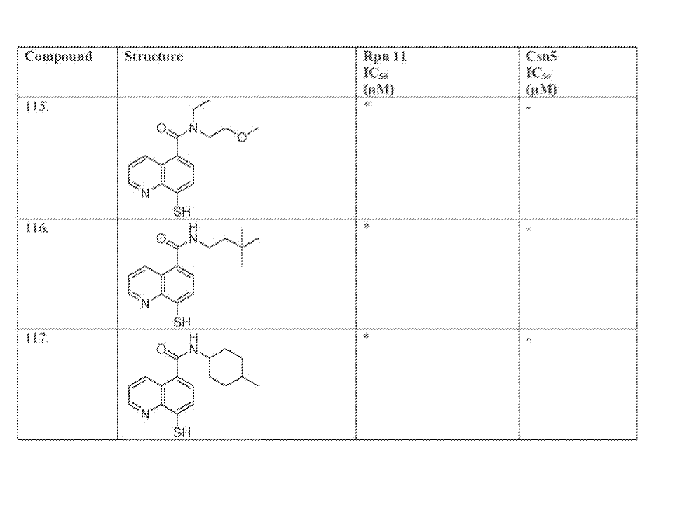

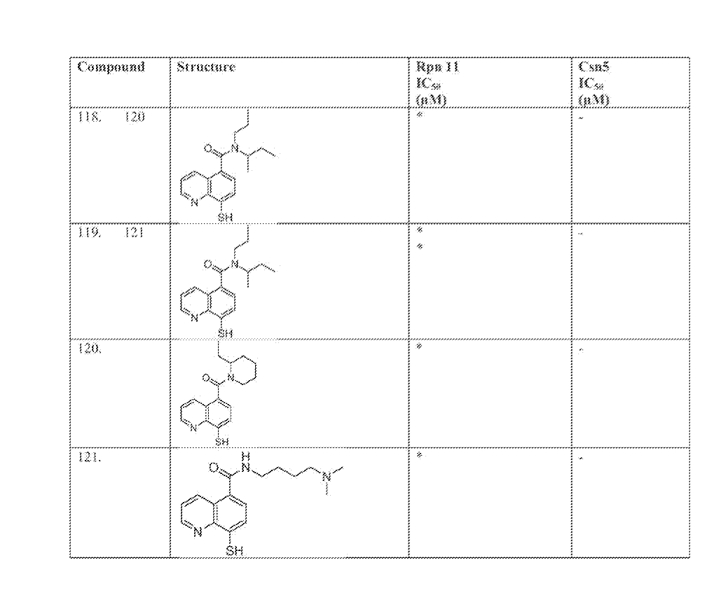

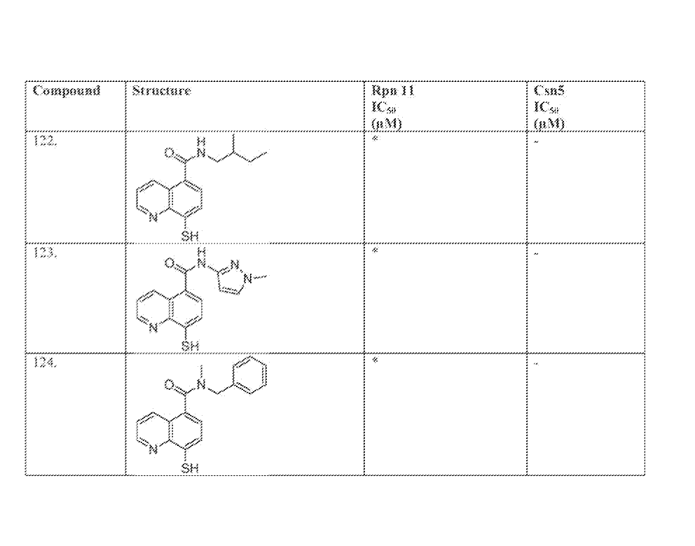

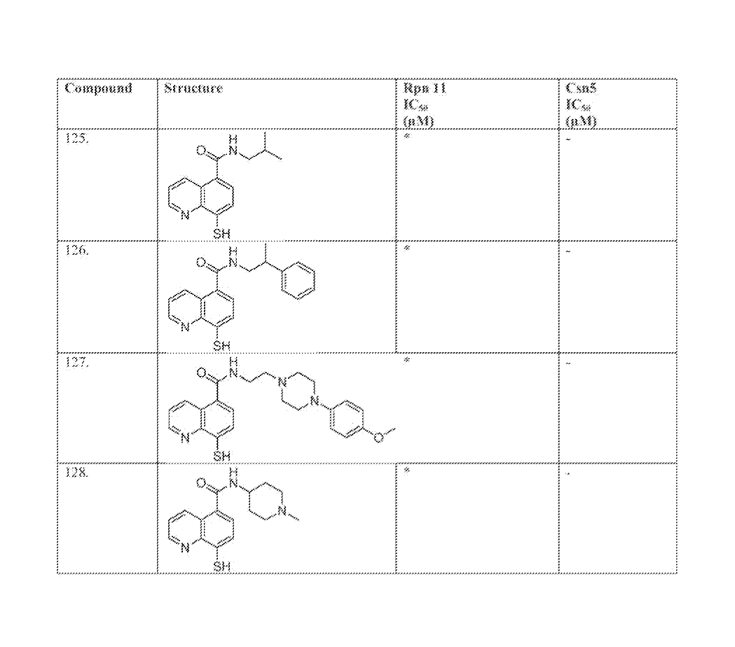

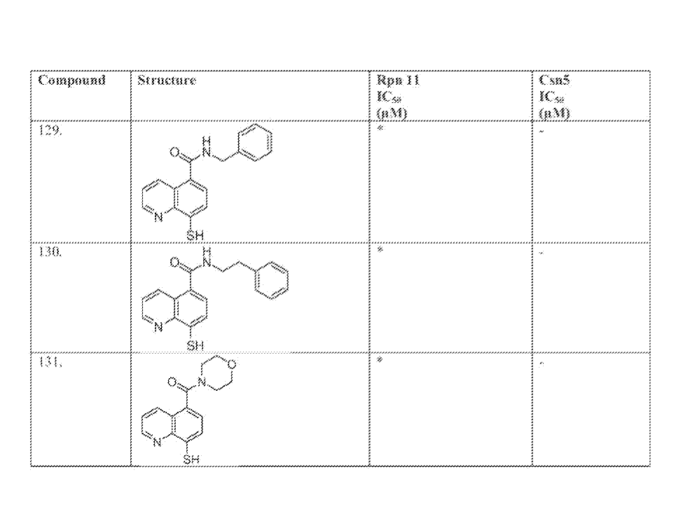

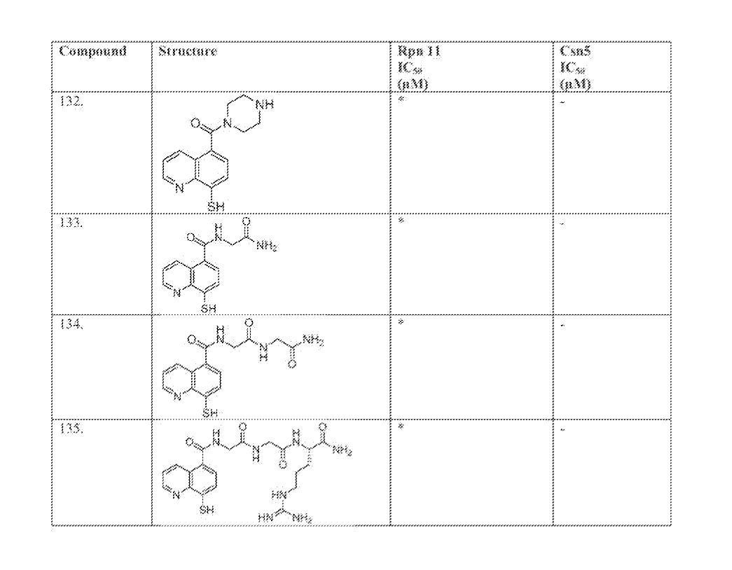

As shown in FIG. 7, an IC₅₀ was determined for compound 001 in the Rpn11 Assay. An 8-point compound 001 dose titration curve was performed, by incubating Rpn11-containing 26S proteasome, Rpn11 substrate and compound (dose range 50 μM to 0.1 μM) and monitoring fluorescence polarization as per BIOEXAMPLE 4. Remaining activity as compared to DMSO control (% activity) was calculated and plotted versus compound 1 concentration and the curve was fit to a four-parameter fit model to obtain an IC₅₀ value.

### Bioexample 8

As shown in FIG. 8, an orthogonal method for monitoring Rpn11 reaction was conducted. Rpn11 reactions were conducted as described in BIOEXAMPLE 4. At the 1 hr time point, reactions were boiled in SDS-PAGE sample buffer and loaded onto SDS-PAGE gel. Following SDS-PAGE electrophoresis, the gel was scanned using a Typhoon Gel scanner to detect Oregon Green fluorescence (lower panel). The proteins on the gel were subsequently transferred to nitrocellulose and ubiquitin was revealed by use of an anti-ubiquitin antibody coupled to horse radish peroxidase followed by chemiluminiscence detection.

### Bioexample 9

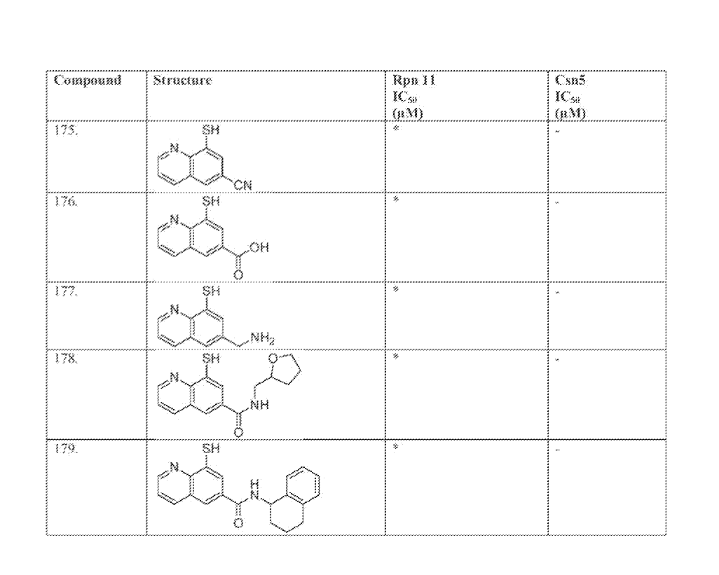

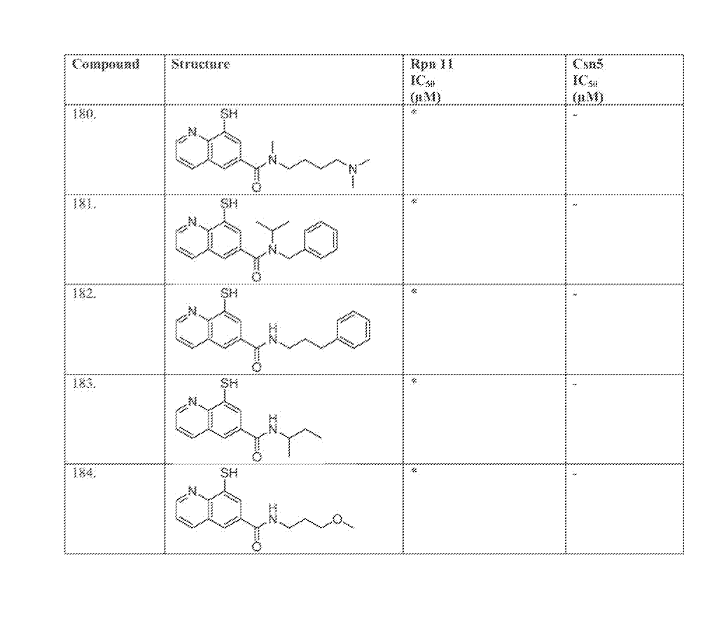

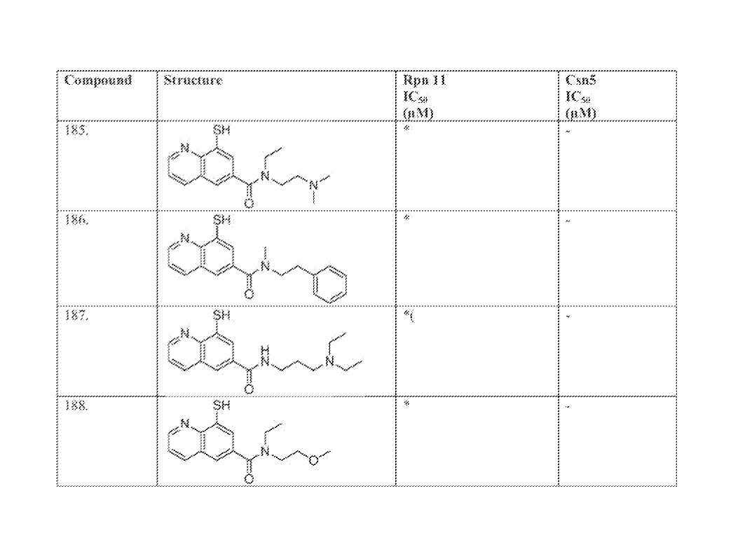

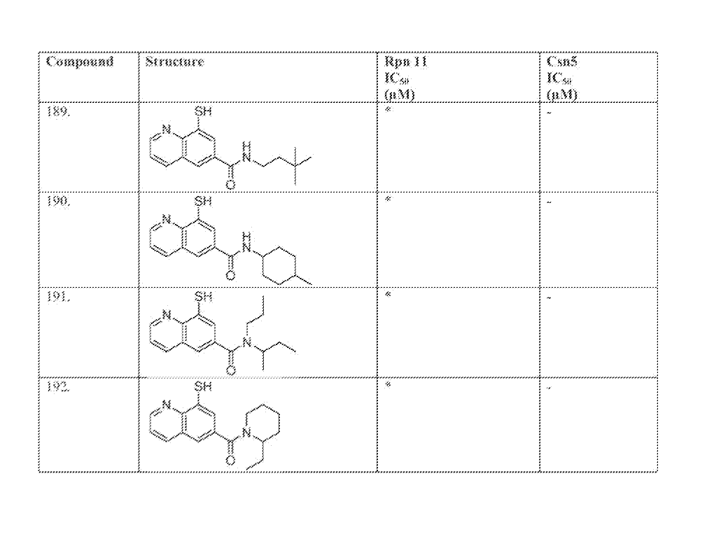

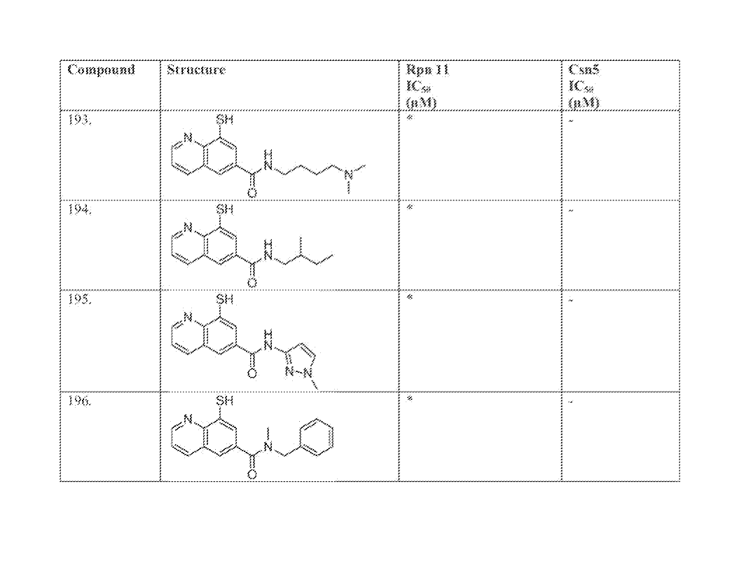

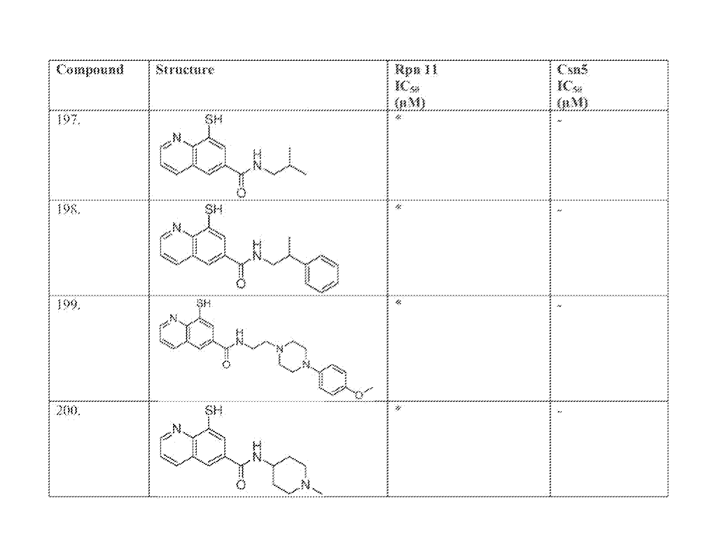

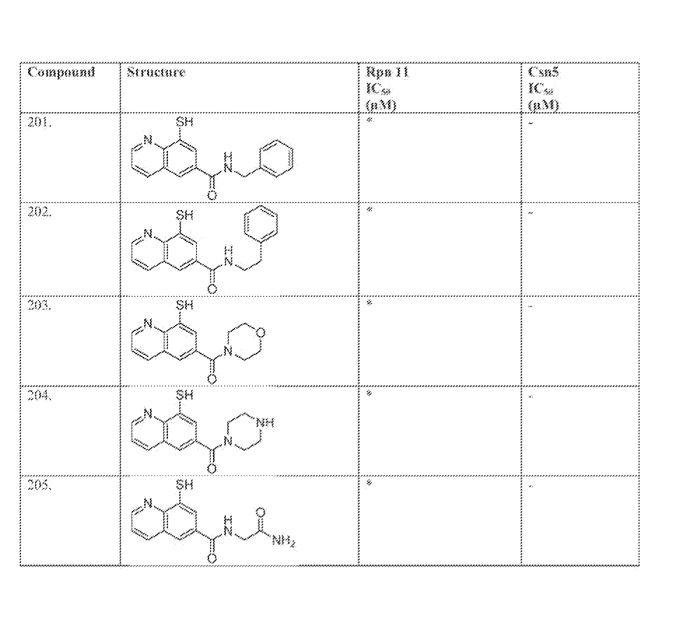

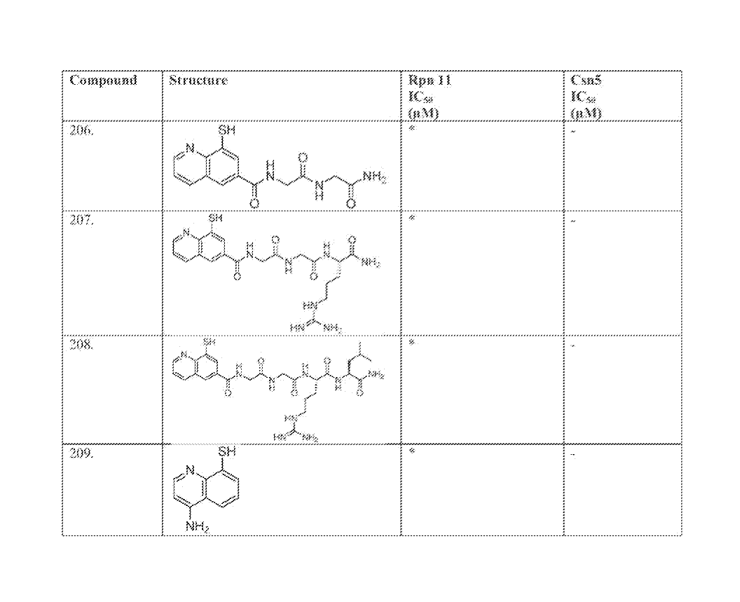

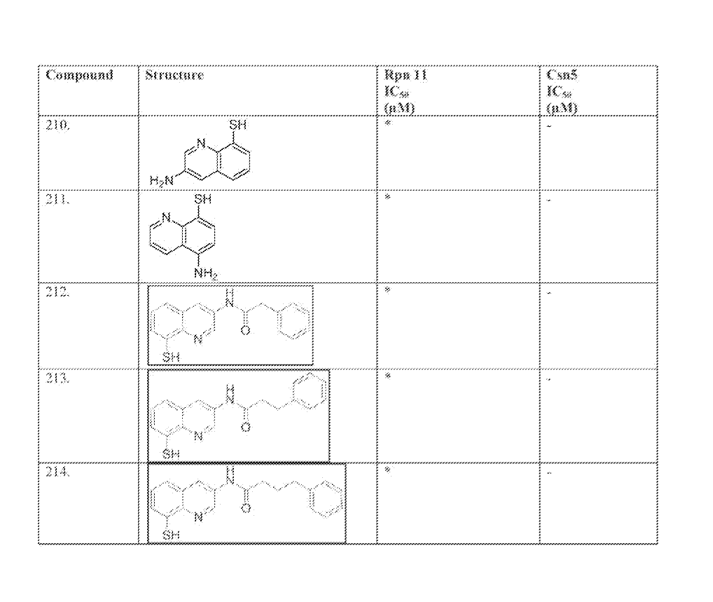

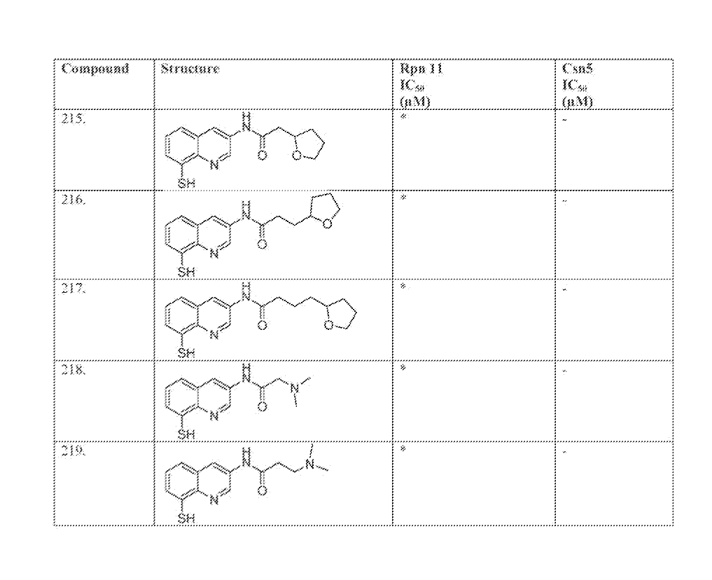

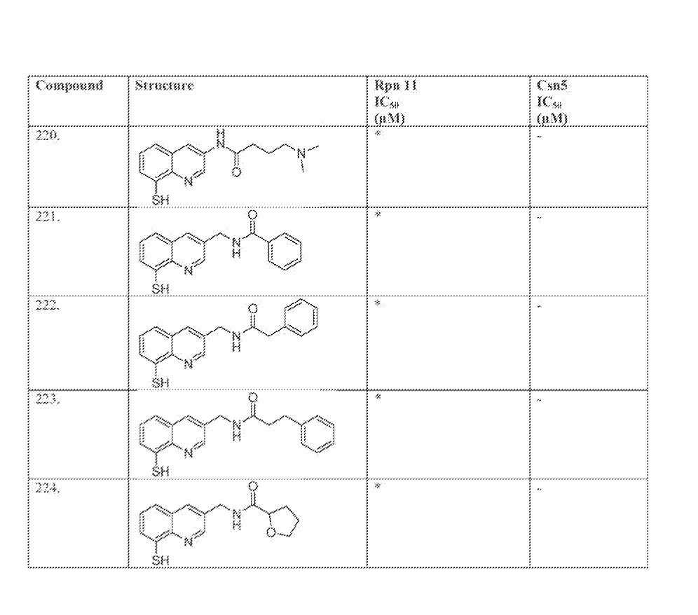

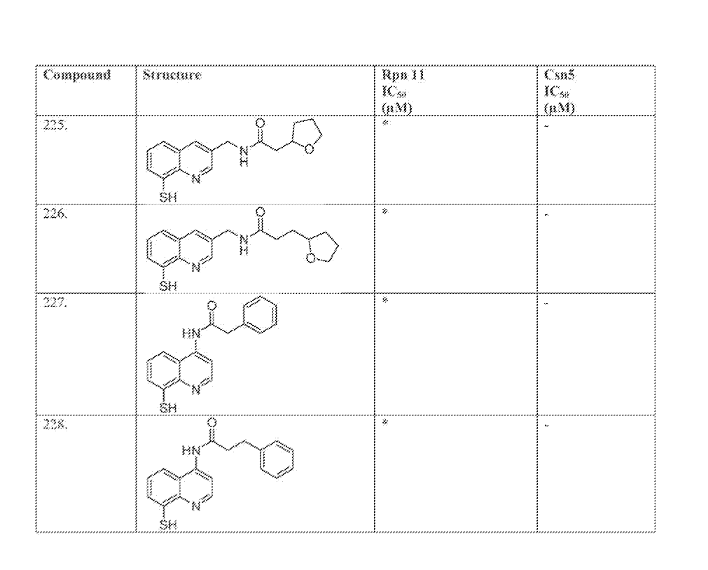

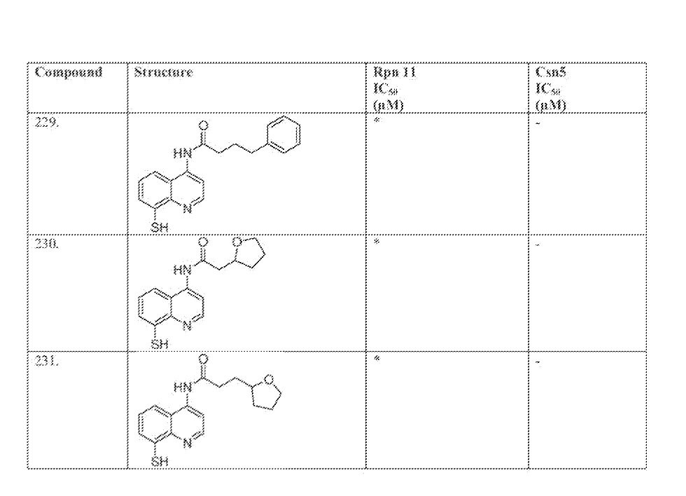

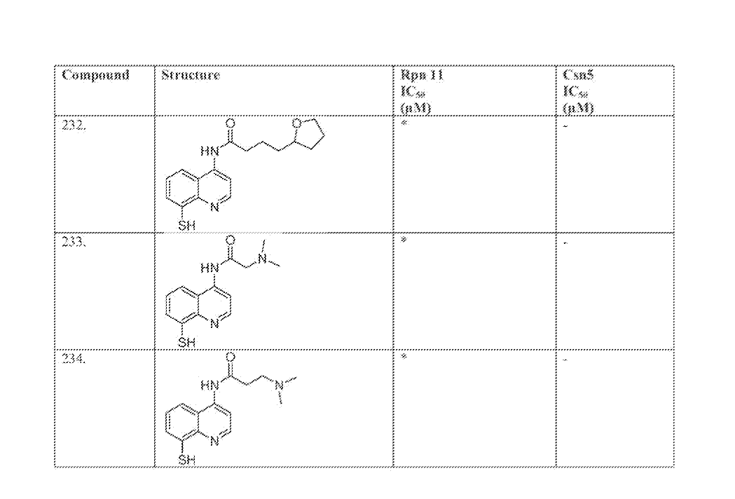

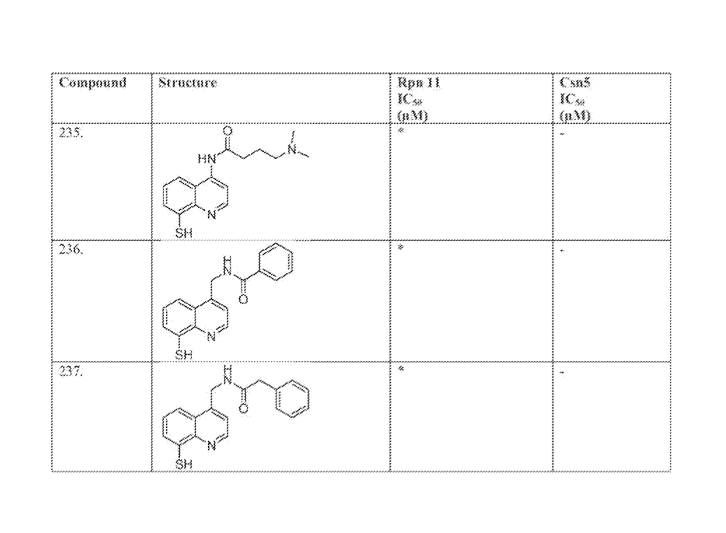

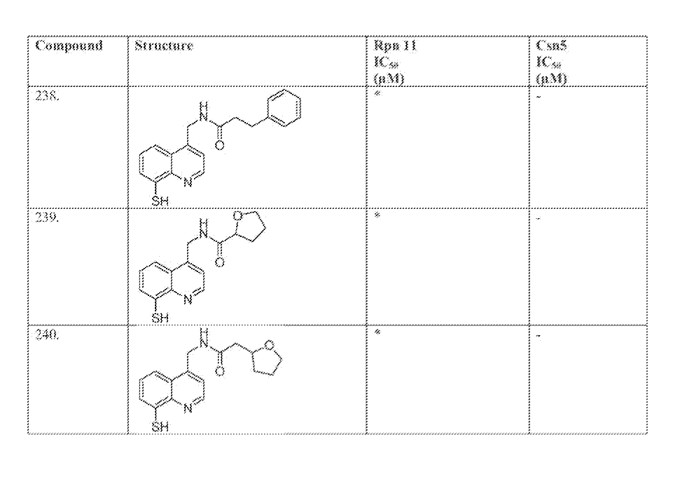

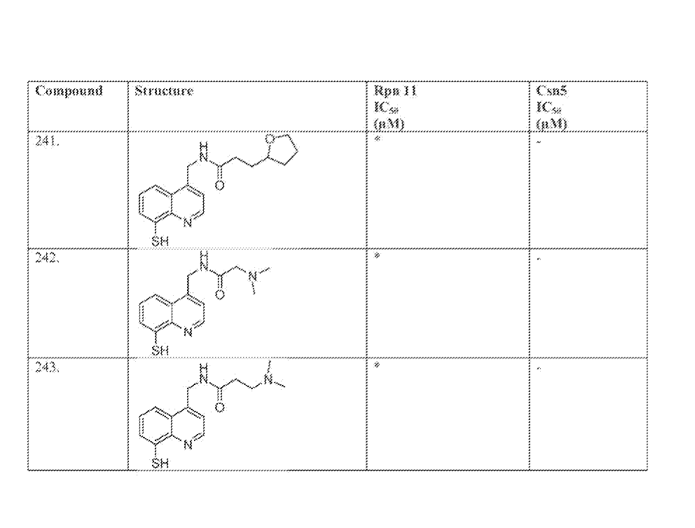

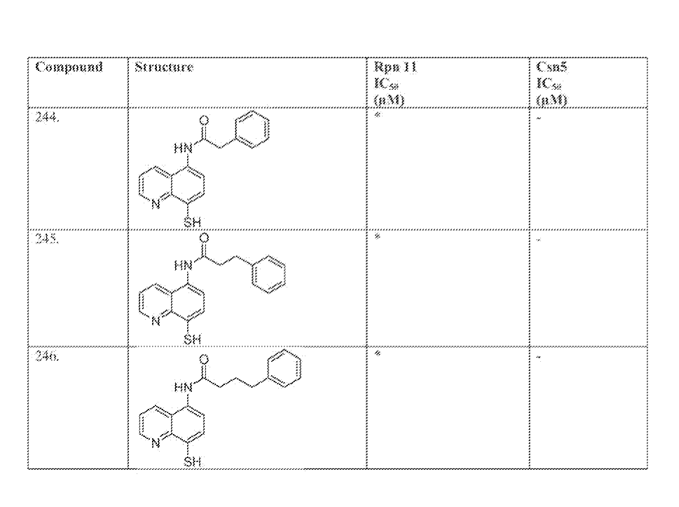

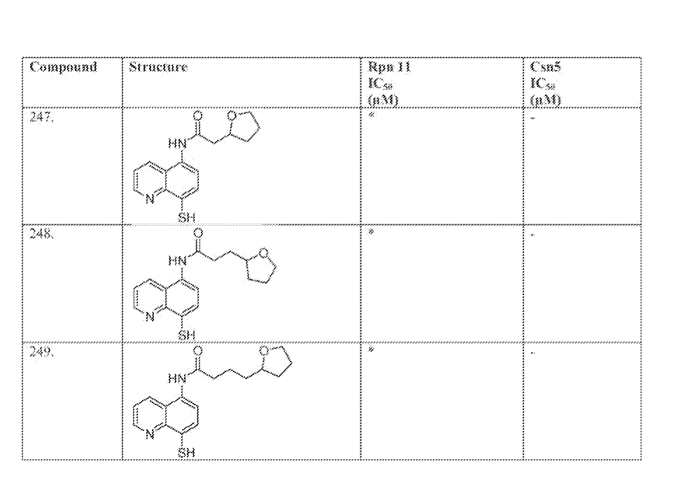

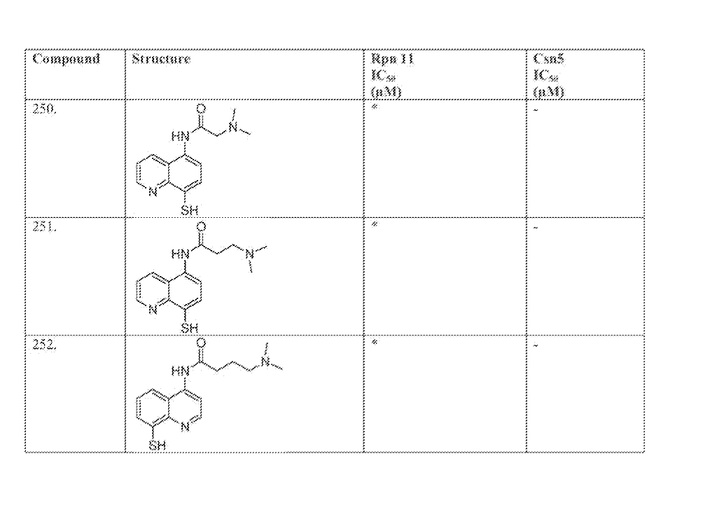

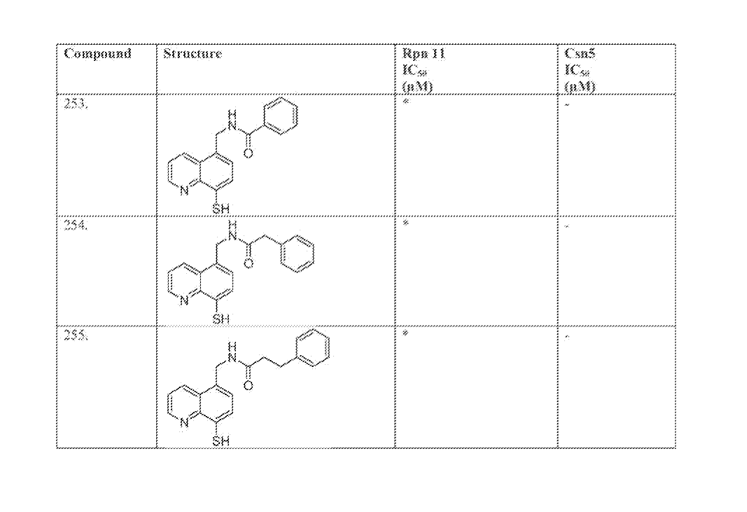

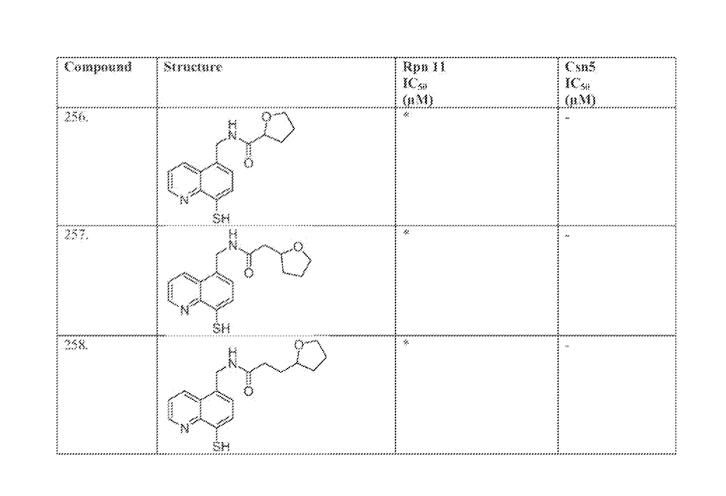

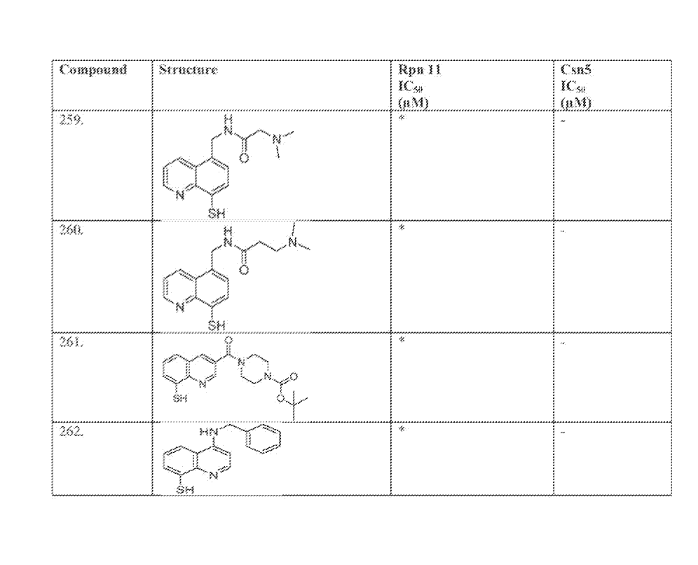

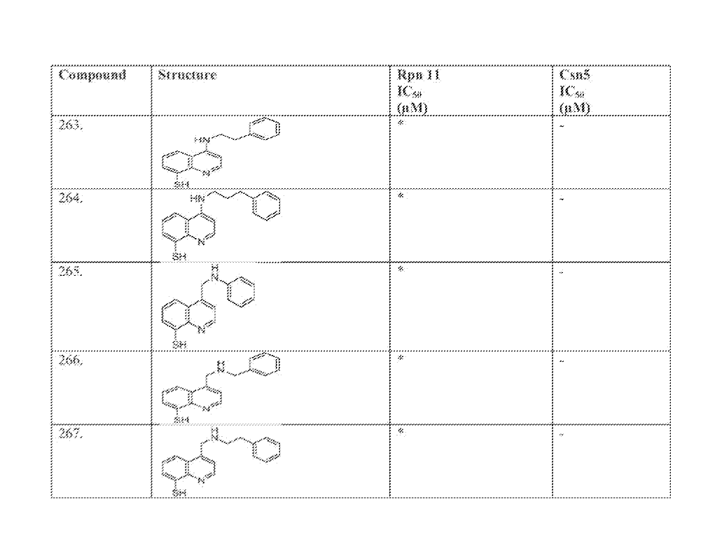

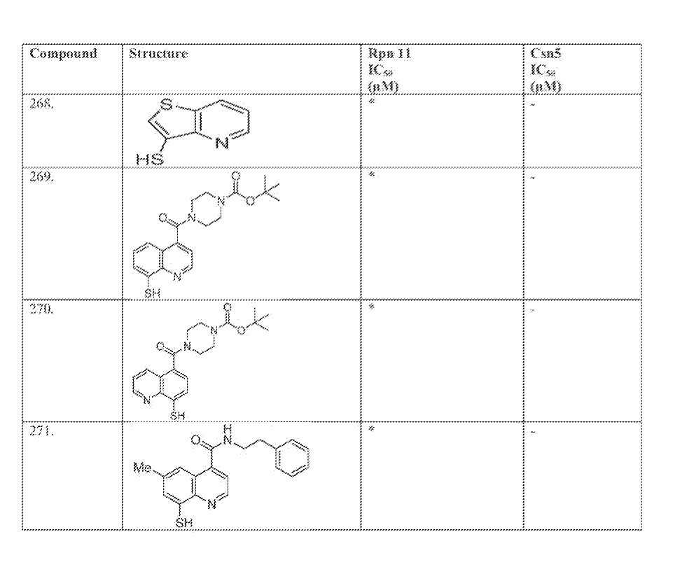

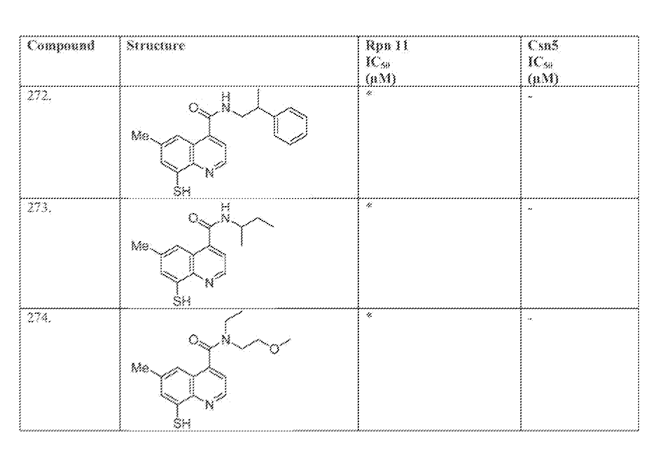

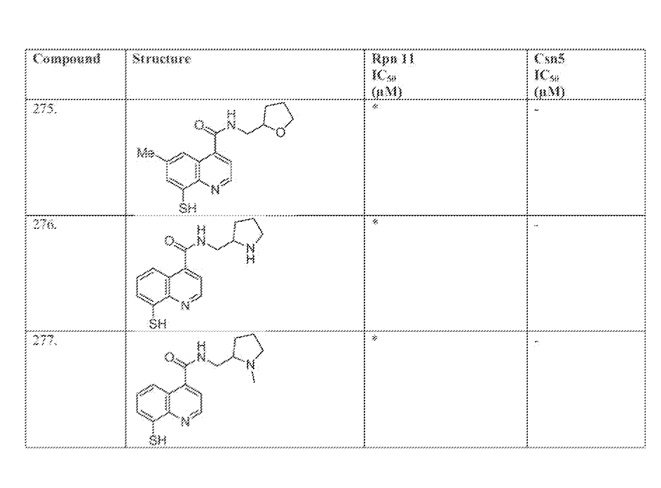

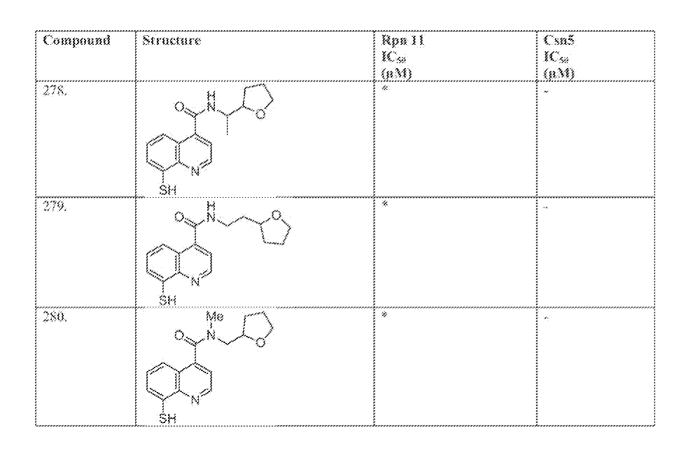

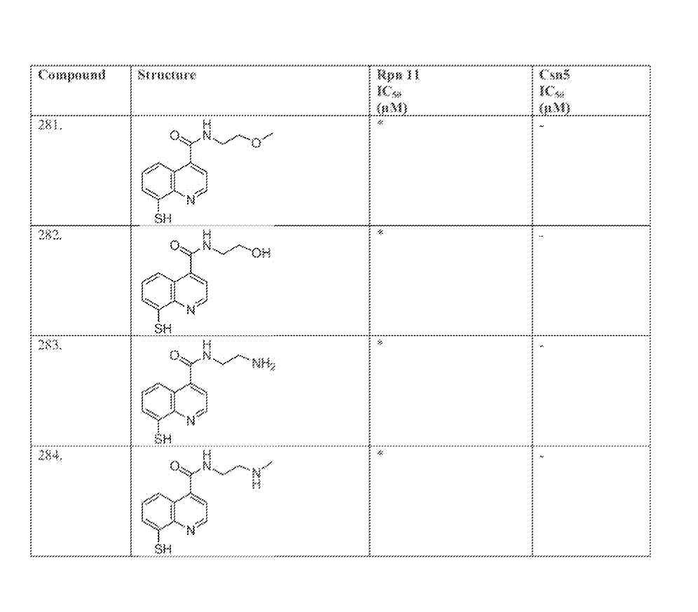

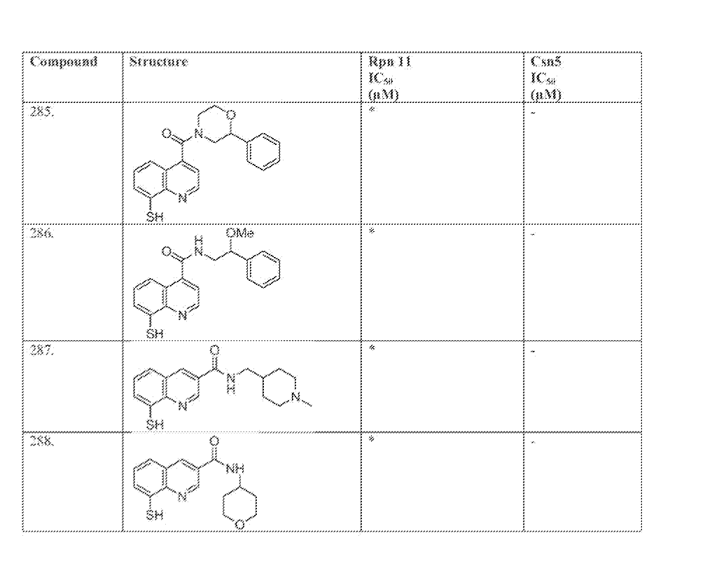

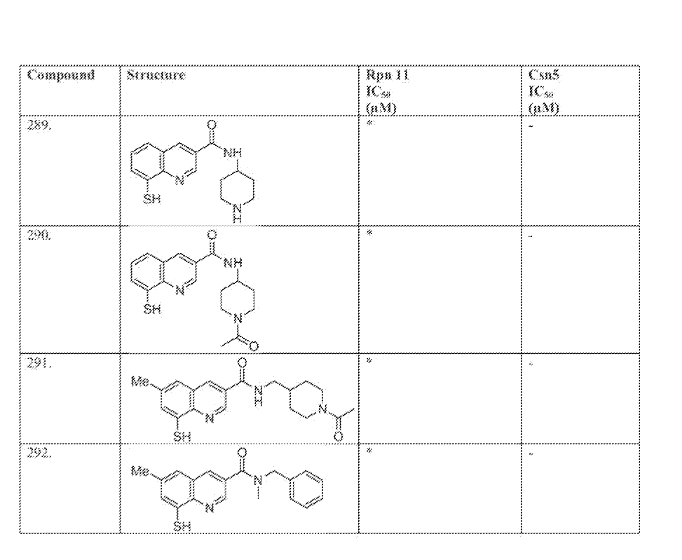

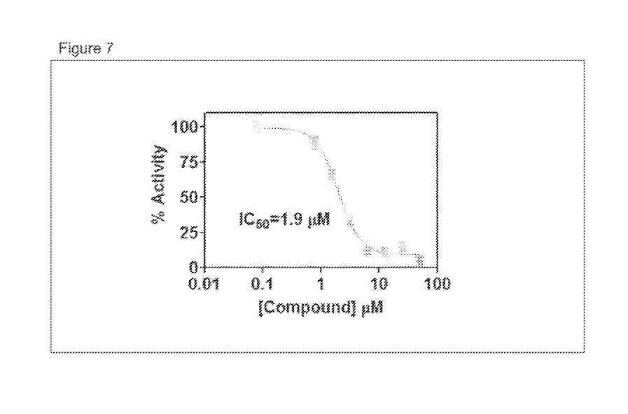

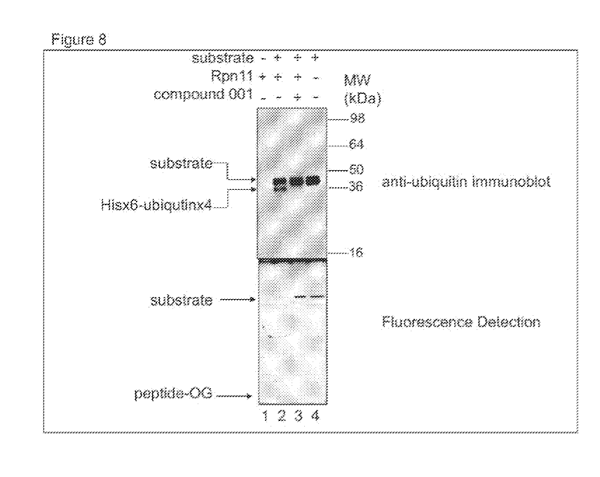

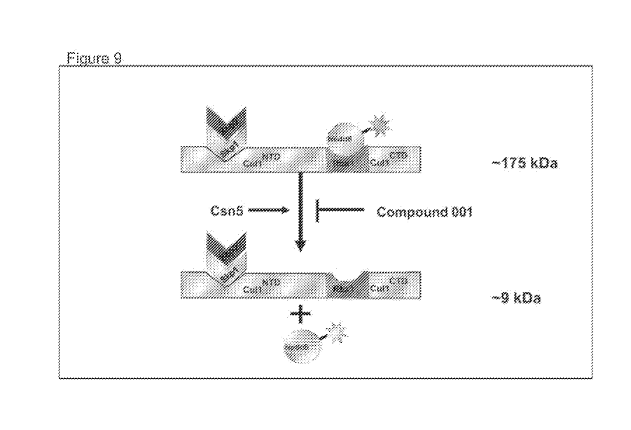

As shown in FIG. 9, THE Csn5 assay is drawn as a schematic representation. Csn5 cleaves Nedd8-modified Oregon Green (Nedd8OG) from the SCF^{Skp}2 complex composed of Cu11^{NTD}, Cu11^{CTD}, Rbx1, Skp1 and Skp2. Prior to cleavage by Csn5, the fluorophore Oregon Green is present in a ˜175 kDa complex. Post-cleavage, the fluorophore is found in a ˜9 kDa complex. The Oregon Green fluorophore is attached to an N-terminal cysteine residue in Nedd8 via maleimide chemistry. The change in molecular weight of Oregon Green containing protein can be detected by fluorescence polarization. The change in molecular weight of Oregon Green containing protein can be detected by fluorescence polarization. The Csn5 reaction can be inhibited with compound 001.

### Bioexample 10

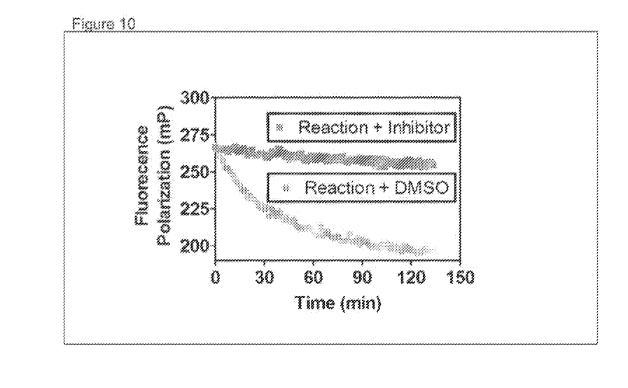

As shown in FIG. 10, progress curves for the Csn5 reaction. Substrate (4.5 nM), Cop9 signalosome (0.033 nM) and DMSO (1%) were mixed in a microplate and fluorescence polarization was monitored using a fluorescence plate reader (Molecular Devices: Analyst AD). DMSO can be substituted with compound 001 (100 μM) [‘inhibitor’]. Buffer was comprised of 25 mM Tris pH 7.6, 50 mM NaCl, 1% glycerol, 1 mM DTT, 0.01% TX-100, 15 μg/ml ovalbumin, 25 mM trehalose).

### Bioexample 11

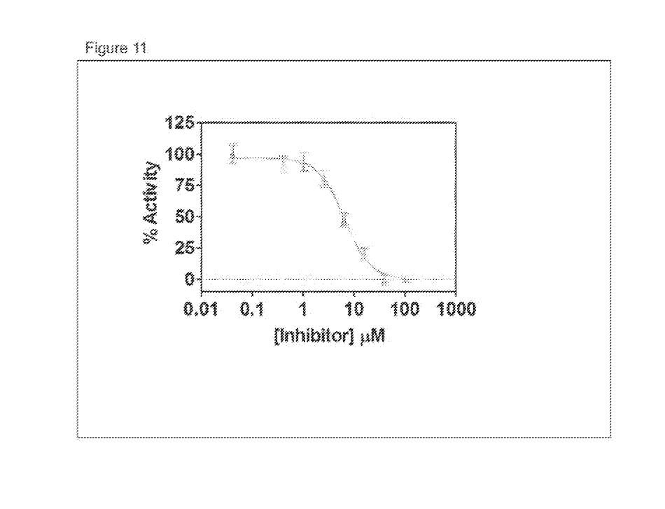

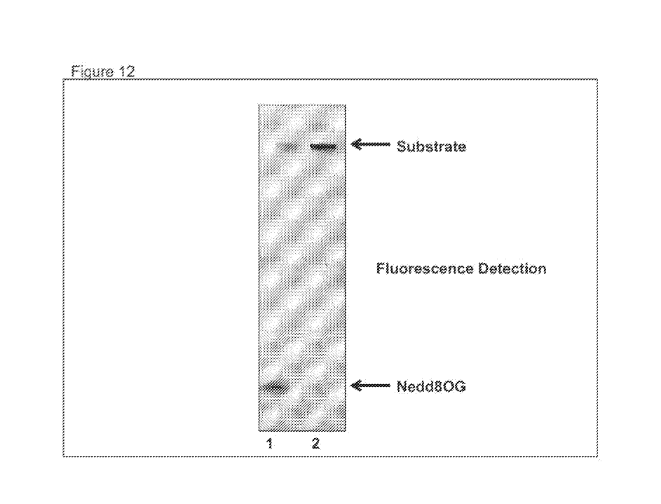

As shown in FIG. 11, IC₅₀ determination for compound 001 on Csn5. An 8-point compound 001 dose titration curve was performed by incubating Cop9 signalsome, Csn5 substrate and compound (dose range 100 μM to 0.05 μM) and monitoring fluorescence polarization as per BIOEXAMPLE 10. The remaining activity as compared to DMSO control (% activity) was calculated and plotted versus compound 001 concentration and the curve was fit to a four-parameter fit model to obtain an IC₅₀ value of 6.5 μM with a 95% confidence interval of 5.3 to 7.9 μM.

### Bioexample 12

As shown in Example 12, an orthogonal method for detecting Csn5 activity. Csn5 reactions were conducted as per BIOEXAMPLE 10. At the 1 hr time point, reactions were boiled in SDS-PAGE sample buffer and loaded onto SDS-PAGE gel. Following SDS-PAGE electrophoresis, the gel was scanned using a Typhoon Gel scanner to detect Oregon Green fluorescence. The presence of substrate and enzyme (Csn5) leads to detection of product Nedd8OG by fluorescence (lane 1). Inclusion of 100 μM compound (lane 2) leads to no product formation.

### Bioexample 13

As shown in FIG. 13, a cell-based Csn5 Assay. Because cullins undergo a dynamic cycling between neddylated and unneddylated forms by the action of NAE1 and Csn5, respectively, a Csn5 inhibitor will block the removal of Nedd8 from cullin resulting in the accumulation of Nedd8-cullin. HEK-293T cells were treated with compound 001 for 30 min., washed with PBS and lysed in gel loading buffer. Clarified cell lysates (40 μg) were electrophoresed, transferred to nitrocellulose and immunoblotted with α-Cu11 or α-actin sera. The two forms can be easily distinguished by immunoblotting with anti-Cu11 as shown Under steady state conditions, the majority of Cu11 exists in the unneddylated form (lane 7) and treatment of cells with increasing compound 001 concentration shifts the Cu11 population from the undeddylated to the neddylated form (lanes 1-6).

### Bioexample 14

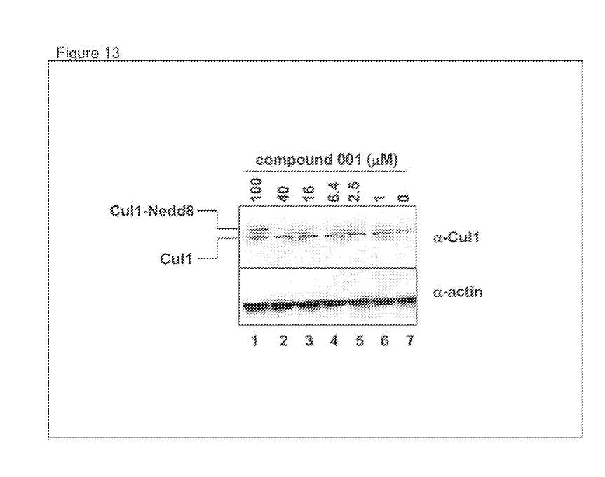

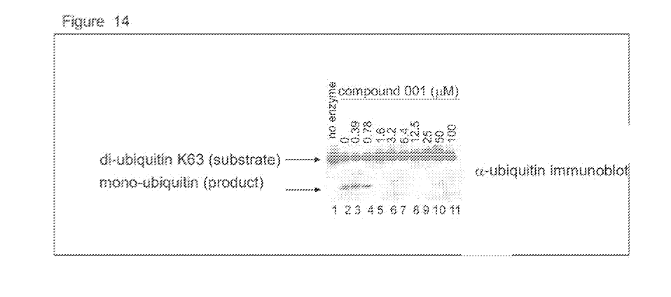

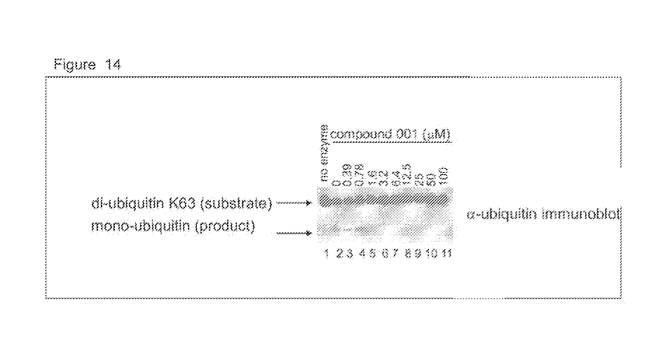

As shown in FIG. 14, an AMSH-LP assay. AMSH-LP (50 nM), di-ubiquitin K63 (1 μM) and DMSO (1%) OR compound 001 (dose range: 0.39 μM to 100 μM) were incubated in buffer (100 mM Tris pH 7.6, 25 mM NaCl, 5 mM MgCl₂, 1 for 20 hours at room temperature and subsequently samples were boiled in loading buffer and separated by SDS-PAGE electrophoresis. Proteins were transferred to nitrocellulose and blotted with anti-ubiquitin antibodies followed by secondary antibodies coupled to horseradish peroxidase. Bands were revealed by, AMSH-LP (enzyme) and di-ubiquitin-K63 (substrate) incubation leads to the formation of mono-ubiquitin product (lane 2). When substrate and enzyme are incubated with increasing concentrations of compound 001 (lanes 3-11), product formation is inhibited with an approximate IC₅₀ of 1.6 μM. Formation of product is dependant on the presence of AMSH-LP (lane 1).

### Bioexample 15

### Diagnostic Use

Patient's cells are obtained and grown in culture. The cells are treated or not with a compound as described herein for an incubation period at about 37° C. At the end of the incubation, the cells are harvested and lysed. The cell lysates are fractionated by SDS-PAGE, the proteins are transferred to nitrocellulose, and Cu14 is detected by immunoblotting with an antibody that recognizes human Cu14. The compound as described herein is found effective in preventing the dissociation of Nedd8 from Cu14 by analyzing the immunoblot. A patient with cells that respond to treatment with a compound as described herein in such a diagnostic assay is determined to be a candidate for further treatment with the compound. The diagnostic assay as described herein provides the advantage of increasing the likelihood that a patient will respond to therapy and allows potential non-responders to be treated with alternative therapies more rapidly.

### Bioexample 16

### Inhibitory Characteristics of Selected Compounds

Using the techniques described in the preceding BIOEXAMPLEs the inhibitory characteristics of several selected compounds have been determined. The following able shows the IC50 for several compounds for both RPN11 and CSN5. IC50 values may vary depending on the conditions used for testing.

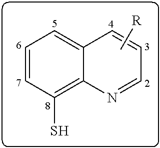

| | | RPN11 | CSN5 |
|---|---|---|---|
| Compound | R | IC50 (uM) | IC50 (uM) |
| 1 | H | 1.1 | 3.8 |
| 3 | 2-Me | 6.1 | N.D. |
| 4 | 3-Me | 1.3 | 7.7 |
| 5 | 4-Me | 2.5 | 22.2 |
| 6 | 5-Me | 1.1 | 4.1 |
| 7 | 6-Me | 0.8 | 2.1 |
| 8 | 7-Me | ND. | N.D. |

### BIBLIOGRAPHY

All of the following references are incorporated herein by reference as if fully repeated.

Adler, A. S., M. Lin, et al. (2006), “Genetic regulators of large-scale transcriptional signatures in cancer.” Nat Genet 38 (4); 421-30.

Adler, A. S., L. E. Littlepage, et al. (2008). “CSN5 isopeptidase activity links COP9 signalosome activation to breast cancer progression.” Cancer Res 68(2); 506-15.

Cope, G. A. and R. J. Deshaies (2003). “COP9 signalosome: a multifunctional regulator of SCF and other cullin-based ubiquitin ligases.” Cell 114 (6): 663-71.

Cope, G. A., G. S. Suh, et al. (2002). “Rule of predicted metalloprotease motif of Jab1/Csn5 in cleavage of Nedd8 from Cu11,” Science 298 (5593): 608-11.

Deshaies, R. J., E. D. Emberley, et al. (2010) Control of cullin-RING ubiquitin ligase activity by Nedd8. Conjugation and Deconjugation of Ubiquitin Family Modifiers. M. Groettrup, Landes Bioscience; 41-56.

Gallery, M., J. L. Blank, et al. (2007). “The JAMM motif of human deubiquitinase Poh1 is essential for cell viability,” Mol Cancer Ther 6 (1): 262-8.

Karnoub, A. E. and R. A. Weinberg (2008). “Ras oncogenes: split personalities.” Nat Rev Mol Cell Biol 9 (7): 517-31.

Luo, J., M. J, Emanuele, et al. (2009). “A genome-wide RNAi screen identifies multiple synthetic lethal interactions with the Ras oncogene.” Cell 137 (5): 835-48.

Lyapina, S., G. Cope, et al. (2001). “Promotion of NEDD-CUL1 conjugate cleavage by COP9 signalosome.” Science 292 (5520): 1382-5.

Nesbit, C. E., J. M. Tersak, et al. (1999), “MYC oncogenes and human neoplastic disease.” Oncogene 18 (19): 3004-16.

Pan, Z. Q., A, Kentsis, et al. (2004). “Nedd8 on cullin: building an expressway to protein destruction.” Oncogene 23 (11): 1985-97.

Patil, M. A., L. Gutgemann, et al. (2005), “Array-based comparative genomic hybridization reveals recurrent chromosomal aberrations and Jab1 as a potential target for 8q gain in hepatocellular carcinoma.” Carcinogenesis 26 (12): 2050-7.

Rabut, G. and M. Peter (2008). “Function and regulation of protein neddylation. ‘Protein modifications: beyond the usual suspects’ review series.” EMBO Rep 9 (10); 969-76.

Sato, Y., A. Yoshikawa, et al. (2008). “Structural basis for specific cleavage of Lys 63-linked polyubiquitin chains.” Nature 455 (7211): 358-62.

Schwechheimer, C., G. Serino, et al. (2001). “Interactions of the COP9 signalosome with the E3 ubiquitin ligase SCFT1R1 in mediating auxin response.” Science 292 (5520): 1379-82.

Shackleford, T. J. and F. X. Claret (2010). “JAB1/CSN5: a new player in cell cycle control and cancer.” Cell Div 5 (1): 26.

Verma, R., L. Aravind, et al. (2002). “Role of Rpn11 metalloprotease in deubiquitination and degradation by the 26S proteasome.” Science 291 (5593): 611-5.

Wei, N. and X. W. Deng (2003). “The COP9 signalosome.” Annu Rev Cell Dev Biol 19: 261-86.

Yao, T. and R. E. Cohen (2002). “A cryptic protease couples deubiquitination and degradation by the proteasome.” Nature 419 (6905): 403-7.

While preferred embodiment of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

1. A compound suitable for inhibiting the activity of a JAMM metalloprotease domain comprising:
an organic molecule or the pharmaceutically acceptable salt, hydrate, polymorph, solvate thereof or any combination thereof or a mixture thereof with a pharmaceutically acceptable solvent, the organic molecule having a pharmacophore moiety comprised of the fragment X—C—C—Y wherein:
the X group is nitrogen;
the Y group is oxygen or sulfur;
the carbon atoms of the pharmacophore moiety are saturated or unsaturated or a mixture thereof;
the organic molecule has an aromatic, aliphatic or aliaromatic ring framework with the X group being part of the ring framework and the Y group or the C—Y moiety being a substituent appended to the ring framework, or the organic molecule has an aromatic, aliphatic or aliaromatic framework with both of X and Y being substituents appended to the ring framework;
the ring framework is a single aliphatic or aromatic 5 or 6 member ring or a 5:5, 5:6, 6:5 or a 6:6 member aliphatic, aromatic or aliaromatic bicyclic ring, the count of members including the carbons and heteroatoms in the ring; and
the ring framework is optionally appended by one or more chemical substituents and optionally includes one or more heteroatoms in the ring framework;
provided that:
when X as nitrogen is in the ring framework, it is either a secondary or tertiary nitrogen;
when X as nitrogen is a substituent appended to the aromatic, aliphatic or aliaromatic ring framework, it is a primary, secondary or tertiary nitrogen;
provided that,
the organic molecule is not aminothiophene, hydroxylthiophene, thiolthiophene, thiol or hydroxyl pyridine, thiolpyrrole, hydroxylpyrrole, 2-aminomethylthiophene, 2-hydroxymethyllthiophene, 2-thio or hydroxymethyl-pyridine, thio or hydroxyl or aminomethyl-pyrimidine, thio or hydroxyl or aminomethyl-triazine, 2-thio or hydroxymethyl-pyrrole, thio or hydroxyl or aminomethyl-imidazole, thio or hydroxyl or aminomethyl-oxazole, thio or hydroxyl or aminomethyl-thiazole, thio or hydroxyl or aminomethyl-isoxazole, thio or hydroxyl or aminomethyl-isothiazole, a C₁ to C₆ alkyl or alkoxy substituted aforementioned derivatives, halo substituted substituted aforementioned derivatives, a phenyl or phenoxy substituted substituted aforementioned derivatives, an hydroxyl or thiol substituted substituted aforementioned derivatives, a C₁ to C₃ carboxylic acid or carboxyl ester substituted aforementioned derivatives, 8-quinolinethiol, C₁ to C₆ alkyl or alkoxy substituted 8-quinolinethiol, a halo substituted 8-quinolinethiol, a phenyl or phenoxy substituted 8-quinolinethiol, an amino substituted 8-quinolinethiol, an hydroxyl or thiol substituted 8-quinolinethiol, a C₁ to C₃ carboxylic acid or carboxyl ester substituted 8-quinolinethiol, or the disulfide dimers thereof.

2. A compound according to claim 1 wherein the aromatic, aliphatic or aliaromatic ring framework is substituted by one or more chemical substituents selected from the group consisting of halogen, CN, optionally substituted carboxyl, optionally substituted ester, optionally substituted amine, optionally substituted amide, optionally substituted thioamide, optionally substituted aliphatic or aryl carboxyl, optionally substituted aliphatic or aryl carboxyamide, optionally substituted aminoalkylamine, carboxyl, ester, alkyl aliphatic ester, amine, optionally substituted aliphatic diamine, optionally substituted aminoalkyl carboxyl, optionally substituted amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs, optionally substituted alkyl, optionally substituted alkenyl, optionally substituted alkynyl, optionally substituted alkoxy, optionally substituted alkthioxy, optionally substituted alkoxalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkylheteroaryl, optionally substituted alkheterocyclyl, optionally substituted carbocyclyl, optionally substituted alkylcarbocyclyl, carboxyl, ester, alkylcarboxyl, alkyl alkenyl ester, amine, alkylenyl diamine, aminoalkyl carboxyl, amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs and any combination thereof.

3. A compound according to claim 2 wherein the organic molecule is an 8-quinolinethiol derivative or dimer thereof and the derivative comprises the 8-quinolinethiol framework with one or more appended chemical substituents.

4. A compound suitable for inhibiting the activity of a JAMM metalloprotease domain comprising:
organic molecule or the pharmaceutically acceptable salt, hydrate, polymorph, solvate thereof or any combination thereof or a mixture thereof with a pharmaceutically acceptable solvent, the organic molecule having a zinc chelating pharmacophore moiety comprised of the fragment S—C—C—N wherein the organic molecule is 8-quinolinethiol, or a derivative thereof, or a dimer of 8-quinolinethiol or a derivative of 8-quinolinethiol dimer; wherein,
the derivative of the 8-quinolinethiol or dimer framework comprises the framework with one or more appended chemical substituents selected from the group consisting of halogen, CN, optionally substituted carboxyl, optionally substituted ester, optionally substituted amine, optionally substituted amide, optionally substituted thioamide, optionally substituted aliphatic or aryl carboxyl, optionally substituted aliphatic or aryl carboxyamide, optionally substituted aminoalkylamine, carboxyl, ester, alkyl aliphatic ester, amine, optionally substituted aliphatic diamine, optionally substituted aminoalkyl carboxyl, optionally substituted amine alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs, optionally substituted alkyl, optionally substituted alkenyl, optionally substituted alkynyl, optionally substituted alkoxy, optionally substituted alkthioxy, optionally substituted alkoxalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkyheteroaryl, optionally substituted alkheterocyclyl, optionally substituted carbocyclyl, optionally substituted alkylcarbocyclyl, carboxyl, ester, alkylcarboxyl, alkyl alkenyl ester, amine, alkylenyl diamine, aminoalkyl carboxyl, amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs and any combination thereof; and,
the 8-quinolinethiol, the derivative thereof or the dimer of 8-quinolinethiol or of the derivative of 8-quinolinethiol dimer further comprises one or more peptide substituents at the 2, 3, 4, 5, 6 and/or 7 positions of the 8-quinolinethiol framework, the peptide substituent comprising an optionally substituted peptidyl group containing from 1 to 6 natural and/or non-natural amino acid moieties, the peptidyl group being bonded to the 8-quinolinethiol framework through a linker, the linker being an alkyl amide group or an alkyl ester group wherein the amide or ester moiety forms the linking bond to the peptide substituent and the alkyl moiety is directly bonded to the 8-quinolinethiol framework or is bonded to the 8-quinolinethiol framework through an amide group, an ester group, an ether group or an amine group.

5. A compound suitable for inhibiting the activity of a JAMM metalloprotease domain comprising:
an organic molecule or the pharmaceutically acceptable salt, hydrate, polymorph, solvate thereof or any combination thereof or a mixture thereof with a pharmaceutically acceptable solvent, the organic molecule being a derivative of a 3-hydoxyl 4H-pyran-4-thione or 3-hydroxypyridine-4(1H)-thione derivative wherein the derivative comprises the 3-hydroxyl-4H-pyran-4-thione or 3-hydroxylpyridine-4(1H)-thione framework with one or more appended chemical substituents selected from the group consisting of halogen, CN, optionally substituted carboxyl, optionally substituted ester, optionally substituted amine, optionally substituted amide, optionally substituted thioamide, optionally substituted aliphatic or aryl carboxyl, optionally substituted aliphatic or aryl carboxyamide, optionally substituted aminoalkylamine, carboxyl, ester, alkyl aliphatic ester, amine, optionally substituted aliphatic diamine, optionally substituted aminoalkyl carboxyl, optionally substituted amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs, optionally substituted alkyl optionally substituted alkenyl, optionally substituted alkynyl, optionally substituted alkoxy, optionally substituted alkthioxy, optionally substituted alkoxalkyl, optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkyheteroaryl, optionally substituted alkheterocyclyl, optionally substituted carbocyclyl, optionally substituted alkylcarbocyclyl, carboxyl, ester, alkylcarboxyl, alkyl alkenyl ester, amine, alkylenyl diamine, aminoalkyl carboxyl, amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs and any combination thereof.

6. A compound suitable for inhibiting the activity of a JAMM metalloprotease domain comprising:
an organic molecule or the pharmaceutically acceptable salt, hydrate, polymorph, solvate thereof or any combination thereof or a mixture thereof with a pharmaceutically acceptable solvent, the organic molecule having the formula of a 3-hydroxyl-4H-pyran-4-thione or 3-hydroxypyridine-4(1H)-thione or a derivative thereof and the derivative comprises the 3-hydroxyl-4H-pyran-4-thione or 3-hydroxypyridine-4(1H)-thione framework with one or more appended chemical substituents selected from the group consisting of halogen, CN, optionally substituted carboxyl, optionally substituted ester, optionally substituted amine, optionally substituted amide, optionally substituted thioamide, optionally substituted aliphatic or aryl carboxyl, optionally substituted aliphatic or aryl carboxyamide, optionally substituted aminoalkylamine, carboxyl, ester, alkyl aliphatic ester, amine, optionally substituted aliphatic diamine, optionally substituted aminoalkyl carboxyl, optionally substituted amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs, optionally substituted alkyl, optionally substituted alkenyl, optionally substituted alkynyl, optionally substituted alkoxy, optionally substituted alkthioxy, optionally substituted alkoxalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkyheteroaryl, optionally substituted alkheterocyclyl, optionally substituted carbocyclyl, optionally substituted alkylcarbocyclyl, carboxyl, ester, alkylcarboxyl, alkyl alkenyl ester, amine, alkylenyl diamine, aminoalkyl carboxyl, amino alkyl ester, amino alkyl amide, halogen, alkyl halogen, alkylheterocycle, alkylaryl, alkylheteroaryl, aminoalkylaryl, aminoalkylheteroaryl, carboxylalkyaryl, carboxylalkylheteroaryl, carboxylalkylcycloalkyl, arylalkoxy, heteroarylalkoxy, cycloalkylalkoxy, the corresponding thio analogs and any combination thereof; and,
the 3-hydroxyl-4H-pyran-4-thione or 3-hydroxypyridine-4(1H)-thione or derivative thereof further comprises one or more peptide substituents at the 2, 4, 5 and/or 6 positions of the 3-hydroxyl-4H-pyran-4-thione or 3-hydroxypyridine-4(1H)-thione framework, the peptide substituent comprising an optionally substituted peptidyl group containing from 1 to 6 natural and/or non-natural amino acid moieties, the peptidyl group being bonded to the 3-hydroxyl-4H-pyran-4-thione or 3-hydroxypyridine-4(1H)-thione framework through a linker, the linker being an alkyl amide group or an alkyl ester group wherein the amide or ester moiety forms the linking bond to the peptide substituent and the alkyl moiety is directly bonded to the 3-hydroxyl-4-pyranthione framework or is bonded to the 3-hydroxyl-4-pyridinethione framework through an amide group, an ester group, an ether group or an amine group, the peptide substituent or substituents being capable of interacting with the JAMM metalloprotease domain or a biological complex containing the JAMM metalloprotease domain.

7. A compound suitable for inhibiting the activity of a JAMM metalloprotease domain, comprising: a catechol ketone of the formula:
(RO)₂C₆H₃—CO—CZ═CHY or (RO)₂C₆H₃—CO—CHC(═O)Y
or the pharmaceutically acceptable salt, hydrate, polymorpyh, solvate thereof or any combination thereof a mixture thereof with a pharmaceutically acceptable solvent;
wherein R is H or C₁ to C₆ alkyl, Z is cyano, nitro, halo or trifluoromethyl and Y is phenyl, substituted phenyl, aminophenyl, 1-indolyl, catecholyl, pyridyl, naphthyl or quinolinyl.

8. The compound according to claim 7 further comprising one or more peptide substituents on the Y group or at the 2, 5 and/or 6 positions of the catechol group, the peptide substituent comprising an optionally substituted peptidyl group containing from 1 to 6 natural and/or non-natural amino acid moieties, the peptidyl group being bonded to the Y group or the catechol group through a linker, the linker being an alkyl amide group or an alkyl ester group wherein the amide or ester moiety forms the linking bond to the peptide substituent and the alkyl moiety is directly bonded to the Y group or the catechol group or is bonded to the Y group or the catechol group through an amide group, an ester group, an ether group or an amine group, the peptide substituent or substituents being capable of interacting with the JAMM metalloprotease domain or a biological complex containing the JAMM metalloprotease domain.

9. A compound according to claim 1, 2, 3, 4, 5, 6, 7 or 8 wherein: the organic molecule in combination with a zinc cation forms a metal chelate with a K_{eq} less than 1 millimolar, as shown by a UV-visible absorption analysis of a solution of the organic molecule alone and the organic molecule complexed with zinc cation in buffered aqueous medium, the UV-visible absorption analysis being conducted to determine absorption maxima for the organic molecule alone as λᵢ and the organic molecule in a saturated chelate with zinc cation as and the equilibrium constant K_{eq} being determine by monitoring the absorption at λᵢ and λ_{f} while titrating zinc cation into an aqueous solution of the organic molecule and calculating the equilibrium constant according to the equation K_{eq} equals the concentration of the organic molecule-Zn chelate divided by the multiple of the concentrations of the organic molecule alone and the free Zn cation.

10. A compound according to claim 1, 2, 3, 4, 5, 6, 7, 8 or 9 wherein the organic molecule exhibits at least about a 50% inhibition of metalloprotease activity of a JAMM metalloprotease domain containing protein alone or as part of a signalosome complex or part of a 26S proteasome complex, which inhibition is determined by conducting a biochemical assay of the ability of the compound to inhibit the ability of the JAMM metalloprotease domain to cleave a monoubiquitin, a multiubiquitin chain or a ubiquitin-like modifier from a protein substrate or ubiquitin from a K63-linked ubiquitin chain, the concentration of the organic molecule being no more man about 500 micromolar.

11. A compound according to claim 1, 2, 3, 4, 5, 6, 7 or 8 wherein the peptide substituent and the optional chemical substituent or substituents do not cause a significant decrease in the inhibitory activity relative to the inhibitory activity of a corresponding standard compound, 8-quinolinethiol or 3-hydroxyl-4H-pyran-4-thione or 3-hydroxypyridine-4(1H)-thione, in the JAMM domain inhibition assay.

12. A compound according to one of claims 4, 6 or 8 wherein the peptide substituent is an epitopal substrate for the JAMM metalloprotease domain or is a single chain hypervariable region chain from a humanized or chimeric monoclonal antibody to the protein containing the JAMM metalloprotease domain or the signalsome complex containing protein.

13. A pharmaceutical formulation comprising a pharmaceutically acceptable carrier and an amount of a compound of any one of claims 1 through 8 effective to inhibit the metalloprotease activity of a JAMM metalloprotease containing protein.

14. A method for screening for a compound that inhibits the metalloprotease activity of a protein containing a JAMM metalloprotease domain, comprising:
selecting a candidate from a group of organic molecules;
testing the selected candidate in a JAMM domain inhibition assay comprising
combining
a JAMM enzymatic material selected from the group consisting of a JAMM domain containing protein, a signalosome complex and a 26S proteasome complex containing the JAMM protein,
and a protein substrate selected from the group consisting of a protein modified by a ubiquitin, a protein modified by a ubiquitin-like modifier and a protein modified by a ubiquitin chain to produce an enzymatic medium
wherein the protein substrate is modified with a tag that is detectable by measurement of molecular weight, spectroscopic interaction or chromatographic R_{f} determination,
conducting a first measurement of the enzymatic medium relative to the protein substrate alone wherein the first measurement is made by a detection of the tag,
combining the selected candidate with the protein substrate and adding the JAMM enzymatic material to produce a candidate medium,
conducting a second measurement of the candidate medium relative to the protein substrate alone wherein the second measurement is made by detection of the tag,
comparing the first and second measurements to identify a candidate that demonstrates at least about a 50% inhibition at a concentration of no more than 500 micromolar in the candidate medium, the difference between the first and second measurements being at least about 50% with the second measurement being greater than the first measurement; WHEREIN,
the organic molecule is selected from the group consisting of subgroups a, b, c, d, e and f, these subgroups being:
a) an organic molecule having a pharmacophore moiety comprised of the fragment X—C—C—Y wherein:
X is nitrogen;
Y is oxygen or sulfur;
the carbon atoms of the pharmacophore moiety are saturated or unsaturated or a mixture thereof;
the organic molecule has an aromatic, aliphatic or aliaromatic ring framework with the X group being part of the ring framework and the Y group or the C—Y moiety being a substituent appended to the ring framework, or the organic molecule has an aromatic, aliphatic or aliaromatic ring framework with both or X and Y being substituents appended to the ring framework;
the framework is a single aromatic or aliphatic 5 or 6 member ring or a 5:5, 5:6, 6:5 or a 6:6 member aromatic, aliphatic or aliaromatic bicyclic ring, the count of members including the carbons and heteroatoms in the ring; and
the ring framework is optionally appended by one or more chemical substituents and optionally includes one or more heteroatoms in the ring framework;
provided that:
when X as nitrogen is in the ring framework, it is either a secondary or tertiary nitrogen;
when X as nitrogen is a substituent appended to the ring framework, it is a primary, secondary or tertiary nitrogen;
b) the organic molecule is 8-quinolinethiol, or a derivative thereof, or a dimer of 8-quinolinethiol or a derivative of 8-quinolinethiol dimer; wherein,
the derivative of the 8-quinolinethiol or dimer framework comprises the framework with one or more appended chemical substituents; and,
the 8-quinolinethiol, the derivative thereof or the dimer of 8-quinolinethiol or of the derivative of 8-quinolinethiol dimer further comprises one or more peptide substituents at the 2, 3, 4, 5, 6 and/or 7 positions of the 8-quinolinethiol framework, the peptide substituent comprising an optionally substituted peptidyl group containing from 1 to 6 natural and/or non-natural amino acid moieties, the peptidyl group being bonded to the 8-quinolinethiol framework through a linker, the linker being an alkyl amide group or an alkyl ester group wherein the amide or ester moiety forms the linking bond to the peptide substituent and the alkyl moiety is directly bonded to the 8-quinolinethiol framework or is bonded to the 8-quinolinethiol framework through an amide group, an ester group, an ether group or an amine group;
c) an organic molecule is a derivative of 3-hydoxyl 4-pyrothione or 3-hydroxypyridine-4(H)-thione and the derivative comprises the 3-hydroxyl-4-pyrothione or 3-hydroxypyridine-4(H)-thione framework with one or more appended chemical substituents;
d) an organic molecule is 5-hydoxyl 4-pyrothione, 3-hydroxypyridine-4(H)-thione or a derivative thereof and the derivative comprises the 3-hydroxyl-4-pyrothione or 3-hydroxypyridine-4(H)-thione framework with one or more appended chemical substituents; and,
the 3-hydoxyl 4-pyrothione, 3-hydroxypyridine-4(H)-thione or derivative thereof further comprises one or more peptide substituents at the 2, 4, 5 and/or 6 positions of the 3-hydroxyl-4-pyrothione or pyrothione or 3-hydroxypyridine-4(H)-thione framework, the peptide substituent comprising an optionally substituted natural or non-natural peptidyl group containing from 2 to 6 amino acid moieties, the peptidyl group being bonded to the 3-hydroxyl-4-pyrothione or 3-hydroxypyridine-4(H)-thione framework through a linker, the linker being an alkyl amide group or an alkyl ester group wherein the amide or ester moiety forms the linking bond to the peptide substituent and the alkyl moiety is directly bonded to the 3-hydroxyl-4-pyrothione framework or is bonded to the 3-hydroxyl-4-pyrothione or 3-hydroxypyridine-4(H)-thione framework through an amide group, an ester group, an ether group or an amine group, the peptide substituent or substituents being capable of interacting with the JAMM metalloprotease domain or a biological complex containing the JAMM metalloprotease domain;
e) an organic molecule from a group of catechol ketones of the formula:
(RO)₂C₆H₃—CO—CZ═CHY or (RO)₂C₆H₃—CO—CHZC(═O)Y
wherein R is hydrogen or alkyl, Z is cyano, nitro, halo or trifluoromethyl and Y is phenyl, substituted phenyl, aminophenyl, 1-indolyl, catecholyl, pyridyl, naphthyl or quinolinyl, and
f) an organic molecule of group e) with one or more peptide substituents on the Y group or at the 2, 5 and/or 6 positions of the catechol group, the peptide substituent comprising an optionally substituted natural or non-natural peptidyl group containing from 2 to 6 amino acid moieties, the peptidyl group being bonded to the Y group or the catechol group through a linker, the linker being an alkyl amide group or an alkyl ester group wherein the amide or ester moiety forms the linking bond to the peptide substituent and the alkyl moiety is directly bonded to the Y group or the catechol group or is bonded to the Y group or the catechol group through an amide group, an ester group, an ether group or an amine group.

15. A method according to claim 14 wherein the chemical substituent or substituents of subgroups a, b, c, d, e and f are selected from the group consisting of halogen, CN, optionally substituted carboxyl, optionally substituted ester, optionally substituted amine, optionally substituted amide, optionally substituted thioamide, optionally substituted aliphatic or aryl carboxyl, optionally substituted aliphatic or aryl carboxyamide, optionally substituted aminoalkylamine, carboxyl, ester, alkyl aliphatic ester, amine, optionally substituted aliphatic diamine, optionally substituted aminoalkyl carboxyl, optionally substituted amino alkyl ester, amino alkyl amide, halogen, alkyl halogen, alkylheterocycle, alkylaryl, alkylheteroaryl, aminoalkylaryl, aminoalkylheteroaryl, carboxylalkylaryl, carboxylalkylheteroaryl, carboxylalkylcycloalkyl, arylalkoxy, heteroarylalkoxy, cycloalkylalkoxy, the corresponding thio analogs, optionally substituted alkyl, optionally substituted alkenyl, optionally substituted alkynyl, optionally substituted alkoxy, optionally substituted alkthioxy, optionally substituted alkoxalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkylheteroaryl, optionally substituted alkheterocyclyl, optionally substituted carbocyclyl, optionally substituted alkylcarbocyclyl, carboxyl, ester, alkylcarboxyl, alkyl alkenyl ester, amine, alkylenyl diamine, aminoalkyl carboxyl, amino alkyl ester, amino alkyl amide, halogen, alkyl halogen, alkylheterocycle, alkylaryl, alkylheteroaryl, aminoalkylaryl, aminoalkylheteroaryl, carboxylalkyaryl, carboxylalkylheteroaryl, carboxylalkylcycloalkyl, arylalkoxy, heteroarylalkoxy, cycloalkylalkoxy, the corresponding thio analogs and any combination thereof.

16. A method for treatment of a human disorder characterized by abnormal regulatory peptide degradation resulting in excessive cell proliferation or cell signaling, comprising administering an effective amount of a compound of any of claims 1-8 and 10 so that the abnormal regulatory peptide degradation is ameliorated, reduced or inhibited.

17. A method for treatment of a human disorder characterized by abnormal regulatory peptide degradation resulting in excessive cell proliferation or cell signaling, comprising administering a pharmaceutical formulation of claim 16 so that the abnormal regulatory peptide degradation is ameliorated, reduced or inhibited.

18. A method according to claim 16 or 17 wherein the human disorder is a cancer or immune disorder.

19. A method according to claim 6 or 17 wherein the human disorder is a cancer resulting from overexpression of c-Myc or expression of an oncogenic form of the K-Ras protein.

20. A method for the inhibition or amelioration of JAMM metalloprotease domain activity in a human patient suffering from abnormal JAMM metalloprotease domain activity on ubiquitin modified proteins, comprising administering to the patient an effective amount of a compound of any of claims 1-8, or a pharmaceutical composition of claim 10 so that the abnormal JAMM metalloprotease domain activity is ameliorated, reduced or inhibited.

21. An assay for the determination of inhibition of JAMM metalloprotease domain activity by a potential inhibitory candidate comprising:
combining
a JAMM enzymatic material selected from the group consisting of a JAMM domain containing protein, a signalosome complex and a 26S proteasome complex containing the JAMM protein,
and a protein substrate selected from the group consisting of a protein modified by a ubiquitin, a protein modified by a ubiquitin-like modifier and a protein modified by a ubiquitin chain to produce an enzymatic medium
wherein the protein substrate is modified with a tag that is detectable by measurement of molecular weight, spectroscopic interaction or chromatographic R_{f} determination,
conducting a first measurement of the enzymatic medium relative to the protein substrate alone wherein the first measurement is made by a detection of the tag,
combining the potential inhibitory candidate with the protein substrate and adding the JAMM enzymatic material to produce a candidate medium,
conducting a second measurement of the candidate medium relative to the protein substrate alone wherein the second measurement is made by detection of the tag,
comparing the first and second measurements to identify a candidate that demonstrates at least about a 50% inhibition at a concentration of no more than 500 micromolar in the candidate medium, the difference between the first and second measurements being at least about 50% with the second measurement being greater than the first measurement.

22. A compound according to any of claims 1-8 wherein the one or more appended chemical substituents are selected from the group consisting of halogen, optionally substituted alkyl, optionally substituted alkoxy, optionally substituted alkthioxy, optionally substituted amino, optionally substituted, carboxyl, optionally substituted aryl, optionally substituted heteroaryl, optionally substituted carbocyclyl, optionally substituted heterocyclyl and any combination thereof; or in the alternative,
are selected from the group consisting of halogen, optionally substituted alkyl, optionally substituted amino, optionally substituted carboxyl, optionally substituted aryl, optionally substituted heteroaryl, optionally substituted carbocyclyl, optionally substituted heterocyclyl and any combination thereof; or in the alternative,
are selected from the group consisting of optionally substituted alkyl, optionally substituted aryl, optionally substituted heteroaryl, optionally substituted carbocyclyl, optionally substituted heterocyclyl and any combination thereof; or in the alternative,
are selected from the group consisting of optionally substituted alkyl, optionally substituted aryl, optionally substituted heteroaryl and any combination thereof, or in the alternative,
are selected from the group consisting of halogen, optionally substituted alkoxy, optionally substituted amino, optionally substituted carboxyl and any combination thereof; or in the alternative,
are selected form the group consisting of optionally substituted aryl, optionally substituted, heteroaryl and any combination thereof.

23. A compound according to claim 22 wherein the one or more appended chemical substituents numbers from one to four; or in the alternative, numbers from one to two.

24. A compound according to one of claim 22 wherein the peptide substituents number one or two.

25. A method for screening according to claim 15 wherein the one or more appended chemical substituents are selected born the group consisting of halogen, optionally substituted alkyl, optionally substituted alkoxy, optionally substituted alkthioxy, optionally substituted amino, optionally substituted carboxyl, optionally substituted aryl, optionally substituted heteroaryl, optionally substituted carbocyclyl, optionally substituted heterocyclyl and any combination thereof, or alternatively,
wherein the one or more appended chemical substituents are selected from the group consisting of halogen, optionally substituted alkyl, optionally substituted amino, optionally substituted carboxyl, optionally substituted aryl, optionally substituted heteroaryl, optionally substituted carbocyclyl, optionally substituted heterocyclyl and any combination thereof, or alternatively,
wherein the one or more appended chemical substituents are selected from the group consisting of optionally substituted alkyl, optionally substituted aryl, optionally substituted heteroaryl, optionally substituted carbocyclyl, optionally substituted heterocyclyl and any combination thereof, or alternatively,
wherein the one or more appended chemical substituents are selected from the group consisting of optionally substituted alkyl, optionally substituted aryl, optionally substituted heteroaryl and any combination thereof, or alternatively,
wherein the one or more appended chemical substituents are selected from the group consisting of halogen, optionally substituted alkoxy, optionally substituted amino, optionally substituted carboxyl and any combination thereof, or alternatively,
wherein the one or more appended chemical substituents are selected form the group consisting of optionally substituted aryl, optionally substituted heteroaryl and any combination thereof, or alternatively,
wherein the one or more appended chemical substituents numbers from one to four, or alternatively,
wherein the one or more appended chemical substituents numbers from one to two; and
each of the alternatives may be combined in any combination with any other alternative.

26. A compound according to claim 1 wherein the chemical substituents appended, to the ring framework are selected from the group consisting of halogen, CN, optionally substituted alkyl, optionally optionally substituted alkenyl, optionally substituted alkynyl, optionally substituted alkoxy, optionally substituted alkthioxy, optionally substituted alkoxalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkyheteroaryl, optionally substituted alkheterocyclyl, optionally substituted, carbocyclyl, optionally substituted alkylcarbocyclyl, and any combination thereof.

27. A compound according to claim 1, which comprises Formula (V): 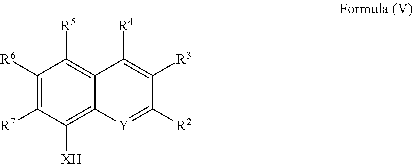
or a pharmaceutically acceptable salt thereof, wherein:
X is sulfur and Y is independently selected from the group consisting of O, N, and S;
R², R³, R⁴, R⁵, R⁶ and R⁷ are each independently selected from hydrogen, halogen, CN, optionally substituted alkyl, optionally substituted alkenyl, optionally substituted alkynyl, optionally substituted alkoxy, optionally substituted alkthioxy; optionally substituted alkoxalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkyheteroaryl, optionally substituted alkheterocyclyl carbocyclyl, optionally substituted alkylcarbocycl; optionally substituted natural or non-natural peptidyl containing 2˜6 amino acids moiety, optionally substituted natural or non-natural peptidyl through a linker and the linker is selected from optionally substituted alkyl, optionally substituted alkoxy, optionally substituted amine, optionally substituted carbonyl and optionally substituted, carboxylic acid.

28. A compound according to claim 1 which comprises Formula (VI): 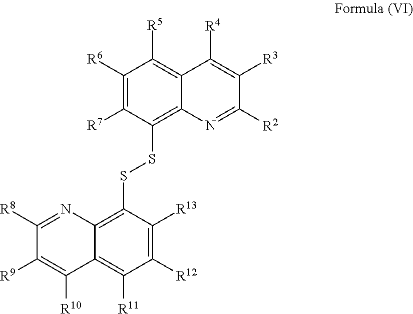
or a pharmaceutically acceptable salt thereof, wherein:
R2, R3, R4, R5, R6, R7, R8, R9, R10, R11, R12 and R13 are each independently selected from hydrogen, halogen, CN, optionally substituted alkyl, optionally substituted alkenyl, optionally substituted alkynyl, optionally substituted alkoxy, optionally substituted alkthioxy, optionally substituted alkoxalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkyheteroaryl, optionally substituted alkheterocyclyl, carbocyclyl, optionally substituted alkylcarbocycl; optionally substituted natural or non-natural peptidyl containing 2˜6 amino acids moiety, optionally substituted natural or non-natural peptidyl through a linker and the linker is selected from optionally substituted alkyl, optionally substituted alkoxy, optionally substituted amine, optionally substituted carbonyl and optionally substituted carboxylic acid.

29. A compound according to claim 1 which comprises Formula (VII): 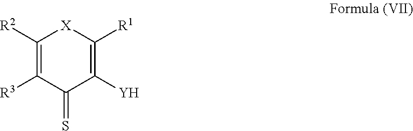
or a pharmaceutically acceptable salt thereof, wherein:
X, Y are independently selected from the group consisting of O, N, S;
R1, R2 and R3 are each independently selected from hydrogen, halogen, CN, optionally substituted alkyl, optionally substituted alkenyl, optionally substituted alkynyl, optionally substituted alkoxy, optionally substituted alkthioxy, optionally substituted alkoxalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkyheteroaryl, optionally substituted alkheterocyclyl, carbocyclyl, optionally substituted alkylcarbocycl; optionally substituted natural or non-natural peptidyl containing 2˜6 amino acids moiety, optionally substituted natural or non-natural peptidyl through a linker and the linker is selected from optionally substituted alkyl, optionally substituted alkoxy, optionally substituted amine, optionally substituted carbonyl and optionally substituted carboxylic acid.

30. A compound according to claim 14 which comprises Formula (VIII): 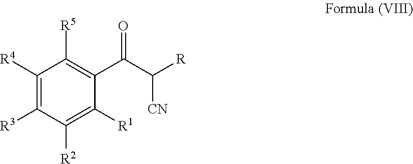
or a pharmaceutically acceptable salt thereof, wherein:
R, R1, R2, R3, R4 and R5 are each independently selected from hydrogen, halogen, CN, optionally substituted alkyl, optionally substituted alkenyl, optionally substituted alkynyl, optionally substituted alkoxy, optionally substituted alkthioxy, optionally substituted alkoxalkyl; optionally substituted alkoxaryl; optionally substituted amino, optionally substituted carbonyl, optionally substituted carboxylic acid, optionally substituted alkoxheteroaryl, optionally substituted alkoxheterocycyl, optionally substituted aryl, optionally substituted heteroaryl, optionally substituted heterocyclyl, optionally substituted alkaryl, optionally substituted alkyheteroaryl, optionally substituted alkheterocyclyl carbocyclyl, optionally substituted alkylcarbocycl; optionally substituted natural or non-natural peptidyl containing 2˜6 amino acids moiety, optionally substituted natural or non-natural peptidyl through a linker and the linker is selected from optionally substituted alkyl, optionally substituted alkoxy, optionally substituted amine, optionally substituted carbonyl and optionally substituted carboxylic acid.

31. A compound according to any of claims 1 through 8 comprising the organic molecule or the pharmaceutically acceptable salt thereof.

32. A compound according to any of claims 1 through 8 wherein the organic molecule has a framework selected from the following group and one or two chemical substituents positioned at the designated positions:
a) 8-thioquinoline framework, positions 2, 3, 4, 5, 6, 7,
b) 2-mercaptomethylpyridine framework, positions 2, 3, 4, 5,
c) 5,6,7,8-tetrahydro-8-thioquinoline framework, positions 2, 3, 4, 5, 6, 7,
d) 1-mercapto-4-aza-2,3-dihydroindane framework, positions 2, 3, 5, 6, 7,
e) 4-mercaptoindole framework, positions 2, 3, 5, 6, 7,
f) 4-mercaptobenzoimidazole framework, positions 2, 5, 6, 7,
g) 4-mercaptobenzoxazole framework, positions 2, 5, 6, 7,
h) 4-mercaptobenzthiazole framework, positions 2, 5, 6, 7,
i) 8-mercaptoquinazoline framework, positions 2, 6, 7, 8,
j) 3-hydroxy-4H-pyran-4-thione framework, positions 2, 5, 6,
k) 8-mercapto-4H-quinazolin-4-one framework, positions 2, 6, 7, 8,
l) 8-mercapto-2H-4H-quinazolin-2,4-dione framework, positions 6, 7, 8,
m) 3-mercaptopyridinothiophene framework, positions 2, 3, 5, 6, 7,
n) a 5 membered single ring framework with X—C—C—Y of aromatic character, or of aliphatic character, or of aliaromatic character, or any combination thereof as defined in the summary of the invention, positions 2, 3, 4, 5
o) a 6 membered single ring framework with X—C—C—Y of aromatic character, or of aliphatic character, or of aliaromatic character, or any combination thereof as defined in the summary of the invention, positions 2, 3, 4, 5, 6,
p) a 5:5 membered bicyclic ring framework with X—C—C—Y of aromatic character, or of aliphatic character or of aliaromatic character or any combination thereof as defined in the summary of the invention, positions 2, 3, 4, 5, 6, 7, 8,
q) a 5:6 or a 6:5 membered bicyclic ring framework with X—C—C—Y of aromatic character, or of aliphatic character or of aliaromatic character or any combination thereof as defined in the summary of the invention, positions 2, 3, 4, 5, 6, 7, 8, 9,
r) a 6:6 membered bicyclic ring framework with X—C—C—Y of aromatic character, or of aliphatic character or of aliaromatic character or any combination thereof as defined in the summary of the invention, positions 2, 3, 4, 5, 6, 7, 8, 9.

33. A compound according to claim 32 wherein the one or two chemical substituents are selected from the following group:
a) Alkyl and branched alkyl of 1 to 6 carbons,
b) Alkoxy and branched alkoxy of 1 to 6 carbons,
c) Amine,
d) Carboxylic acid,
e) Carboxylic ester wherein the alkoxy group of the ester is from 1 to 6 branched or straight carbons or the alcohol esterifying group is phenoxy,
f) Branched or straight alkylenyl carboxylic acid or ester of 2 to 7 carbons in the alkylenyl group and 1 to 6 branched or straight carbons in the ester group,
g) Branched or straight alkylenyl amine of 1 to 6 carbons,
h) Branched or straight perfluoroalkyl of 1 to 6 carbons,
i) Branched or straight trifluoroalkyl of 1 to 6 carbons wherein the trifluoro group is on the terminating or end carbon,
j) Hydroxyl,
k) Branched or straight alkylenyl hydroxyl of 1 to 6 carbons,
l) Carboxamide eg, —CONH₂
m) Aminocarbonylalkyl, eg —NHCOR wherein R is alkyl of 1 to 6 carbons,
n) Branched or straight alkylenyl carboxyamido of 1 to 6 carbons,
o) Branched or straight, alkylenylcarboxyamide of 1 to 6 carbons, e.g., —RCONH₂,
p) Alkyleneaminocarbonylalkyl, eg., —RNHCOR, wherein the alkylenyl is branched or straight and is 1 to 6 carbons and the alkyl is branched or straight and is 1 to 6 carbons,
q) N-substituted carboxamide, wherein the N substituent is an aryl group, heteroaryl group or heterocycle group as defined in the DEFINITIONS section, eg., —CONHAr or —CONHHet,
r) N-substituted carboxamide wherein the N substituent is an alkaryl group, a alkyheteroaryl group or a alkheterocycle group as defined in the DEFINITIONS section, and wherein the “alk” group is an alkylenyl or branched alkylenyl group of 1 to 6 carbons, eg., —CONN—R—Ar or —CONH—R-Het,
s) N-substituted carboxamide wherein the N substituent is a branched or straight alkyl group of 1 to 10 carbons, the polyfluorinated version thereof or a substituted version thereof, eg., —CONH—R, wherein the substituent of the alkyl group is halogen, cyano, carboxyl, ester of 1 to 6 branched or straight chain carbons in the alkoxy or phenoxy portion, carboxamide, sulfoxamide, alkoxy of 1 to 6 carbons, urea, carbamate of 1 to 10 carbons, amine, mono or dialkyl amine having from 1 to 6 carbons in the alkyl group with the alkyl group being straight or branched, hydroxyalkyl of 1 to 10 branched or straight chain carbons or a cycloalkyl group as defined in the DEFINITIONS section,
t) Preferred aryl, heteroaryl and heterocycle groups for q and r include phenyl, halogen substituted phenyl, aminophenyl, benzoic acid, tolyl, xylyl anisolyl, trifluoromethylphenyl, benzyl, tetrahydrofuran, pyrrolidinyl, tetrahydronaphthalene, cyclohexyl or alkyl substituted cyclohexyl with the alkyl group having 1 to 6 carbons, cyclohexyl or alkyl substituted cyclohexyl with the alkyl group having 1 to 6 carbons, cyclopentyl or alkyl substituted cyclopentyl with the alkyl group having 1 to 6 carbons, pyrazolyl, imidazolyl, piperidinyl, piperazinyl, pyrimidinyl, morpholinyl, pyrrolyl, thiophenyl, substituted versions of any of the foregoing aryl, heteroaryl or heterocycle groups wherein the chemical substituent is halogen, cyano, carboxyl, ester of 1 to 10 branched or straight chain carbons in the alkoxy or phenoxy portion, amine, carboxamide, sulfoxamide, urea, carbamate of 1 to 10 carbons, hydroxyl, thiol, alkoxy, anisolyl, phenyl, benzyl or a cycloalkyl group as defined in the DEFINITIONS section,
u) Derivatives of p, q and r wherein the N of the carboxamide has a second substituent and the second substituent is a branched or straight chain alkyl of 1 to 6 carbons,
v) N-substituted carboxyamide wherein the N substituent is a mono, di, tri or tetra amino acid and the amino acid moieties include glycinyl, alaninyl, leucinyl, valinyl, phenylalaninyl, lysinyl, argininyl, histidinyl, serinyl, aspariginyl, glutaminyl, aspartic, glutamic such that the amino acid moieties may be combined in any combination of two, three or four moieties including but not limited to a tetramer of four different moieties, a tetramer of two and two different moieties, a tetramer of three of one moiety and one of a different moiety, a trimer of two of one moiety and one of another moiety or a trimer of three different moieties, a dimer of two different moieties of of the same moiety, and a monomer of any of the designated moieties, wherein, the nitrogen of an amino acid moiety may serve as the nitrogen of the carboxyamide group; the C-terminus of the amino acid monomer, dimer or trimer may be a carboxylic acid or a carboxamide and the order of amino acid moieties in the tetramer trimer or dimer may be any order;
w) And any combination of the chemical substituents a-u.

34. A compound of claim 33 wherein each framework is individually combined with each individual species of chemical substituent to provide multiple species of each framework, the multiple species being indicated by each individual species of chemical substituent.

35. The compound of claim 33 wherein each individual species of framework combined with a chemical substituent is combined a second time with a different species of chemical substituent.

36. A compound according to claim 26, 33, 34 or 35 wherein the number of chemical substituents appended to the framework is 1, 2, 3 or 4, or alternatively and preferably 1 or 2, or alternatively and more preferably 1.

37. A compound according to claim 1, 2, 11, 22-24, 26, 32 or 33 wherein the aromatic, aliphatic or aliaromatic framework is a 5 membered single ring

38. A compound according to claim 1, 2, 11, 22-24, 26, 32 or 33 wherein the aromatic, aliphatic or aliaromatic framework is a 6 membered single ring.

39. A compound according to claim 1, 2, 11, 22-24, 26, 32 or 33 wherein the aromatic, aliphatic or aliaromatic framework is a 5:6 or 6:5 bicyclic ring.

40. A compound according to claim 1, 2, 11, 22-24, 26, 32 or 33 wherein the aromatic, aliphatic or aliaromatic framework is a 5:5 bicyclic ring.

41. A compound according to claim 1, 2, 11, 22-24, 26, 32 or 33 wherein the aromatic, aliphatic or aliaromatic framework is a 6:6 bicyclic ring.

42. A compound according to claim 37 through 41 wherein the framework is aliaromatic or aromatic.

43. A compound according to claim 42 wherein the framework is aromatic.

44. A compound according to claim 37 through 43 wherein Y is sulfur.
